(19) United States
(12) Patent Application Publication
(10) Pub. No.: US 2022/0288169 A1
(43) Pub. Date: Sep. 15, 2022

(54) COMPOSITIONS AND METHODS FOR THE TREATMENT AND PREVENTION OF HYPOGLYCEMIC COMPLICATIONS

(71) Applicant: The Board of Supervisors of Louisiana State University and Agriculyural and Mechanical College, Baton Rouge (US)

(72) Inventor: David McDougal, Springfield, LA (US)

(21) Appl. No.: 17/612,979

(22) PCT Filed: May 26, 2019
(86) PCT No.: PCT/US2020/034548

§ 371 Date: Nov. 19, 2021

Related U.S. Application Data
(60) Provisional application No. 62/852,775, filed on May 24, 2019.

(51) Int. Cl. A61K 38/22; A61K 38/28; A61P 3/08

(52) U.S. Cl. CPC A61K 38/2264; A61K 38/28; A61P 3/08

(57) ABSTRACT

This invention is directed to compositions and methods for the treatment and prevention of hypoglycemic complications. Specifically, aspects of the invention are drawn to the use of leptin for the treatment and prevention of hypoglycemic complications.

This application is an International Application which claims priority from U.S. provisional patent application No. 62/852,775 filed on May 24, 2019, the entire contents of which is incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. U54 GM104940 awarded by the National Institutes of Health. The government has certain rights in the invention.

### FIELD OF THE INVENTION

This invention is directed to compositions and methods for the treatment and prevention of hypoglycemic complications. Specifically, aspects of the invention are drawn to the use of leptin for the treatment and prevention of hypoglycemic complications.

### BACKGROUND OF THE INVENTION

Hypoglycemic complications are a major barrier to the successful treatment of individual's with diabetes. Repeated bouts of severe-hypoglycemia lead to the loss of the normal physiological response to hypoglycemia, called the counter-regulatory response.

The need for clinical intervention cannot be understated. There is currently no pharmaceutical intervention available to restore the loss of the counter-regulatory response. The only treatment strategy is the stringent avoidance of hypoglycemia, which is extremely difficult to accomplish clinically, such as in high-risk patients. Approximately six million diabetic patients are dependent on insulin in the US. The typical insulin-treated patient with diabetes experiences at least one severe episode of hypoglycemia each year, and approximately 30% will require emergency medical care. One third of patients that require emergency medical care will be hospitalized at an estimated cost of $21,000 per patient. An average commercial health care plan in the US (10 million members) spends $170 million annually just on hospitalization costs associated with severe hypoglycemia. The annual cost of severe hypoglycemia in diabetic patients in the US is estimated to be between $5 billion and $12 billion.

### SUMMARY OF THE INVENTION

The present invention provides a method of treating and/or preventing complications associated with hypoglycemia, the method comprising administering to a subject in need thereof a therapeutically effective amount of a composition comprising leptin, fragments thereof, and/or leptin receptor agonists.

Aspects of the invention are drawn to a method of preventing a hypoglycemia-associated complication. In embodiments, the method comprises administering to a subject in need thereof a therapeutically effective amount of a composition comprising leptin, a fragment thereof, and/or a leptin receptor agonist.

Aspects of the invention are also drawn towards a method of treating a hypoglycemia-associated complication. In embodiments, the method comprises administering to a subject in need thereof a therapeutically effective amount of a composition comprising leptin, a fragment thereof, and/or a leptin receptor agonist.

In an embodiment, the hypoglycemia-associated complication is HAAF.

In an embodiment, the composition is administered prior to the onset of severe hypoglycemia.

In an embodiment, the composition is administered at about the same time as the onset of severe hypoglycemia.

In an embodiment, the leptin comprises human recombinant leptin.

In an embodiment, the method comprises administering a short course of leptin.

In an embodiment, the method comprises chronic administration of leptin.

In an embodiment, the subject in need thereof is afflicted with diabetes.

In an embodiment, the composition further comprises insulin.

Aspects of the invention are also drawn to a therapeutic combination. In an embodiment, the composition comprises leptin, a fragment thereof, or a leptin receptor agonist, and at least one additional active agent.

In an embodiment, the composition further comprises a pharmaceutically acceptable carrier, diluent, or excipient.

In an embodiment, the at least one additional active agent comprises insulin or an insulin secretagogue.

In an embodiment, the at least one additional active agent comprises insulin.

In an embodiment, the insulin secretagogue comprises sulfonylurea or glinides.

Other objects and advantages of this invention will become readily apparent from the ensuing description.

### BRIEF DESCRIPTION OF THE FIGURES

FIG. 1 shows six days of 60% caloric restriction leads to significant reductions in body weight and blood glucose while inducing hyperphagia and sustained reductions in blood glucose following refeeding.

FIG. 2 show hypoglycemic counter-regulation is impaired following exposure to six days of 60% caloric restriction and is preceded by hypoleptinemia.

FIG. 3 shows impaired hypoglycemic counter regulation following exposure to six days of 60% caloric restriction is rescued by leptin treatment.

FIG. 4 shows temporal changes in absolute fat mass, lean mass, and body composition following refeeding after 60% caloric restriction. Six days of 60% caloric restriction in mice led to significant reductions in both fat mass and lean mass (Day 0, Panels A and B). This effect persisted during the first two days of the refeeding period. In terms of overall body composition, this reduction in fat mass represented a 50% reduction in % fat mass relative to ad libitum (Ad-lib) fed controls (C). This dramatic reduction in % fat mass led to CR mice having significantly higher % lean mass on the final day of caloric restriction relative to Ad-lib mice (Day 0, panel D). This effect reversed on the first day of the refeeding period before % lean mass equalized relative to Ad-lib mice on the second and fourth day of the refeeding period (D). Data were analyzed via two-way ANOVA. Group differences on each Day were determined via Bonferroni's multiple comparison test with significance levels as follows: ***-p<0.001. n=52 per group days 1-17, n=35 per group days 18-19, and n=22 per group on day 20.

FIG. 5 shows temporal changes in hormones and metabolites following refeeding after 60% caloric restriction. Six days of 60% caloric restriction in mice led to significant reductions in fasting glucose, leptin, and hepatic glycogen levels (Day 0, Panels A, B, and C), while significantly increasing fasting ghrelin and β-hydroxybutyrate levels (Day 0, Panels D and F). Following a single day of refeeding, levels of hepatic glycogen, ghrelin, and β-hydroxybutyrate normalized and were not significantly different from ad libitum fed control mice for the remainder of the refeeding period (Day 1, 2, and 4, Panels B, D, and F). In contrast, fasting glucose and leptin remained significantly lower during the first day of refeeding in mice exposed to 60% caloric restriction before returning to levels similar to Ad-lib mice on Days 2 and 4 of the refeeding period (Panels A and C). Fasting glucagon levels were not significantly different between the two groups on the final day of caloric restriction (Day 0, Panel E), nor were there any differences between the groups during the refeeding period (Days 1, 2, and 4, Panel E). Data were analyzed via two way ANOVA. Group differences on each Day were determined via Bonferroni's multiple comparison test with significance levels as follows: *-p<0.05; **-p<0.01; ***-p<0.001. n=7-8/group (A and C); n=5-8/group (D, E, and F); n=4-6/group (B).

FIG. 6 shows hypoglycemic counter-regulation is impaired following exposure to six days of 60% caloric restriction. One, two, and four days following refeeding, hypoglycemic counter-regulation was assessed via a 60 minute hypoglycemic insulin tolerance test (ITT) in mice exposed to a six-day 60% caloric restriction paradigm (CR, black) and ad libitum fed controls (Ad-lib, grey). In order to overcome differences in baseline blood glucose levels between groups, a variable dose of insulin (IP) was used to produce equivalent exposure to hypoglycemia (A). Following a single day of refeeding, mice exposed to 60% caloric restriction displayed a significant reduction in hypoglycemia-stimulated glucagon release (Day 1, Panel B). There was no significant difference in hypoglycemia-stimulated glucagon release between caloric restriction and Ad-lib mice two and four days following refeeding (Day 2 and 4, Panel B). Data were analyzed via two-way ANOVA. Group differences were determined via Bonferroni's multiple comparison test with significance levels as follows: *-p<0.05; **-p<0.01; n=7-8/group.

FIG. 7 shows exposure to severe caloric restriction leads to deficits in hypoglycemia counter-regulation, which can be rescued by leptin supplementation.

FIG. 8 shows overview of Experiment 1 with groups.

FIG. 9 shows overview of clinical study design, measurement, aims and timing of study visits.

FIG. 10 shows schedule of assessments.

FIG. 11 shows ix days of 60% caloric restriction leads to significant reductions in body weight and blood glucose while inducing hyperphagia and sustained reductions in blood glucose following refeeding. Following a five day acclimation period and six days of baseline food intake data, wild type B6 male mice were assigned to either a 60% caloric restriction paradigm (CR mice; black circles) or ad libitum access to chow (Ad-lib mice; grey squares) for six days. Following the caloric restriction period, both groups of mice, CR and Ad-lib, were given ad libitum access to chow for four days (see top border for timing of each experimental period). CR mice experienced significant reductions in both body weight and blood glucose during the CR paradigm with both measurements reaching their nadir on the final day of the paradigm. The reduction in body weight was reversed within three days of refeeding, while blood glucose levels remained lower than those of Ad-lib mice following refeeding. CR mice also displayed a marked hyperphagia during the refeeding period, which continued despite restoration of pre-restriction body weight. Data are expressed as mean+SD. All data were analyzed via a two-way ANOVA with Bonferroni's multiple comparison tests. Days marked with * represents significant differences between CR and Ad-lib groups (p-values <0.05). n=52 per group days 1-17, n=35 per group days 18-19, and n=22 per group on day 20.

FIG. 12 shows temporal changes in absolute fat mass, lean mass, and body composition following refeeding after 60% caloric restriction. Six days of 60% caloric restriction in male mice led to significant reductions in both fat mass and lean mass when quantified as either % of body weight (Day 0, panels A and B,) or absolute change in mass (Day 0, panels C and D). The effect on fat mass persisted during the first two days of the refeeding period (Day 1 and 2, panels A and C). In terms of overall body composition, this reduction in fat mass represented a 50% reduction in % fat mass relative to ad libitum fed (Ad-lib) controls (Day 0, panel A). This dramatic reduction in % fat mass led to CR mice having significantly higher % lean mass on the final day of caloric restriction relative to Ad-lib mice (Day 0, panel B), although total lean mass was significantly reduced relative to Ab-lib mice at the same time point (panel D), and this effect reversed on the first day of the refeeding period (Day 1, panel B). % lean mass of CR mice equalized relative to Ad-lib mice on the second and fourth day of the refeeding period (Days 2 and 4, panel B), while total lean mass continued to be reduced in CR mice on the second day of refeeding before equalizing on the fourth day (Days 2 and 4, panel D). Data are expressed as mean+SD. Data were analyzed via two-way ANOVA. Group differences on each Day were determined via Bonferroni's multiple comparison test with significance levels as follows: ***-p<0.001. n=52 per group baseline, Day 0 and Day 1, n=35 per group Day 2, and n=21 per group on Day 4.

FIG. 13 shows temporal changes in hormones and metabolites following refeeding after 60% caloric restriction in male mice. Six days of 60% caloric restriction in mice led to significant reductions in fasting glucose, leptin, and hepatic glycogen levels (Day 0, Panels A, B, and C), while significantly increasing fasting ghrelin and β-hydroxybutyrate levels (Day 0, Panels D and E). Following a single day of refeeding, levels of hepatic glycogen, ghrelin, and β-hydroxybutyrate normalized and were not significantly different from ad libitum fed control mice for the remainder of the refeeding period, with the exception of Day 4 hepatic glycogen (Day 1, 2, and 4, Panels C, D, and E). In contrast, fasting glucose and leptin remained significantly lower during the first day of refeeding in mice exposed to 60% caloric restriction before returning to levels similar to Ad-lib mice on Days 2 and 4 of the refeeding period (Panels A and B). Fasting glucagon levels were not significantly different between the two groups on the final day of caloric restriction (Day 0, Panel E), nor were there any differences between the groups during the refeeding period (Days 1, 2, and 4, Panel E). Data are expressed as mean+SD. Data were analyzed via two-way ANOVA. Group differences on each day were determined via Bonferroni's multiple comparison test with significance levels as follows: *-p<0.05; **-p<0.01; ***-p<0.001. n=7-8/group (A and B); n=5-8/group (D, E, and F); n=4-6/group (C).

FIG. 14 shows hypoglycemic counterregulation is altered following exposure to six days of 60% caloric restriction. One, two, and four days following refeeding, hypoglycemia-induced glucagon secretion was assessed via a 60-minute hypoglycemic insulin tolerance test (A) in male mice exposed to a six-day 60% caloric restriction paradigm (CR, black) and ad libitum fed controls (Ad-lib, grey). In order to overcome differences in insulin sensitivity and fasting blood glucose levels between groups, a variable dose of insulin (IP) was used to produce equivalent exposure to hypoglycemia (B). Mice exposed to 60% caloric restriction displayed a significant reduction in hypoglycemia-stimulated glucagon release on Day 1 and a significant increase in glucagon release on Day 4 relative to control mice, while no between group differences occurred on Day 2 (C). Hypoglycemia-induced corticosterone levels were also reduced at the 60-minute time point one day following refeeding (D, p=0.034). Data are expressed as mean+SD, and were analyzed via two-way ANOVA or t-test (corticosterone data). Group differences on each day of refeeding were determined via Bonferroni's multiple comparison test with significance levels as follows: *-p<0.05; **-p<0.01; n=8-10/group (A and C), n=5-7/group (B), and 14-15/group (D).

FIG. 15 shows leptin treatment during caloric restriction restores normal hypoglycemia-induced glucagon release without reversing hypoleptinemia. Two groups of male mice were exposed to a six-day 60% caloric restriction paradigm and received either twice-daily leptin treatment (CR+Leptin, black semicircles) or PBS injections (CR+PBS, black circles) during the CR paradigm. A third group consisted of ad libitum fed control animals (Ad-lib, grey squares). Free-living blood glucose levels during the period of caloric restriction were similar in CR+PBS and CR+Leptin mice, but reduced in both groups relative to Ad-lib mice on the last five days of restriction (A). One day following refeeding, counterregulatory hormone levels were assessed via a 60-minute insulin tolerance test (ITT) in all three groups (B). In order to overcome differences in insulin sensitivity and fasting blood glucose levels between the control and CR groups, a variable dose of insulin (IP) was used to produce equivalent exposure to hypoglycemia (C). Hypoglycemia-induced glucagon levels were significantly reduced in the PBS treated CR mice relative to ad-lib mice, while leptin-treated CR mice showed no deficit (D). There were no differences in corticosterone levels between the three groups at the 60-minute time point of the ITT (E), but leptin levels were significantly reduced in both CR+PBS and CR+Leptin mice at this time point relative to ad-lib mice (F). Data are expressed as mean+SD. Data were analyzed via two-way ANOVA (A and B) or one-way ANOVA (C-F) and group differences were determined via Bonferroni's multiple comparison test relative to control mice. *- p<0.05.

FIG. 16 shows leptin treatment during exposure to three days of recurrent hypoglycemia restores normal hypoglycemia-induced epinephrine levels in male rats. Counterregulatory hormone and leptin levels were assessed following a 60-minute hypoglycemic insulin tolerance test (ITT) in three groups of rats. Two groups were exposed to three days of recurrent hypoglycemia (3dRH) via IP insulin and received either daily leptin (3dRH-Leptin, black semicircles) or PBS injections (3dRH-PBS, black circles) during the 3dRH paradigm. One group received neither leptin nor insulin (Control, grey squares). Glucose levels of the three groups during both the 3dRH paradigm and the hypoglycemic ITT are shown in Panel A. Hypoglycemia-induced epinephrine levels were significantly reduced in the 3dRH-PBS rats relative to controls, while 3dRH-Leptin rats showed no deficit (B). There were no differences in corticosterone levels between the three groups at the 60-minute time point of the ITT (C). Leptin levels at the 60-minute time point in 3dRH-PBS rats were not different from control rats, while leptin levels of 3dRH-Leptin rats were significantly increased (D). Data were analyzed via two-way ANOVA and group differences were determined via Bonferroni's multiple comparison test relative to control rats. *- p<0.05.

FIG. 17 shows six days of 60% caloric restriction leads to significant reductions in body weight and blood glucose, and hypoglycemia-induced glucagon release follow refeeding in female mice. Following a five-day acclimation period and six days of baseline food intake data, wild type B6 female mice were assigned to either a 60% caloric restriction paradigm (CR mice; black circles) or ad libitum access to chow (Ad-lib mice; grey squares) for six days. Following the caloric restriction period, both groups of mice, CR and Ad-lib, were given ad libitum access to chow for 16 h (see top border for timing of each experimental period). CR mice experienced significant reductions in both body weight and blood glucose during the CR paradigm with both measurements reaching their nadir on the final day of the paradigm. One day following refeeding, hypoglycemia-induced glucagon secretion was assessed via a 60-minute hypoglycemic insulin tolerance test (A). In order to overcome differences in insulin sensitivity and fasting blood glucose levels between groups, a variable dose of insulin (IP) was used to produce equivalent exposure to hypoglycemia (B). Mice exposed to 60% caloric restriction displayed a significant reduction in hypoglycemia-stimulated glucagon release relative to controls Data are expressed as mean+SD. All data were analyzed via a two-way ANOVA with Bonferroni's multiple comparisons tests or t-tests (insulin dose and glucagon). Days marked with * represents significant differences between CR and Ad-lib groups (p-values <0.05). n=4 per group

FIG. 18 shows serum insulin levels at the end of a hypoglycemic insulin tolerance test (ITT) in experimental and control mice exposed to severe caloric restriction were similar and highly correlated with the dose of insulin administered. One, two, and four days following refeeding, serum insulin levels were measured at the conclusion (60 min time point) of a hypoglycemic ITT (A) in male mice exposed to a six-day 60% caloric restriction paradigm (CR, black) and ad libitum fed controls (Ad-lib, grey). In order to overcome differences in insulin sensitivity and fasting blood glucose levels, both between and within groups, a variable dose of insulin (1.25-2.33 U/kg, IP) was used to produce equivalent exposure to hypoglycemia, ≈50 mg/dL for 30 min. Despite this large range of doses, there was no difference in serum insulin levels between CR and Ab-lib mice following the ITT on Days 2 and 4 of the refeeding period, although serum insulin levels were lower in CR mice relative to Ad-lib controls following the ITT on Day 1 (p=0.005). Correlational analysis demonstrated a significant linear relationship between insulin dose and serum insulin levels following the ITT (B) and indicates that insulin dose strongly predicts serum insulin levels at the 60 min time point of the ITT (p<0.001, r2=0.431). Data in (A) are expressed as mean+SD, and were analyzed via two-way ANOVA. Group differences on each day of refeeding were determined via Bonferroni's multiple comparison test. **-p<0.01; n=6-7/group. Data in (B) represent paired values of insulin dose and serum levels plotted for all data in (A); n=37 (17 Ad-lib mice and 20 CR mice). P-values and r-squared values along with curve fits (black line), were determined by simple linear regression analysis.

FIG. 19 shows leptin levels following a hypoglycemic insulin tolerance test (ITT) were positively correlated with hypoglycemia-induced counterregulatory hormone levels in male mice exposed to 60% caloric restriction (CR), but not in male rats exposed to 3 days of recurrent hypoglycemia (3dRH). Concurrent measurement of serum leptin and glucagon (mice) or epinephrine (rats) were made 60 min following the start of a hypoglycemic ITT (insulin dose=1.6-2.3 U/kg in mice, 10 U/kg in rats). Following one day of refeeding, hypoglycemia-induced glucagon levels were significantly correlated with leptin levels in mice exposed to CR (A, black circles, p=0.03, r2=0.21). Glucagon and leptin levels were not correlated in ad-libitum fed control mice (A, grey squares) or in mice exposed to CR which received twice daily leptin injections during the CR paradigm (A, black semi circles). In contrast, leptin and epinephrine levels were not correlated in control rats (B, grey squares), rats exposed to 3dRH (B, black circles), or rats exposed to 3dRH which received daily leptin injections during the 3dRH paradigm (B, black semi circles). Best linear fits to each of the three data sets: control, CR/3dRH alone, CR/3dRH plus leptin, are represented by grey, black, and dotted lines respectively in panels A and B. n=10-22/group in panel A and n=6/group in panel B. P-values and r-squared values along with curve fits (solid and dashed lines), were determined by simple linear regression analysis, and the values of these parameters for each group can be found in the figure legend

FIG. 20 shows the overview of clinical study design, measurements, outcome measures, and timeline of visits.

FIG. 21 shows the schedule of assessments.

FIG. 22 shows the schedule of measurements during clamps.

FIG. 23 shows non-limiting examples of complications of severe hypoglycemia, complications of diabetes, and blood glucose ranges. Adapted from UKPDS Group. Diabetes Res 1990: 13(1):1-11; Fong D S et al. Diabetes Care 2003: 26(Supp 1): S99-S102; HSD. J Hypertens 1993: 11(3): 309-371; Molitch M E et al. Diabetes Care 2003: 26(Supp 1): S94-S98; Kannel W B et al. Am Heart J 1990: 120:672-676. 6. Gray R P et al. In Textbook of Diabetes 2nd Edition, 1997. 7. Kings Fund. London: British Diabetic Association, 1996. 8. Mayfield J A et al. Diabetes Care 2003: 26(Supp 1): S78-S79.

FIG. 24 shows a schematic indicating that properly dosing insulin is difficult and errors have significant impacts on health. Adapted from UKPDS Group. Diabetes Res 1990: 13(1):1-11; Fong D S et al. Diabetes Care 2003: 26(Supp 1): S99-S102; HSD. J Hypertens 1993: 11(3): 309-371; Molitch M E et al. Diabetes Care 2003: 26(Supp 1): S94-S98; Kannel W B et al. Am Heart J 1990: 120:672-676; Gray R P et al. In Textbook of Diabetes 2nd Edition, 1997; Kings Fund. London: British Diabetic Association, 1996; Mayfield J A et al. Diabetes Care 2003: 26(Supp 1): S78-S79.

FIG. 25 shows a schematic indicating that properly dosing insulin is difficult and errors have significant impacts on health. Adapted from UKPDS Group. Diabetes Res 1990: 13(1):1-11; Fong D S et al. Diabetes Care 2003: 26(Supp 1): S99-S102; HSD. J Hypertens 1993: 11(3): 309-371; Molitch M E et al. Diabetes Care 2003: 26(Supp 1): S94-S98; Kannel W B et al. Am Heart J 1990: 120:672-676; Gray R P et al. In Textbook of Diabetes 2nd Edition, 1997; Kings Fund. London: British Diabetic Association, 1996; Mayfield J A et al. Diabetes Care 2003: 26(Supp 1): S78-S79.

FIG. 26 shows a schematic indicating that properly dosing insulin is difficult and errors have significant impacts on health. Adapted from UKPDS Group. Diabetes Res 1990: 13(1):1-11; Fong D S et al. Diabetes Care 2003: 26(Supp 1): S99-S102; HSD. J Hypertens 1993: 11(3): 309-371; Molitch M E et al. Diabetes Care 2003: 26(Supp 1): S94-S98; Kannel W B et al. Am Heart J 1990: 120:672-676; Gray R P et al. In Textbook of Diabetes 2nd Edition, 1997; Kings Fund. London: British Diabetic Association, 1996; Mayfield J A et al. Diabetes Care 2003: 26(Supp 1): S78-S79.

FIG. 27 shows a depiction of hypoglycemia associated autonomic failure (HAAF). Adapted from Cryer, P E and Childs, B P (2002). Diabetes Spectrum 15(1):20-27.

FIG. 28 shows the measurement of glucose counter-regulation with the hyperinsulinemic-hypoglyemic clamp and measurement of stress hormones.

FIG. 29 shows the results from an experiment where participants were exposed to 4 bouts of hypoglycemia over 5 days using the hypoglycemic clamp technique. (Adapted from Sequist lab)

FIG. 30 shows data regarding plasma glucose concentrations, plasma epinephrine concentrations, and plasma glucagon concentrations.

FIG. 31 shows a depiction of exemplary mechanisms underlying HAAF.

FIG. 32 shows that alterations in glial physiology is a target for the prevention and treatment of hypoglycemic complications.

FIG. 33 shows cross-sectional evidence that glial acetate metabolism and the neuroendocrine response to hypoglycemia are closely associated when the two are measured simultaneously.

FIG. 34 shows an overview of the study from McDougal et al. (McDougal, D H, et al. Acta diabetologica 55(10): 1029-1036 PMCID: 6153507)

FIG. 35 shows levels of beta-hydroxybutyratem, glucose, free fatty acids and acetoacetate during days of fasting (adapted from Cahill, G F, Jr. (1983) Transactions of the American Clinical and Clim. Assoc. 94: 1-21).

FIG. 36 shows a depiction of the association of starvation with increased serum levels of ketone bodies, which are chemically similar to acetate.

FIG. 37 shows a schematic exemplifying HAAF and starvation.

FIG. 38 shows an experimental design for HAAF and starvation.

FIG. 39 shows mice fed a KD, either chronically or acutely, experienced ketosis.

FIG. 40 shows glucagon and the change in blood glucagon levels with chronic exposure to KD.

FIG. 41 shows glucagon and the change in blood glucagon levels when mice are acutely exposed to KD.

FIG. 42 shows a depiction of ketogenic diets as an increasingly popular secondary treatment for diabetes.

FIG. 43 shows a depiction of exemplary effects of a ketogenic diet.

FIG. 44 shows data from Adamson et al. on the effects of 72 hours of fasting leading to HAAF symptoms. (adapted from Adamson, U. et al., Scandinavian Journal of clinical and laboratory investigation, 1989, 49(8): p. 751-6)

FIG. 45 is a schematic showing an experimental design validating an embodiment of the invention.

FIG. 46 shows the effects of 60% caloric restriction on body weight and blood glucagon levels.

FIG. 47 shows levels of fasting glucose and leptin levels.

FIG. 48 shows levels of glucagon, ketones, and helptic glycogen levels.

FIG. 49 shows leptin levels to days relative to refeedings.

FIG. 50 shows a schematic of the effects of HAAF and starvation.

FIG. 51 shows graphs of blood glucose levels and epinephrine levels in health individuals and individuals with anorexia nervosa. (Adapted from Nakagawa, K., et al (1985) Endocrinologia japonica 32(5): 719-724.

FIG. 52 shows a graph adapted from Fuji, S., et al (1989) Acta endocrinologica 120(5): 610-615.

FIG. 53 shows a graph of leptin levels (Adapted from Sandoval, D A et al. (2003), Journal of Diabetes and Its Complications 17(6): 301-306.

FIG. 54 shows plasma glucose and plasma epinephrine levels (Adapted from Osundiji, M A, et al. (2011) Metabolism: clinical and experimental 60(4): 550-556. from Osundiji et al.

FIG. 55 shows a graph of basal leptin levels (Adapted from Reno, C M et al (2015) American Journal of Physiology. Endocrinology and Metabolism 309 (12): E960-967.

FIG. 56 shows glucagon and epinephrine levels (Adapted from Reno, C M et al (2015) American Journal of Physiology. Endocrinology and Metabolism 309 (12): E960-967).

FIG. 57 shows a graph of leptin levels to days relative to refeeding.

FIG. 58 shows an overview of Aim 1.

FIG. 59 shows a protocol for Aim 2.

FIG. 60 shows results from Aim 2.

FIG. 61 shows experimental design from Aim 2.

FIG. 62 show a depiction of translational research.

FIG. 63 shows a depiction of the translation of lab research to implementation. Adapted from Gibbons, Gary. “CHALLENGES AND OPPORTUNITIES IN RESEARCH AND TRANSLATION.” Exploring Strategies to Improve Cardiac Arrest Survival: Proceedings of a Workshop. National Academies Press, 2017.

FIG. 64 shows changes in blood glucose over a specified time course.

FIG. 65 shows the study design and timing of study visits.

FIG. 66 shows a study design.

### DETAILED DESCRIPTION OF THE INVENTION

Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

Hypoglycemia

“Hypoglycemia” can refer to decreased levels of glucose in plasma, or below normal levels. Although hypoglycemic subjects can be asymptomatic, many exhibit adrenergic stimulation symptoms such as diaphoresis, anxiety, irritability, palpitations, tremor, and hunger. Hypoglycemic events can also occur during the night-time (nocturnal hypoglycemia), for example, when a person is sleeping, thus the subject is vulnerable to continuing decreases in levels of glucose in plasma.

Some individuals can experience recurrent bouts of severe hypoglycemia. Clinically, a subject with hypoglycemia has a blood glucose level of equal to or less than 70 mg/dl, whereas a subject can be classified as being severely hypoglycemic when the subject has a blood glucose level of less than 54 mg/dL, such as less than 50 mg/dL. Because such episodes of hypoglycemia can cause severe complications, it is recommended that individuals with a recent history of severe hypoglycemia better recognize the occurrence of low blood glucose. Severe hypoglycemia can cause confusion, visual blurring, loss of consciousness and seizures.

Recurrent bouts of hypoglycemia in patients with diabetes often lead to the development of hypoglycemia associated autonomic failure (HAAF). HAAF is a serious condition characterized by drastically reduced neuroendocrine responses to hypoglycemia as well as the loss of the physiological symptoms of hypoglycemia, or hypoglycemia unawareness. The development of HAAF can lead to ever worsening, and often life-threatening, episodes of severe hypoglycemia.

The typical insulin dependent diabetic patient will experience one episode of severe hypoglycemia per year, often involving loss of consciousness or seizure. The rate of severe hypoglycemic episodes in patients who are insulin dependent for greater than 15 yrs rises to three per year. Severe hypoglycemia is associated with a 3.4-fold increase risk of death in diabetic patients, and long-term longitudinal studies indicate that 6-10% of individuals with type 1 diabetes die as a result of acute hypoglycemia. A majority of these deaths are influenced by the development of HAAF.

The etiology of hypoglycemia is often idiopathic, but can be caused by early diabetes, fasting, malignancies of the pancreas, benign tumors of the pancreas, general hypertrophy of the pancreas without evident disease, alcohol intake and liver disease (decreased gluconeogenesis), gastrectomy, renal failure, drugs such as salicylates, beta-blockers, pentamidine, acetylcholine esterase (ACE) inhibitors, excess insulin including insulinoma, self-administered insulin or oral hypoglycemic agents; pituitary or adrenal insufficiency.

In addition, fasting hypoglycemia is a category of hypoglycemia that clinically can have symptoms of neuroglycopenia, including headache, fatigue, and mental dullness. In more severe cases, hypoglycemia can progress to confusion, blurring of vision, seizure, and ultimately loss of consciousness or seizure. Fasting hypoglycemia can occur with a fast of greater than 4 hours, and further can be caused by hyperinsulinemia (resulting from self-administered insulin or intake of other hypoglycemic agents), alcohol abuse, liver disease (e.g., decreased gluconeogenesis), pituitary insufficiency, or adrenal insufficiency.

Subjects are often ordered by a healthcare professional to fast prior to or for a medical test. For some medical tests, fasting beforehand gives a more accurate result. For other tests or operations, you need to fast for safety reasons. “Fasting” or “to fast” refers to not eating and only drinking sips of water for a period of time, such as overnight or for a period of hours. A subject who is fasting can't drink fruit juice, soft drink, coffee, tea or milk, and can't eat or suck on lollies and chewing gum. A fasting blood test can be done after a subject has fasted for about 8 to 16 hours, such as in the morning. A subject can fast for about 6 hours before a gastroscopy, so as to lower the risk of vomiting up and inhaling what's in the subject's stomach, and to also gives the physician a clear view inside the stomach and intestine. A subject can be asked to fast before a colonoscopy, such as overnight. A physician can ask a subject being sedated or having a general anesthetic to fast for several hours beforehand. Subjects who have diabetes or other metabolic disorders, for example Addison's disease, congenital hypopituitarism, or gastric dumping syndrome (such as after gastric bypass surgery), are susceptible to hypoglycemia and hypoglycemic-complications as a result of fasting.

Hypoglycemic complications are a major barrier to the successful treatment of individual's with diabetes. Paradoxically, repeated bouts of severe-hypoglycemia leads to the loss of the normal physiological response to hypoglycemia, called the counter-regulatory response. In some embodiments, hypoglycemia-associated complication comprises hypoglycemia associated autonomic failure (HAAF), a cardiac condition, a neurological condition, or a combination thereof. Non-limiting examples of a cardiac condition comprises cardiac arrhythmia or cardiac arrest. Non-limiting examples of a neurological condition comprises confusion, seizure, loss of consciousness, coma, brain death, or a combination thereof.

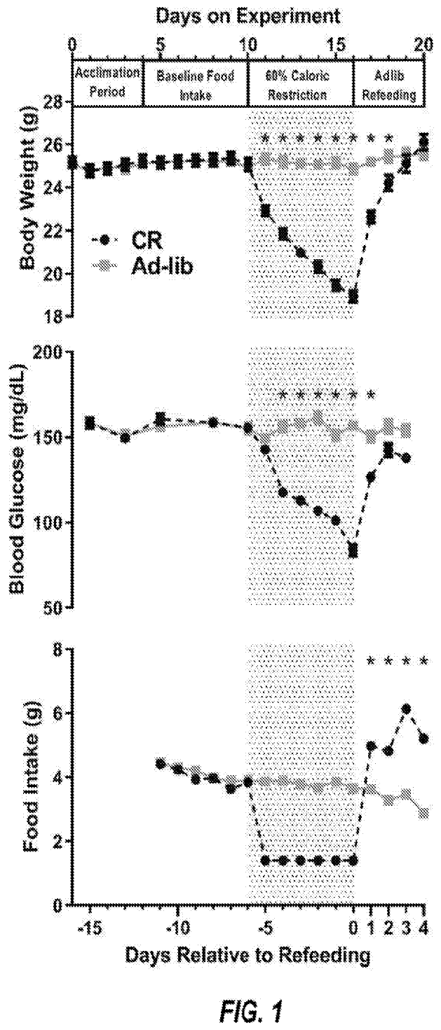

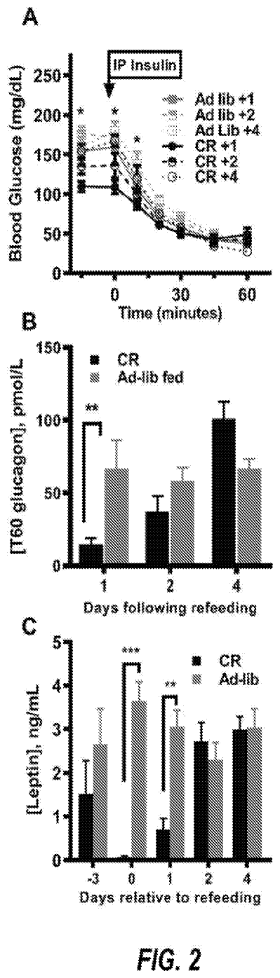

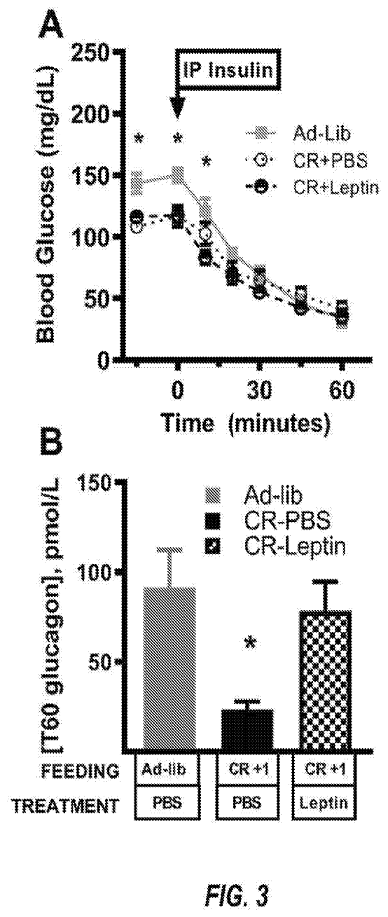

We have recently completed a series of experiments which indicate that this paradoxical loss of counter-regulation may be driven by exposure to hypoleptinemia, or severely reduced blood leptin levels. Leptin is a hormone released by adipose tissue that acts as a signal of energy status, which affects food intake and the neuroendocrine system. Our experiments utilized six days of 60% caloric restriction to induce a starvation response in the mouse model (FIG. 1). One day following refeeding, calorically restricted mice displayed a deficit in the counter-regulatory response, indicated by significantly reduced hypoglycemia-induced glucagon release, relative to ad libitum (Ad-lib) fed control mice not exposed to caloric restriction (FIG. 2, panel A and panel B). This phenomenon was preceded by a drastic reduction in leptin levels in calorically restricted mice, which began three days prior to refeeding and remained through the first day of refeeding (see Day −3, 0, and 1 in FIG. 2C). More significantly, the caloric restriction-induced loss of counter-regulation was completely reversed by preventing the drop in leptin levels via twice-daily exogenous leptin treatment (FIG. 3, panel A and panel B).

Prior work in the field has demonstrated that recurrent exposure to hypoglycemia, which is known to cause a loss of the counter-regulatory response, leads to hypoleptinemia in rats (Reno, C M et al. 2015. American journal of physiology. Endocrinology and metabolism, 2015. 309(12): p. E960-7). We are currently testing whether a similar effect is present in the mouse model. Although Reno et al. demonstrated that acute administration of leptin did not restore counter-regulation, our results in the caloric restriction model indicate that preventing the hypoleptinemic state, as opposed to acute leptin administration, is required to reverse the effect. Thus, hypoleptinemia itself is not the direct cause of the paradoxical loss of counter-regulation following recurrent exposure to hypoglycemia, but that it drives the underlying physiological adaptations that cause the effect. In this way, prevention of the hypoleptinemic state, via longer-term leptin supplementation, is critical to prevent the underlying cause of the loss of the counter-regulatory response following recurrent exposure to hypoglycemia. This also indicates that a multi-day course of exogenous leptin treatment might induce a reversal of the loss of counter-regulation induced by recurrent exposure to hypoglycemia. Therefore, without wishing to be bound by theory, exogenous leptin treatment in humans can be effective in preventing or reversing the loss of counter-regulation in individuals with diabetes, and thus improving the efficacy their diabetes treatment plan and improving their health outcomes.

### EMBODIMENTS OF THE INVENTION

Aspects of the invention are drawn to methods of treating or preventing complications associated with hypoglycemia, the method comprising administering to a subject in need thereof a therapeutically effective amount of a composition comprising leptin and/or a leptin receptor agonist prior to/at onset of severe hypoglycemia.

Exposure to severe hypoglycemia is associated with increased risk of cardiac arrhythmias, sudden cardiac arrest, as well as serious neurological complications such as confusion, seizure, loss of consciousness, coma, and brain death. Severe hypoglycemia can be experienced during prolonged fasting or during strenuous exercise, although it can also be experienced as a result of the treatment of diabetes.

These treatment-induced hypoglycemic episodes are a significant impediment to the maintenance of healthy glucose levels in individuals with diabetes. The typical insulin dependent diabetic patient will experience one episode of severe hypoglycemia per year, often involving loss of consciousness or seizure. The rate of severe hypoglycemic episodes in patients who are insulin dependent for greater than 15 years rises to three per year. Severe hypoglycemia is associated with a 3.4-fold increase risk of death in diabetic patients, and long-term longitudinal studies indicate that 6-10% of individuals with type 1 diabetes die as a result of acute hypoglycemia.

Furthermore, recurrent bouts of hypoglycemia in patients with diabetes often lead to the development of hypoglycemia associated autonomic failure (HAAF). HAAF is a serious condition characterized by drastically reduced neuroendocrine responses to hypoglycemia as well as the loss of the physiological symptoms of hypoglycemia, or hypoglycemia unawareness. The development of HAAF can lead to ever worsening, and often life-threatening, episodes of severe hypoglycemia.

The term “treating” can refer to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition. For example, “treating” can refer to treating complications associated with hypoglycemia or severe hypoglycemia. In some embodiments, hypoglycemia-associated complication comprises hypoglycemia associated autonomic failure (HAAF), a cardiac condition, a neurological condition, or a combination thereof. Non-limiting examples of a cardiac condition comprises cardiac arrhythmia or cardiac arrest. Non-limiting examples of a neurological condition comprises confusion, seizure, loss of consciousness, coma, brain death, or a combination thereof.

Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition. In some embodiments, treatment comprises administering to a subject in need thereof a therapeutically effective amount of a composition comprising leptin.

In embodiments, a subject can be administered a composition described herein prior to the onset of severe hypoglycemia, and thus prevent the development of a condition or disease. The subject at risk of hypoglycemia can be identified by, for example, prior history of severe hypoglycemia, or increased time spent below a blood glucose of 70 mg/dL as recorded by continuous glucose monitoring or self-reported based on home glucose monitoring. The term “prevention” or “preventing” can refer to a stop or obstacle to the development of the disease, disorder or symptom of a disease or condition through some action. For example, conditions and symptoms as described herein can be prevented by administering compositions comprising leptin as described herein.

Hypoglycemia associated disorders and complications related to hypoglycemia can refer to conditions or complications caused by low blood sugar. Hypoglycemia can be associated with medical conditions, such as diabetes and/or insulinoma.

The term “therapeutic composition” can refer to any compounds administered to treat or prevent a disease or a symptom(s) thereof, such as complications associated with hypoglycemia. For example, aspects of the invention are drawn towards uses of therapeutic compositions comprising leptin and/or leptin receptor agonists.

Leptin is a hormone secreted by adipocytes that has been extensively studied for its role on food intake and body weight. This gene encodes a protein that is secreted by white adipocytes into the circulation and plays a major role in the regulation of energy homeostasis. Circulating leptin binds to the leptin receptor in the brain, which activates downstream signaling pathways that inhibit feeding and promote energy expenditure. This protein also has several endocrine functions, and is involved in the regulation of immune and inflammatory responses, hematopoiesis, angiogenesis, reproduction, bone formation and wound healing. Mutations in this gene and its regulatory regions cause severe obesity and morbid obesity with hypogonadism in human patients. A mutation in this gene has also been linked to type 2 diabetes mellitus development.

The amino acid sequence of the leptin precursor protein (NCBI Ref. Seq: NP_000221.1; length: 167 amino acids; 16,641 Mass (Da); SEQ ID NO: ______) comprises:

1 mhwgticgfl wlwpylfyvq avpiqkvqdd tktliktivt rindishtqs vsskqkvtgl

61 dfipglhpil tlskmdqtla vyqqiltsmp srnviqisnd lenlrdllhv lafskschlp

121 wasgletlds lggvleasgy stevvalsrl qgslqdmlwq ldlspgc

As used in compositions described herein, “leptin” and “leptin receptor agonists” can be used interchangeably. A “leptin receptor agonist” can refer to an agent acting on the leptin receptor. See, for example, Roujeau, Clara, Ralf Jockers, and Julie Dam. “New pharmacological perspectives for the leptin receptor in the treatment of obesity.” Frontiers in endocrinology 5 (2014): 167, which describes stabilized leptin derivatives and small synthetic agonists.

In embodiments, the therapeutic composition can comprise human recombinant leptin and derivatives or fragments thereof. For example, Metreleptin (Myalept) is an FDA-approved treatment for generalized and familial dyslipidemia, and thus could readily be repurposed to treat or prevent the loss of the counter-regulatory response in diabetes patients. Thus, embodiments of the invention comprise treatment strategies for utilizing Metreleptin to treat or prevent loss of counter-regulation.

Compositions as utilized herein can also be provided as therapeutic or prophylactic combination compositions that comprise leptin, fragments thereof, and/or leptin receptor agonists, and one or more additional active agents. For example, a therapeutic or prophylactic combination composition can comprise leptin and insulin that can be used to prevent and/or treat hypoleptinemia and improve insulin sensitivity. As another example, the therapeutic or prophylactic combination composition can comprise leptin and insulin secretagogues, such as sulfonylureas and glinides.

The term “combination” or “combination composition” can refer to either a fixed combination in one dosage unit form, or a kit of parts for the combined administration where a compound and a combination partner (e.g., another drug, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

The therapeutic compositions can be incorporated into pharmaceutical compositions suitable for administration. Such compositions can comprise leptin, fragments thereof, and/or leptin receptor agonists, and a pharmaceutically acceptable carrier. Thus, in some embodiments, the compounds of the invention are present in a pharmaceutical composition. According to the invention, a pharmaceutically acceptable carrier can comprise any and all solvents, dispersion media, coatings, antibacterial and antifungal agents, isotonic and absorption delaying agents, and the like, compatible with pharmaceutical administration. Non-limiting examples of pharmaceutically acceptable carriers comprise solid or liquid fillers, diluents, and encapsulating substances, including but not limited to lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil. The use of such media and agents for pharmaceutically active substances is well known in the art. Any conventional media or agent that is compatible with the active compound can be used. Supplementary active compounds can also be incorporated into the compositions.

The term “therapeutically effective amount” can refer to those amounts that, when administered to a subject in view of the nature and severity of that subject's disease or condition, will have a desired therapeutic effect, e.g., an amount which will cure, prevent, inhibit, or at least partially arrest or partially prevent a target disease or condition. In some embodiments, the term “therapeutically effective amount” or “effective amount” can refer to an amount of a therapeutic agent that when administered alone or in combination with an additional therapeutic agent to a cell, tissue, or subject is effective to prevent or ameliorate the disease or condition such as a hypoglycemia-associated disease or condition or the progression of the disease or condition. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

A therapeutically effective dose can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner desires. These amounts can be readily determined by the skilled artisan.

In some embodiments, the therapeutically effective amount is at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. The dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

For example, the therapeutically effective amount can refer to an amount of leptin, fragment thereof, or leptin receptor agonists. For example, the therapeutically effective amount can refer to an amount of insulin or insulin secretagogues.

In embodiments, the insulin dose can be adjusted. See, for example, Vasandani et al (2017) Diabetes Care, 40:694-697; DOI: 10.2337/dc16-1553. For example, the insulin dose can be adjusted up or adjusted down. For example, the insulin dose can be adjusted up for a period of time, and then down for a period of time. In a clinical setting, it can be important to titrate the dosage of insulin to safely treat the patient. For example, the capillary blood glucose values can be monitored, and dose adjustments can be made to avoid hypo- and/or hyperglycemia. For example, insulin dose can be reduced if preprandial blood glucose is about <80 mg/dL or the postprandial value is about <99 mg/dL. In another example, insulin dose can be increased if preprandial blood glucose is about >200 mg/dL or the postprandial value is about >250 mg/dL.

In embodiments, the leptin dose can be adjusted. For example, the leptin dose can be adjusted up or adjusted down. For example, the leptin dose can be adjusted up for a period of time, and then down for a period of time. In a clinical setting, for example, leptin can be administered at a dose of about 0.04 mg/kg/day for female patients and 0.02 mg/kg/day for male patients for the first four weeks, and doubled after for an additional 16 weeks.

In embodiments, the insulin dose can be reduced once leptin treatment was started. For example, the insulin dose can be reduced by about 1%, about 2.5%, about 5%, about 10%, about 11%, about 12%, about 13%, about 14%, about 15%, about 20%, about 50%, about 75%, about 90%, about 99% or by 100%.

“Insulin” can refer to any form of insulin suitable for administration to a mammal, and includes insulin isolated from a mammal, recombinant insulin, insulin bound to or converted by other molecules, and modified insulin molecules, provided that they retain clinically significant activity lowering blood glucose levels. Also included are insulin compositions suitable for administration by any route, including pulmonary, subcutaneous, nasal, oral, buccal and sublingual. Insulin compositions can be prepared as dry powders, aqueous solutions or suspensions, or non-aqueous solutions or suspensions (which is typical of metered-dose inhalers) for inhalation; aqueous solutions or suspensions for subcutaneous, sublingual, buccal, nasal or oral administration; and solid dosage forms for oral and sublingual administration.

Insulin can include fast-acting insulin, medium-acting insulin, and long-acting insulin.

“Fast-acting insulin” can refer to to an insulin formulation that reaches a maximum concentration in the blood within about 45-90 minutes, and a maximum activity of about one to 3 hours after administration. Fast-acting insulin can remain active for approximately four to six hours. A non-limiting example of fast-acting insulin is the Lyspro insulin analogue (HUMALOG®).

“Medium-acting insulin” can refer to insulin with onset of action, approximately two to four hours after injection, and peaks from four to 12 hours after injection, and it continues to act for 10 to 18 hours. Typical medium-acting insulin preparations are prepared by mixing regular insulin with a substance that slows down the absorption of insulin. A non-limiting example is insulin NPH. Medium-acting insulin can provide many of the benefits of long-acting insulin.

“Long-acting insulin” can refer to a composition of insulin, which begins to work within about 1-6 hours and provides a constant level of insulin activity for up to 24 hours or more. Long-acting insulin acts with maximum strength after about 8-12 hours, sometimes longer. Long-acting insulin can be given in the morning and before bedtime. Non-limiting examples of long acting insulin include, but are not limited to, insulin glargine or insulin detemir, which are analogues of insulin, and ultralente insulin, which is a regular human insulin made for slow absorption. Long-acting insulin is best suited to meet the needs for basal, as opposed to prandial, insulin

Therapeutic compositions and combinations described herein, such as that comprising leptin, fragments thereof, and/or leptin receptor agonists, can be administered to the subject one time (e.g., as a single injection or deposition). Alternatively, administration can be once or twice daily to a subject in need thereof for a period of time, such as once or twice daily to a subject for a period of 1 week, 2 weeks, 3 weeks, 4 weeks, 12 weeks, 24 weeks, or 52 weeks per year, or longer.

Any of the therapeutic applications described herein can be administered to any subject in need of such therapy, such as a subject afflicted with a disease or condition associated with hypoglycemia or recurrent instances of hypoglycemia, or suffering from a hypoglycemia associated complication. Such subjects include, for example, a mammal such as a mouse, a rat, a dog, a cat, a cow, a horse, a rabbit, a monkey, a pig, a sheep, a goat, or a human. In some embodiments, the subject is a mouse, rat or human. In some embodiments, the subject is a mouse. In some embodiments, the subject is a rat. In some embodiments, the subject is a human. For example, the subject can be a diabetic subject experiencing moderate-to severe hypoglycemia, as recorded by self-monitored blood glucose levels or continuous glucose monitoring devices.

The term “administration” can refer to introducing a substance, such as leptin, fragments thereof, and/or leptin receptor agonists, or a composition comprising leptin, into a subject. Any route of administration may be utilized. Examples of routes of administration include parenteral, e.g., intravenous, intradermal, subcutaneous, oral, transdermal (topical), transmucosal, and rectal administration.

A pharmaceutical composition of the invention is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

In some embodiments, the methods described herein further comprise administering a composition comprising leptin as part of a treatment cycle. Thus, the treatment cycle may comprise administering the composition daily for 7, 14, 21, or 28 days, followed by 7 or 14 days without administration of the composition. In some embodiments, the treatment cycle comprises administering the amount of the composition daily for 7 days, followed by 7 days without administration of the composition. A treatment cycle may be repeated one or more times to provide a course of treatment. In addition, the composition may be administered once, twice, three times, or four times daily during the administration phase of the treatment cycle. In other embodiments, the methods further comprise administering the amount of the composition once, twice, three times, or four times daily or every other day during a course of treatment. A course of treatment can refer to a time period during which the subject undergoes treatment by the present methods. Thus, embodiments of the invention are directed towards administering to a subject in need thereof a short course (e.g., 7 to 14 days) of a composition comprising leptin. For example, the method comprises administering to a subject or prescribing to a subject a short course of a composition comprising leptin, such as human recombinant leptin, following treatment for severe hypoglycemia.

Embodiments of the invention are also directed towards the chronic administration of a composition comprising leptin, fragments thereof, and/or leptin receptor agonists, to a subject in need thereof. For example, the method can comprise chronic administration to a subject or prescribing to a subject chronic use of a composition comprising leptin, such as human recombinant leptin. For example a diabetic subject experiencing moderate-to-severe hypoglycemia may benefit from chronic administration of a composition comprising leptin.

The pharmaceutical compositions can also be provided in a kit. In one embodiment, the kit includes (a) a container that contains a composition that includes leptin, and optionally (b) informational material. The informational material can be descriptive, instructional, marketing or other material that relates to the methods described herein and/or the use of the agents for therapeutic benefit. In an embodiment, the kit includes also includes a second agent, such as insulin, for treating one or more complications associated with hypoglycemia. For example, the kit includes a first container that contains a composition that includes leptin, and a second container that includes the second agent, such as insulin.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the pharmaceutical composition, e.g., in a suitable dose, dosage form, or mode of administration (e.g., a dose, dosage form, or mode of administration described herein), to treat a subject who is afflicted with hypoglycemia-associated complications). The information can be provided in a variety of formats, include printed text, computer readable material, video recording, or audio recording, or an information that provides a link or address to substantive material.

In addition to leptin, the composition in the kit can include other ingredients, such as a solvent or buffer, a stabilizer, or a preservative. The composition can be provided in any form, e.g., liquid, dried or lyophilized form, for example substantially pure and/or sterile. When the composition is provided in a liquid solution, the liquid solution for example is an aqueous solution. When the composition is provided as a dried form, reconstitution is by the addition of a suitable solvent. The solvent, e.g., sterile water or buffer, can optionally be provided in the kit.

The kit can include one or more containers for the compositions. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the leptin and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Six Days of 60% Caloric Restriction Leads to Significant Reductions in Body Weight and Blood Glucose while Inducing Hyperphagia and Sustained Reductions in Blood Glucose Following Refeeding (Referring to FIG. 1, for Example)

Following a five-day acclimation period and six days of baseline food intake data, wild type B6 mice were assigned to either a 60% caloric restriction paradigm (CR mice; black circles) or ad libitum access to chow (Ad-lib mice; grey squares) for six days. Following the caloric restriction period, both groups of mice, CR and Ad-lib, were given ad libitum access to chow for four days (see top border for timing of each experimental period). CR mice experienced significant reductions in both body weight and blood glucose during the CR paradigm with both measurements reaching their nadir on the final day of the paradigm. The reduction in body weight was reversed within three days of refeeding, while blood glucose levels remained lower than those of Ad-lib mice following refeeding. CR mice also displayed a marked hyperphagia during the refeeding period, which continued despite restoration of pre-restriction body weight. All data were analyzed via a two-way ANOVA with Bonferroni's multiple comparisons tests. Days marked with * represents significant differences between CR and Ad-lib groups (p-values <0.05). n=52 per group days 1-17, n=35 per group days 18-19, and n=22 per group on day 20.

Hypoglycemic Counter-Regulation is Impaired Following Exposure to Six Days of 60% Caloric Restriction and is Preceded by Hypoleptinemia. Referring to FIG. 2, for Example

One, two, and four days (Referring to FIG. 2) following refeeding, hypoglycemic counterregulation was assessed via a 60-minute hypoglycemic insulin tolerance test (ITT) in mice exposed to a six-day 60% caloric restriction paradigm (CR, black) and ad libitum fed controls (Ad-lib, grey). In order to overcome differences in baseline blood glucose levels between groups, a variable dose of insulin (IP) was used to produce equivalent exposure to hypoglycemia (A). Following a single day of refeeding, mice exposed to 60% caloric restriction displayed a significant reduction in hypoglycemiastimulated glucagon release (Day 1, Panel B). There was no significant difference in hypoglycemia-stimulated glucagon release between caloric restriction and Ad-lib mice two and four days following refeeding (Day 2 and 4, Panel B). Six days of 60% caloric restriction in mice led to significant reductions in fasting leptin levels and leptin remained significantly lower during the first day of refeeding before returning to levels similar to Ad-lib mice on Days 2 and 4 of the refeeding period (Day 0 and 1, Panel C). Data were analyzed via two-way ANOVA. Group differences on each Day were determined via Bonferroni's multiple comparison test with significance levels as follows: *-p<0.05; **-p<0.01; ***-p<0.001. n=7-8/group.

Impaired Hypoglycemic Counterregulation Following Exposure to Six Days of 60% Caloric Restriction is Rescued by Leptin Treatment. Referring to FIG. 3, for Example.

One day following refeeding, hypoglycemic counter-regulation was assessed via a 60-minute hypoglycemic insulin tolerance test (ITT) in mice exposed to a six day 60% caloric restriction paradigm plus either twice daily injections of leptin (CRLeptin, checkered) or vehicle (CR-PBS, black) and ad libitum fed controls treated twice daily with vehicle (Ad-lib, grey). In order to overcome differences in baseline blood glucose levels between groups, a variable dose of insulin (IP) was used to produce equivalent exposure to hypoglycemia (A). Caloric restriction mice treated with vehicle had significant reduction in hypoglycemiastimulated glucagon release relative to Ad libitum fed controls, and leptin treatment during caloric restriction completely reversed this effect. Data were analyzed via one or two way ANOVA. Group differences were determined via Bonferroni's multiple comparison test with significance levels as follows: *- p<0.05; n=7-8/group.

### Example 2

This study sought to validate whether exposure to severe caloric restriction was sufficient to produce an altered neuroendocrine response to insulin-induced hypoglycemia in mice following reversal of the dietary restriction. Prior work has shown that severe caloric restriction is associated with abnormal hypoglycemic counter-regulation, yet it is unclear whether this effect persists following refeeding.

We demonstrate that mice previously exposed to severe caloric restriction display aberrant hypoglycemic counter-regulation following refeeding, which is coincident with hypoleptinemia and reduced fasting glucose levels. These findings provide insight into the underlying mechanisms driving loss of hypoglycemic counter-regulation in diabetic individuals on insulin therapy.

Abstract

Hypoglycemia-associated autonomic failure (HAAF) is a maladaptive failure in glucose counter-regulation known to be caused by recurrent exposure to insulin-induced hypoglycemia. Fasting or caloric restriction is the only natural physiological process which induces prolonged moderate-to-severe hypoglycemia similar to the antecedent of HAAF. In this study we tested whether exposure to severe caloric restriction can cause HAAF-like symptoms in mice. Mice exposed were placed on 60% caloric restriction for six consecutive days and then refed for up to four days (CR mice). Body weight, body composition, food intake, serum hormones and metabolites, as well as hepatic glycogen content and hypoglycemia-induced glucagon secretion were measured during caloric restriction and following refeeding. Mice exposed to six days of severe caloric restriction had significant reductions in body weight, fasting glucose, fat mass, hepatic glycogen content and leptin levels along with elevated ghrelin and β-hydroxybutyrate. Following refeeding, CR mice displayed defects in hypoglycemic counter-regulation, indicated by significantly lower hypoglycemia induced glucagon release relative to controls, 15.0+4.1 pmol/L versus 66.8+19.6 pmol/L, respectively (p=0.0087). This was accompanied by sustained reductions in leptin and fasting glucose during the refeeding period. In contrast, differences in ghrelin, β-hydroxybutyrate, and hepatic glycogen content were reversed within 24 hours of refeeding. Our results demonstrate that exposure to fasting-induced hypoglycemia produces HAAF-like symptoms in mice following refeeding. These results indicate that HAAF can be a result of an adaptive response to fasting which is inappropriately elicited during exposure to insulin-induced hypoglycemia.

Introduction

Patients with diabetes are vulnerable to hypoglycemia, and exposure to hypoglycemia is associated with an increased risk of all-cause mortality in these patients (Khunti et al., 2014). Patients with longstanding diabetes also often experience recurrent hypoglycemic episodes due to poorly regulated hyperinsulinemia and inadequate glucagon responses. Recurrent bouts of hypoglycemia lead to the development of hypoglycemia associated autonomic failure (HAAF) (Cryer, 2005, 2013a; Reno et al., 2013; Rogers et al., 2017; Lontchi-Yimagou et al., 2018), which is characterized by critically reduced neuroendocrine responses to hypoglycemia as well as hypoglycemia unawareness. Although the precise mechanisms underlying the development of HAAF are unknown, it is thought to be driven by CNS adaptations within the brain regions which directly detect reduced glucose availability (Beall et al., 2012; Taborsky & Mundinger, 2012; Cryer, 2013a; Reno et al., 2013; Lontchi-Yimagou et al., 2018).

Although HAAF is commonly experienced by diabetic patients on insulin therapy, diabetes is not required for its development. It is well established that HAAF-like symptoms can be induced in metabolically heathy humans and rodents via recurrent exposure to insulin-induced hypoglycemia (Moheet et al., 2014; Senthilkumaran et al., 2016). Thus, HAAF represents a pathophysiological adaptation brought about by repetitive exposure to hypoglycemia irrespective of disease state. Furthermore, the known antecedent of HAAF, insulin-induced hypoglycemia, is a relatively modern phenomenon which has only arisen following the adoption of exogenous insulin therapy as a treatment for diabetes. From an evolutionary perspective, this form of hypoglycemia would be exceedingly rare and unlikely to drive such distinct and ubiquitous physiological adaptations (Beall et al., 2012). In fact, glucose homeostasis is so well controlled in mammals that hypoglycemia is rarely experienced except during prolonged starvation (Goldstein et al., 2011; Watford, 2015).

The physiological responses to starvation are similar to those associated with HAAF and include changes in cerebral metabolism and physiology. As glucose stores are depleted during starvation, the brain transitions to relying almost exclusively on ketone bodies, monocarboxylate metabolites produced by the liver, to meet its energy requirements (Owen, 2005; Watford, 2015; Rojas-Morales et al., 2016). Starvation in humans has been shown to upregulate the cerebral metabolism of acetate (McDougal et al., 2018), an effect similar to that seen following diet-induced ketosis in humans (Bluml et al., 2002) and rodents (Melo et al., 2006). This cerebral adaptation is also strongly associated with the development of HAAF (Gulanski et al., 2013).

Starvation and its physiological antecedents are also associated with HAAF-like symptoms. The counter-regulatory response to insulin-induced hypoglycemia is significantly blunted directly following a 72-hour fast in humans (Adamson et al., 1989). Prolonged caloric restriction dramatically reduces hepatic glycogen content in both humans and animal models (Soeters et al., 2012; Watford, 2015), and depletion of hepatic glycogen, independent of fasting, leads to deficits in hypoglycemic counter-regulation in dogs (Winnick et al., 2016). Thus it is not clear whether starvation-induced deficits in hypoglycemic counter-regulation are due to fasting-induced metabolic adaptations or simply driven by significant reduction of hepatic glycogen stores caused by 72 hours of fasting. Furthermore, the fasted state itself induces mild activation of the counter-regulatory response, e.g. hyperglucagonemia, independent of overt hypoglycemia (Hojlund et al., 2001; McDougal et al., 2018). This phenomenon further complicates the study of starvation-induced changes in hypoglycemic counter-regulation.

In order to address this complication and to further test the association between starvation and the development of HAAF-like symptom, the current study was undertaken to determine if prolonged fasting can induce HAAF-like symptoms following refeeding. Assessment of hypoglycemic counter-regulation in the fed state eliminates the confounding influences of fasting-induced reductions in hepatic glycogen content and hyperglucagonemia. Furthermore, it is well established that the symptoms of HAAF peak 1-2 days following recurrent exposure to insulin-induced hypoglycemia (Moheet et al., 2014; Senthilkumaran et al., 2016). Thus if starvation is a natural antecedent to HAAF, HAAF-like symptoms should be manifest during post-starvation refeeding. Therefore, without wishing to be bound by theory, mice exposed to starvation would exhibit HAAF-like symptoms during acute refeeding. We utilized a well-established mouse model of starvation, 60% caloric restriction (Zhao et al., 2010; Goldstein et al., 2011; Mani et al., 2016), for six days and then assessed counter-regulation via a hypoglycemic insulin tolerance test (ITT) on the first, second, and fourth day of refeeding.

Methods

Animals

Male C57BL/6J wild-type mice 8-12 weeks of age (PBRC breeding colony) were single-housed in wire-bottomed cages with 12-hour light and 12-hour dark cycles, with the dark cycle beginning at either 18:00 or 19:00. All animals were fed Purina 5001 Rodent Laboratory Chow. Purified tap water in Hydropacs was provided ad libitum.

Severe Caloric Restriction and Refeeding Paradigm

Animals were assigned to one of two cohorts: Mice exposed to severe caloric restriction for six days (CR mice) or ad libitum fed (Ad-lib mice). See top border of FIG. 1 for graphical representation of the experimental timeline. Following a five day acclimation period to wire-bottomed cages (Days 0-4), food intake was monitored daily in each mouse for six days prior to initiation of caloric restriction and each mouse's average daily food intake was determined (Days 5-10). CR mice were then fed 40% of their daily average food intake for six days (Days 11-16) while Ad-lib mice continued to have unrestricted access to food. Mice were fed at ≈1.5 hours prior to lights off each day. Body weight and blood glucose (via hand held glucometer) were measured daily immediately prior to feeding. During the six days of CR, both CR and Ad-lib mice were administered 1.0 mL of warmed saline subcutaneously to prevent dehydration immediately following feeding or body weight and blood glucose measurements in CR and Ad-lib mice, respectively. After six days of restriction, the CR mice entered a re-feeding period and had ad libitum access to food for up to four days (Days 17-20). On Days 16, 17, 18, and 20 (FIG. 1, top x-axis), which represented the final day of CR and Days 1, 2, and 4 of the refeeding period (FIG. 1, bottom x-axis), a subset of CR and Ad-lib mice were euthanized for determination of blood hormones and metabolites as well as hepatic glycogen concentration. On Days 17, 18, and 20 separate cohorts of CR and Ad-lib mice underwent a hypoglycemic ITT for determination of intact hypoglycemic counter-regulation. During the refeeding period food intake, free-living blood glucose and body weight continued to be measured daily at ≈1.5 hours before lights off in both CR and Ad-lib mice up to the terminal day of the experiment.

Body Composition

Body composition was measured at baseline (Day 10), on the final day of caloric restriction (Day 16), and on the first, second, and fourth day of the refeeding period (Days 17, 18, and 20) via nuclear magnetic resonance spectroscopy NMR (Bruker LF110 BCA-Analyzer, Billerica, Mass.).

Hormones, Metabolites, and Hepatic Glycogen

Serum hormones and metabolites, as well as hepatic glycogen content were measured following a 4-5 hour fast, at ≈13:30. For glucagon, leptin, and β-hydroxybutyrate (BHB) analysis, trunk blood was collected into a 1.7 mL micro-centrifuge tubed and allowed to clot at room temperature for 15 minutes. Samples were then centrifuged at 4,400 rpm at 4° C. for 10 minutes. Serum was collected and stored at −80° C. until assay. For ghrelin analysis, approximately 100 μL of trunk blood was collected into a 1.7 mL micro-centrifuge tube containing 10 pt 0.5M EDTA (prepared in-house) and 2 μL of the enzyme inhibitor MAFP (Cayman Chemical Ann Arbor, Mich.). Samples were centrifuged at 4,400 rpm at 4° C. for 15 minutes. Plasma was collected and stored at −80° C. until analysis. Serum glucagon concentration was determined by ELISA (10-1281-01, Mercodia, Winston Salem, N.C.). Serum BHB concentration was determined by colorimetric assay (700190, Cayman Chemical, Ann Arbor, Mich.). Plasma ghrelin and serum leptin concentration was determined by ELISA (EZGRA-90K and EZML-82K, MilliporeSigma St. Louis, Mo.). Hepatic glycogen concentration was determined by colorimetric assay (Ab169558, Abcam, Cambridge, Mass.). Blood glucose measurements were made by hand held glucometer via tail pick immediately prior to euthanasia.

Hypoglycemic ITT

Following a 4-5 hour fast, mice received an intraperitoneal (IP) injection of insulin, (Humulin R, Lilly USA Indianapolis, Ind.) at a variable dose in order to induce hypoglycemia (blood glucose: 40-60 mg/dL) for 30 minutes. Blood glucose levels were determined via tail pick using a hand-held glucometer at −15, 0, 10, 20, 30, 45, and 60 minutes relative to insulin administration. At the 60-minute time point all animals were euthanized by CO2 inhalation followed by rapid decapitation. Trunk blood was collected for serum glucagon measurements as described above.

Results

Physiological Response to Severe Caloric Restriction

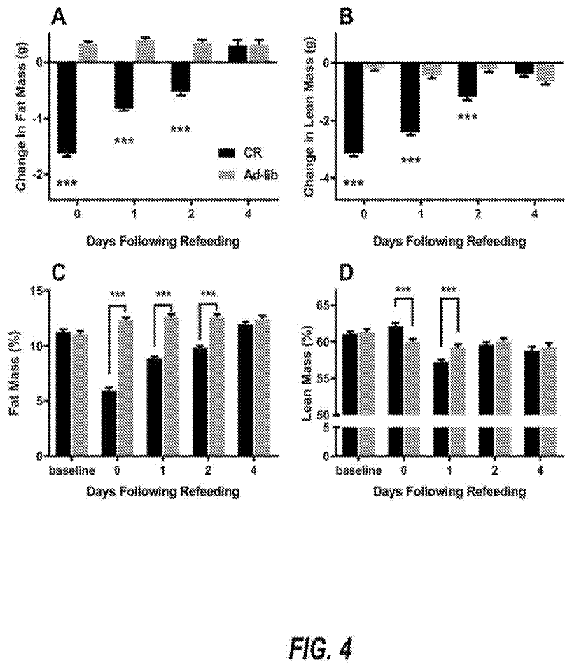

CR and Ad-lib mice had nearly identical body weights during the 10-day acclimation and baseline food intake portion of the experiment, with daily means ranging between 24.7-25.4 g and 24.7-25.2 g, respectively (FIG. 1, top panel). Within one day of exposure to the severe caloric restriction, CR mice had significantly lower mean body weight compared to Ad-lib mice, 22.9+0.2 g and 25.3+0.3 g, respectively [(p<0.0001); (FIG. 1, top panel). The mean body weights of CR mice continued to diverge from Ad-lib mice as the CR paradigm continued, reaching a maximum difference on the final day, 18.9+0.2 g and 24.9+0.2 g respectively (p<0.0001). CR and Ad-lib mice also had nearly identical body composition at baseline with mean percent fat mass of 11.3+0.2% and 11.1+0.2% respectively, and mean percent lean mass of 61.1+0.3% and 61.4+0.35% respectively (FIGS. 2C and 2D). On the final day of the paradigm CR mice displayed a mean reduction in fat mass of 1.63+0.06 g and a mean reduction in lean mass 3.1+0.1 g relative to baseline values, which was significantly different from Ad-lib mice [(p<0.0001); (FIG. 4, pane; A and B)]. This represented an approximately 50% drop in mean percent fat mass, from 11.3+0.2% to 5.9+0.3% (FIG. 4, panel C, Day 0). Due to this dramatic reduction in body fat, CR mice displayed an increase in mean percent lean mass on the final day of the CR paradigm which was significantly higher than that of Ad-lib mice, 62.2+0.4 versus 60.0+0.4%, respectively [(p<0.0001); (FIG. 4, pancel D, Day 0)].

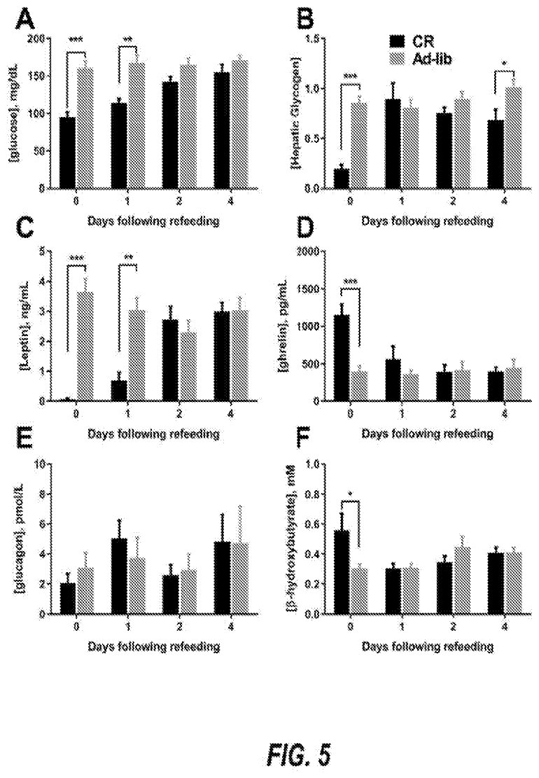

Mean free-living blood glucose values showed a similar pattern to body weight during the acclimation and baseline periods of the experiment, with CR and Ad-lib mice having similar mean glucose values ranging from 149.5-160.7 mg/dL and 150.9-158.7 mg/dL respectively (FIG. 1, middle panel). Within two days of exposure to the severe caloric restriction, CR mice displayed lower mean free-living glucose compared to Ad-lib mice, 117.7+2.6 dg/dL versus 156.5+3.1 mg/dL, respectively (p<0.0001). Mean free-living glucose of CR mice continued to diverge relative to Ad-lib mice for the remainder of the CR paradigm, reaching a nadir on the final day, 83+3.1 mg/dL versus 156.8+2.5 mg/dL respectively (p<0.0001). Mean fasting blood glucose was also reduced in CR mice relative to Ad-lib mice on the final day of the CR paradigm, 95.1+7.2 mg/dL versus 159.9+9.4 mg/dL, respectively [(p<0.0001); (FIG. 5, panel A)]. Fasted hepatic glycogen concentrations and serum leptin concentrations were also significantly reduced in CR mice relative to Ad-lib mice on the final day of the CR paradigm, 0.20+0.04 versus 0.86+0.07 (p <0.0001) and 0.07+0.03 ng/mL versus 3.6+0.4 ng/mL (p<0.0001), respectively (FIGS. 3B and 3C, Day 0). Mean serum ghrelin and BHB levels were significantly higher in CR mice relative to Ad-lib mice on the final day of the CR paradigm, 1153.0+145.1 pg/mL versus 399.4+70.8 pg/mL (p<0.0001) and 0.6+0.1 mM versus 0.30+0.03 mM (p=0.0166), respectively (FIGS. 3D and 3F, Day 0), while mean fasting glucagon levels were not significantly different (FIG. 3E). CR and Ad-lib mice had nearly identical food intake during the baseline food intake period of the experiment, with daily means ranging between 4.4-3.8 g and 4.5-3.9 g, respectively (FIG. 1, bottom panel).

Physiological Responses to Refeeding Following Exposure to Severe Caloric Restriction

Following the sixth day of caloric restriction, CR mice were given free access to food for up to four days (refeeding period). Mean body weight of CR mice remained significantly lower than Ad-lib mice during the first two days of the refeeding period (p<0.0001 and p=0.0366, respectively), before normalizing on the third and fourth day of refeeding (FIG. 1, top panel). Relative to Ad-lib mice, CR mice had significant reductions in mean change in fat mass relative to baseline and reduced percent fat mass on the first two days of refeeding (p<0.0001 for all 4 comparisons). These parameters normalized on the fourth day of refeeding (FIG. 4, panels A and C). Similarly, CR mice had significant reductions in mean change in lean mass relative to baseline on the first two days of refeeding (p<0.0001) before normalizing on the fourth day of refeeding (FIG. 4, panel B). The percent lean mass of CR mice was also significantly below that of Ad-lib mice on the first day of refeeding (p=0.0001), before normalizing on the second and fourth days (FIG. 4, panel D)). This restoration of body weight and body composition was accompanied by a significant increase in mean daily food intake in CR mice relative to Ad-lib mice on each of the four days of the refeeding period [(p<0.0001 for all 4 comparisons); (FIG. 1, bottom panel)].

Mean free-living blood glucose levels and fasting blood glucose levels of CR mice were both significantly lower than Ad-lib mice on the first day of the refeeding period (both p<0.0001). Although mean free-living and fasting blood glucose levels of CR mice remained numerically lower than Ad-lib mice for the remainder of the refeeding period, none of these comparisons reached statistical significance (FIG. 1, middle panel; FIG. 5, panel A). Mean fasting serum leptin levels continued to be significantly lower in CR mice relative to Ad-lib mice on the first day of refeeding (p=0.0001), but were not significantly different on the remaining days of refeeding. Mean fasting hepatic glycogen concentrations were similar between CR and Ad-lib mice during the refeeding period, although CR mice did have significantly lower levels on the fourth day of the refeeding period [(p=0.0443); (FIG. 5, panel B)]. Mean fasting serum ghrelin, glucagon, and BHB concentrations were not significantly different between CR and Ad-lib mice during any of the days of the refeeding period (FIG. 5, panels D, E, and F).

Hypoglycemic Counter-Regulation Following Exposure to Six Days of 60% Caloric Restriction

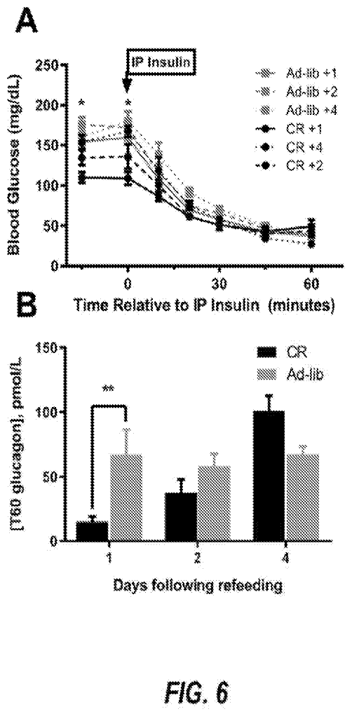

Hypoglycemic counter-regulation was assessed via a hypoglycemic ITT in six groups of mice: one group of CR mice and one group of Ad-lib mice on the first, second, and fourth day of the refeeding period. Due to differences in baseline blood glucose levels and insulin sensitivity between groups, a variable dose of insulin was administered to achieve similar levels of hypoglycemia during the ITT procedure. Mean blood glucose at 20, 30, 45, or 60 minutes post insulin injection was not significantly difference between any of the six groups (FIG. 6, panel A). The mean blood glucoses across all six groups at 30, 45, and 60 minutes post insulin injection was 58.0+3.3 mg/dL, 42.7+2.1 mg/dL, and 39.0+2.9 mg/dL, respectively. Trunk blood was collected 60 minutes post insulin injection and glucagon levels were measured. On the first day of the refeeding period CR mice had significantly reduced hypoglycemia-induced glucagon release compared to Ad-lib mice, 15.0+4.1 pmol/L versus 66.8+19.6 pmol/L respectively (p=0.0087). There was no significant differences in hypoglycemia-induced glucagon release on the second and fourth day of the refeeding period (FIG. 6, panel B).

Discussion

Findings

There is increasing evidence linking exposure to starvation with alterations in glucose homeostasis (Adamson et al., 1989; Mani et al., 2016; Mani & Zigman, 2017; Perry et al., 2018). In this study we extended these results to demonstrate that mice exposed to starvation, via six days of 60% CR, exhibit deficits in their counter-regulatory response to insulin-induced hypoglycemia. More importantly, this deficit persists beyond the cessation of CR and despite the normalization of both hyperglucagonemia and hepatic glycogen content. Our data also adds to the body of knowledge regarding the physiological response to refeeding following exposure to starvation. The time course of the restoration of hypoglycemic counter-regulation following refeeding was similar to that of the restoration of serum leptin levels, body composition, and fasting glucose levels to pre-starvation levels. In contrast, restoration of serum ketone and ghrelin levels to pre-starvation levels preceded the restoration of hypoglycemic counter-regulation. These findings demonstrate that 60% CR is a useful model to explore the mechanisms underlying the development of HAAF.

Relevance to Prior Studies

Severe caloric restriction paradigms have been routinely used to investigate the physiological response to starvation in both mice (Sun et al., 2008; Zhao et al., 2010; Li et al., 2012; Mani et al., 2016) and rats (Bois-Joyeux et al., 1990; Perry et al., 2018) via 50-60% CR or a 48-hour fast, respectively. The metabolic parameters measured on the final day of our severe caloric restriction are consistent with these prior studies. We observed an 4 fold increase in plasma ghrelin, a 4 fold decrease in plasma leptin, a 2 fold increase in BHB, a 75% reduction in hepatic glycogen content, and a 40-45% reduction in blood glucose levels (FIG. 1 and FIG. 5). Our CR paradigm produced a 24% reduction in body weight, with a sparing of % lean mass relative to % fat mass (FIG. 1 and FIG. 4). These data are also consistent with prior experiments utilizing severe CR in mice (Sun et al., 2008; Zhao et al., 2010; Li et al., 2012; Mani et al., 2016). Our failure to detect hyperglucagonemia on the final day of our CR (FIG. 5, panel E) is somewhat surprising, although not unprecedented given the mixed findings in the aforementioned studies. Although a 2 fold increase in glucagon is reported following exposure to severe CR in some studies (Zhao et al., 2010; Li et al., 2012; Perry et al., 2018), Mani et al (2016) reported glucagon levels following six days of CR which were 75% lower that glucagon levels following a 24-hour fast, and Bois-Joyeux et al. (1990) reported no difference in glucagon levels in 48-hour fasted rats relative to controls. The discrepancy could also be due to our low sample number at that time point. It should be noted that the glucagon assay used in the current study has been shown to have high accuracy relative to other commercial ELISAs and comparable accuracy with RIA assays (Wewer Albrechtsen et al., 2014).

Little data has been reported on the restoration of pre-starvation physiology during refeeding in rodent models of severe CR, thus our study makes significant contributions to the body of knowledge regarding the reversal of the physiological response to starvation following severe CR. We determined the time course of the reversal of the above metabolic and body composition parameters relative to ad libitum feed controls. Several of the key physiological responses to starvation were reversed to control levels within 24 hours. These included elevations in ghrelin and BHB, as well as the reduction in hepatic glycogen content. These results are consistent with the known biological regulation of circulating ghrelin (Sun et al., 2008; Mani & Zigman, 2017) and ketone levels (Owen, 2005; Watford, 2015; Rojas-Morales et al., 2016) and previous measurements of hepatic glycogen following refeeding in 48-hour fasted rats (Bois-Joyeux et al., 1990).

In contrast, leptin levels remained lower than controls on the first day of refeeding and this coincided with alterations in glucose homeostasis including reductions in free-living glucose (FIG. 1) and fasting glucose levels (FIG. 5, panel A) as well as a vastly reduced counter-regulatory response to insulin-induced hypoglycemia (FIG. 6, panel B). Leptin is a known regulator of energy homeostasis and neuroendocrine function (Kelesidis et al., 2010) and more recently has gained recognition as a potent regulator of glucose homeostasis (Meek & Morton, 2016). Although much of this work has focused on the glucose lowering effect of leptin (Chinookoswong et al., 1999; Yu et al., 2008; Perry et al., 2014), leptin has been shown to influence counter-regulation in response to both insulin- and fasting-induced hypoglycemia (Reno et al., 2015; Perry et al., 2018). Furthermore, circulating leptin levels fall to undetectable levels following induction of HAAF-like symptoms in rats via recurrent exposure to insulin-induced hypoglycemia (Reno et al., 2015). Altogether, these results indicate that alterations in leptin levels may play a role in the development of impaired hypoglycemic counter-regulation.

Clinical Implications

Without wishing to be bound by theory, our results indicate that the physiological response to starvation can provide new insights into the physiologic underpinnings of HAAF. HAAF is highly prevalent in diabetic patients, dangerous, and difficult to manage clinically. Patients with longstanding diabetes are vulnerable to exposure to recurrent, treatment-induced hypoglycemic episodes, which lead to the development of HAAF and further risk of hypoglycemic complications. It is estimated that 40% of diabetic patients are at high risk for hypoglycemic complications (Hopkins et al., 2012), and that 20% have HAAF (Geddes et al., 2008). Severe hypoglycemia is associated with a 3.4-fold increase risk of death (McCoy et al., 2012), and long-term longitudinal studies indicate that 6-10% of individuals with type 1 diabetes die as a result of acute hypoglycemia (Skrivarhaug et al., 2006; Jacobson et al., 2007; Feltbower et al., 2008). Undoubtedly, a large majority of these deaths are influenced by the development of HAAF (Cryer, 2012). Although HAAF can often be reversed by prolonged avoidance of hypoglycemia, intensive glycemic therapies aimed at minimizing hyperglycemia are directly linked to unanticipated increases in hypoglycemic episodes (Control & Group, 1997; Gerstein et al., 2008; Cryer, 2011; Finfer et al., 2012). The need for a better understanding of the mechanisms underlying the development of HAAF is only increasing as the incidence of glucose lowering therapies in diabetes treatment increases.

Various mechanisms of underlying the development of HAAF have been proposed, including altered CNS glucose utilization, modified CNS glycogen storage, and alterations in neurotransmission and/or neuromodulation, yet the data supporting each of these mechanisms are largely inconsistent (Beall et al., 2012; Cryer, 2013b; Lontchi Yimagou et al., 2018). The results of the present study demonstrate that HAAF is caused by an adaptive alteration glucose homeostasis induced by exposure to antecedent conditions which are normally present during starvation. This could explain the long established, but paradoxical, observation that HAAF is simultaneously maladaptive and adaptive (reviewed in Cryer, 2012). For example, although HAAF leads to progressive loss of the CNS response to hypoglycemia, it is also associated with neuroprotective effects (Puente et al., 2010; Cryer, 2012; Litvin et al., 2013). Similar neuroprotective effects are induced by caloric restriction and ketogenic diets (e.g. Yudkoff et al., 2007; Maalouf et al., 2009; Yuen & Sander, 2014), which may indicate a common etiology underlying the two phenomena. The data indicate a temporal association between the development of HAAF-like symptoms and the presence of hypoleptinemia, hyperghrelinemia, or ketosis. Further studies will be needed to determine if any of these conditions in isolation are sufficient to produce deficits in hypoglycemic counter-regulation. Positive results in these investigations have the potential to drive new therapeutic and treatment strategies for detecting and reversing HAAF in patients with diabetes.

### REFERENCES CITED IN THIS EXAMPLE

Adamson U, Lins P E & Grill V (1989). Fasting for 72 h decreases the responses of counterregulatory hormones to insulin-induced hypoglycaemia in normal man. Scandinavian journal of clinical and laboratory investigation 49, 751-756.
Beall C, Ashford M L & McCrimmon R J (2012). The physiology and pathophysiology of the neural control of the counterregulatory response. American Journal of Physiology-Regulatory Integrative and Comparative Physiology 302, R215-R223.
Bluml S, Shic F, Lai C L, Yahya K, Lin A & Ross B (2002). Glutamate—glutamine cycling in epileptic patients on ketogenic diets. In Proceedings, 10th International Society of Magnetic Resonance in Medicine 1, 417.
Bois-Joyeux B, Chanez M & Peret J (1990). Age-dependent glycolysis and gluconeogenesis enzyme activities in starved-refed rats. Diabete & metabolisme 16, 504-512.
Chinookoswong N, Wang J L & Shi Z Q (1999). Leptin restores euglycemia and normalizes glucose turnover in insulin-deficient diabetes in the rat. Diabetes 48, 1487-1492.
Control TD & Group CTR (1997). Hypoglycemia in the Diabetes Control and Complications Trial. Diabetes 46, 271-286.
Cryer P E (2005). Mechanisms of hypoglycemia-associated autonomic failure and its component syndromes in diabetes. Diabetes 54, 3592-3601.
Cryer P E (2011). Death during Intensive Glycemic Therapy of Diabetes: Mechanisms and Implications. American Journal of Medicine 124, 993-996.
Cryer P E (2012). Severe Hypoglycemia Predicts Mortality in Diabetes. Diabetes care 35, 1814-1816.
Cryer P E (2013a). Mechanisms of Hypoglycemia-Associated Autonomic Failure in Diabetes. New England Journal of Medicine 369, 362-372.
Cryer P E (2013b). Mechanisms of Hypoglycemia-Associated Autonomic Failure in Diabetes. New England Journal of Medicine 369, 362-372.
Feltbower R G, Bodansky H J, Patterson C C, Parslow R C, Stephenson C R, Reynolds C & McKinney P A (2008). Acute complications and drug misuse are important causes of death for children and young adults with type 1 diabetes: results from the Yorkshire Register of diabetes in children and young adults. Diabetes care 31, 922-926.
Finfer S, Liu B, Chittock D R, Norton R, Myburgh J A, McArthur C, Mitchell I, Foster D, Dhingra V, Henderson W R, Ronco J J, Bellomo R, Cook D, McDonald E, Dodek P, Hebert P C, Heyland D K, Robinson B G & Investigators N-SS (2012). Hypoglycemia and Risk of Death in Critically Ill Patients. New England Journal of Medicine 367, 1108-1118.
Geddes J, Schopman J E, Zammitt N N & Frier B M (2008). Prevalence of impaired awareness of hypoglycaemia in adults with Type 1 diabetes. Diabetic medicine: a journal of the British Diabetic Association 25, 501-504.
Gerstein H C, Miller M E, Byington R P, Goff D C, Jr., Bigger J T, Buse J B, Cushman W C, Genuth S, Ismail-Beigi F, Grimm R H, Jr., Probstfield J L, Simons-Morton D G, Friedewald W T & Act Control Cardiovasc Risk D (2008). Effects of intensive glucose lowering in type 2 diabetes. New England Journal of Medicine 358, 2545-2559.
Goldstein J L, Zhao T J, Li R L, Sherbet D P, Liang G & Brown M S (2011). Surviving starvation: essential role of the ghrelin-growth hormone axis. Cold Spring Harbor symposia on quantitative biology 76, 121-127.
Gulanski B I, De Feyter H M, Page K A, Belfort-DeAguiar R, Mason G F, Rothman D L & Sherwin R S (2013). Increased Brain Transport and Metabolism of Acetate in Hypoglycemia Unawareness. Journal of Clinical Endocrinology & Metabolism 98, 3811-3820.
Hojlund K, Wildner-Christensen M, Eshoj O, Skjaerbaek C, Holst J J, Koldkjaer O, Moller Jensen D & Beck-Nielsen H (2001). Reference intervals for glucose, beta-cell polypeptides, and counterregulatory factors during prolonged fasting. American Journal of Physiology—Endocrinology and Metabolism 280, E50-E58.
Hopkins D, Lawrence I, Mansell P, Thompson G, Amiel S, Campbell M & Heller S (2012). Improved biomedical and psychological outcomes 1 year after structured education in flexible insulin therapy for people with type 1 diabetes: the U.K. DAFNE experience. Diabetes care 35, 1638-1642.
Jacobson A M, Musen G, Ryan C M, Silvers N, Cleary P, Waberski B, Burwood A, Weinger K, Bayless M, Dahms W & Harth J (2007). Long-term effect of diabetes and its treatment on cognitive function. The New England journal of medicine 356, 1842-1852.
Kelesidis T, Kelesidis I, Chou S & Mantzoros C S (2010). Narrative Review: The Role of Leptin in Human Physiology: Emerging Clinical Applications. Annals of Internal Medicine 152, 93-+.
Khunti K, Davies M, Majeed A, Thorsted B L, Wolden M L & Paul S K (2014). Hypoglycemia and Risk of Cardiovascular Disease and All-Cause Mortality in Insulin-Treated People With Type 1 and Type 2 Diabetes: A Cohort Study. Diabetes care.
Li R L, Sherbet D P, Elsbernd B L, Goldstein J L, Brown M S & Zhao T J (2012). Profound hypoglycemia in starved, ghrelin-deficient mice is caused by decreased gluconeogenesis and reversed by lactate or fatty acids. The Journal of biological chemistry 287, 17942-17950.
Litvin M, Clark A L & Fisher S J (2013). Recurrent hypoglycemia: boosting the brain's metabolic flexibility. J Clin Invest 123, 1922-1924.
Lontchi-Yimagou E, You J Y, Carey M, Gabriely I, Shamoon H & Hawkins M (2018). Potential approaches to prevent hypoglycemia-associated autonomic failure. Journal of Investigative Medicine 66, 641-647.
Maalouf M, Rho J M & Mattson M P (2009). The neuroprotective properties of calorie restriction, the ketogenic diet, and ketone bodies. Brain Research Reviews 59, 293-315.
Mani B K, Osborne-Lawrence S, Vijayaraghavan P, Hepler C & Zigman J M (2016). betal-Adrenergic receptor deficiency in ghrelin-expressing cells causes hypoglycemia in susceptible individuals. The Journal of Clinical Investigation 126, 3467-3478.
Mani B K & Zigman J M (2017). Ghrelin as a Survival Hormone. Trends in endocrinology and metabolism: TEM 28, 843-854.
McCoy R G, Van Houten H K, Ziegenfuss J Y, Shah N D, Wermers R A & Smith S A (2012). Increased Mortality of Patients With Diabetes Reporting Severe Hypoglycemia. Diabetes care 35, 1897-1901.
McDougal D H, Darpolor M M, DuVall M A, Sutton E F, Morrison C D, Gadde K M, Redman L M & Carmichael O T (2018). Glial acetate metabolism is increased following a 72-h fast in metabolically healthy men and correlates with susceptibility to hypoglycemia. Acta diabetologica.
Meek T H & Morton G J (2016). The role of leptin in diabetes: metabolic effects. Diabetologia 59, 928-932.
Melo T M, Nehlig A & Sonnewald U (2006). Neuronal—glial interactions in rats fed a ketogenic diet. Neurochemistry International 48, 498-507.
Moheet A, Kumar A, Eberly L E, Kim J, Roberts R & Seaquist E R (2014). Hypoglycemia-Associated Autonomic Failure in Healthy Humans: Comparison of Two vs Three Periods of Hypoglycemia on Hypoglycemia-Induced Counterregulatory and Symptom Response 5 Days Later. Journal of Clinical Endocrinology & Metabolism 99, 664-670.
Owen O E (2005). Ketone bodies as a fuel for the brain during starvation. Biochemistry and Molecular Biology Education 33, 246-251.
Perry R J, Wang Y, Cline G W, Rabin-Court A, Song J D, Dufour S, Zhang X M, Petersen K F & Shulman G I (2018). Leptin Mediates a Glucose-Fatty Acid Cycle to Maintain Glucose Homeostasis in Starvation. Cell 172, 234-248 e217.
Perry R J, Zhang X M, Zhang D, Kumashiro N, Camporez J P, Cline G W, Rothman D L & Shulman G I (2014). Leptin reverses diabetes by suppression of the hypothalamic-pituitary-adrenal axis. Nature medicine 20, 759-763.
Puente E C, Silverstein J, Bree A J, Musikantow D R, Wozniak D F, Maloney S, Daphna-Iken D & Fisher S J (2010). Recurrent moderate hypoglycemia ameliorates brain damage and cognitive dysfunction induced by severe hypoglycemia. Diabetes 59, 1055-1062.
Reno C M, Ding Y & Sherwin R (2015). Leptin acts in the brain to influence hypoglycemic counterregulation: disparate effects of acute and recurrent hypoglycemia on glucagon release. American journal of physiology. Endocrinology and metabolism 309, E960-967.
Reno C M, Litvin M, Clark A L & Fisher S J (2013). Defective counterregulation and hypoglycemia unawareness in diabetes: mechanisms and emerging treatments. Endocrinology and Metabolism Clinics of North America 42, 15-38.
Rogers R C, McDougal D H & Hermann G E (2017). Hindbrain Astrocyte Glucodetectors and Counterregulation. In Appetite and Food Intake: Central Control. ed. Harris R B S, pp. 205-228. CRC Press/Taylor & Francis, Boca Raton (Fla.).
Rojas-Morales P, Tapia E & Pedraza-Chaverri J (2016). beta-Hydroxybutyrate: A signaling metabolite in starvation response? Cellular signalling 28, 917-923.
Senthilkumaran M, Zhou X F & Bobrovskaya L (2016). Challenges in Modelling Hypoglycaemia-Associated Autonomic Failure: A Review of Human and Animal Studies. International journal of endocrinology 2016, 9801640.
Skrivarhaug T, Bangstad H J, Stene L C, Sandvik L, Hanssen K F & Joner G (2006). Long-term mortality in a nationwide cohort of childhood-onset type 1 diabetic patients in Norway. Diabetologia 49, 298-305.
Soeters M R, Soeters P B, Schooneman M G, Houten S M & Romijn J A (2012). Adaptive reciprocity of lipid and glucose metabolism in human short-term starvation. American Journal of Physiology—Endocrinology and Metabolism 303, E1397-E1407.
Sun Y, Butte N F, Garcia J M & Smith R G (2008). Characterization of adult ghrelin and ghrelin receptor knockout mice under positive and negative energy balance. Endocrinology 149, 843-850.
Taborsky G J & Mundinger T O (2012). Minireview: The Role of the Autonomic Nervous System in Mediating the Glucagon Response to Hypoglycemia. Endocrinology 153, 1055-1062.
Watford M (2015). Starvation: Metabolic Changes. In eLS. John Wiley & Sons, Ltd.
Wewer Albrechtsen N J, Hartmann B, Veedfald S, Windelov J A, Plamboeck A, Bojsen-Moller K N, Idorn T, Feldt-Rasmussen B, Knop F K, Vilsboll T, Madsbad S, Deacon C F & Holst J J (2014). Hyperglucagonaemia analysed by glucagon sandwich ELISA: nonspecific interference or truly elevated levels? Diabetologia 57, 1919-1926.
Winnick J J, Kraft G, Gregory J M, Edgerton D S, Williams P, Hajizadeh I A, Kamal M Z, Smith M, Farmer B, Scott M, Neal D, Donahue E P, Allen E & Cherrington A D (2016). Hepatic glycogen can regulate hypoglycemic counterregulation via a liver-brain axis. The Journal of Clinical Investigation 126, 2236-2248.
Yu X, Park B H, Wang M Y, Wang Z V & Unger R H (2008). Making insulin-deficient type 1 diabetic rodents thrive without insulin. Proceedings of the National Academy of Sciences of the United States of America 105, 14070-14075.
Yudkoff M, Daikhin Y, Melo T M, Nissim I & Sonnewald U (2007). The ketogenic diet and brain metabolism of amino acids: Relationship to the anticonvulsant effect. In Annual review of nutrition, vol. 27, pp. 415-430.
Yuen A W C & Sander J W (2014). Rationale for using intermittent calorie restriction as a dietary treatment for drug resistant epilepsy. Epilepsy & Behavior 33, 110-114.
Zhao T J, Liang G, Li R L, Xie X, Sleeman M W, Murphy A J, Valenzuela D M, Yancopoulos G D, Goldstein J L & Brown M S (2010). Ghrelin O-acyltransferase (GOAT) is essential for growth hormone-mediated survival of calorie-restricted mice. Proceedings of the National Academy of Sciences of the United States of America 107, 7467-7472.

### Example 3

A. Specific Aims:

Patients with diabetes are vulnerable to hypoglycemia, and exposure to hypoglycemia is associated with an increased risk of all-cause mortality in these patients. Patients with longstanding diabetes also often experience recurrent hypoglycemic episodes due to poorly regulated hyperinsulinemia and inadequate glucagon responses. Recurrent bouts of hypoglycemia leads to the development of hypoglycemia associated autonomic failure (HAAF), which is characterized by critically reduced neuroendocrine responses to hypoglycemia as well as hypoglycemia unawareness. HAAF leaves persons with diabetes vulnerable to life-threatening episodes of severe hypoglycemia and is a significant barrier to diabetes treatment.

HAAF is thought to be caused by maladaptive changes in the central nervous system and/or the peripheral neuroendocrine system that are instigated by exposure to recurrent hypoglycemia. This project will validate that the development of HAAF is driven by hypoleptinemia. Prior work by the PI and others has generated three key observations that demonstrate a link between hypoleptinemia and the development of HAAF. These include: 1) Exposure to recurrent hypoglycemia in rats, a known antecedent of HAAF, causes hypoleptinemia; 2) Exposure to short-term starvation, a known antecedent of hypoleptinemia, causes HAAF-like symptoms in both mice and humans; 3) The induction of HAAF-like symptoms in mice via starvation can be prevented with leptin supplementation during starvation. Based on these observations, leptin supplementation can prevent the development of HAAF.

Securing extramural funding for a clinical study validating this is currently limited by the absence of two data sets. First, we must validate in an animal model if leptin supplementation during exposure to recurrent hypoglycemia can prevent the development of HAAF-like symptoms. Second, we must validate that exposure to recurrent hypoglycemia causes hypoleptinemia in humans, thus establishing a common link between our basic and clinical data. Therefore, the overarching goal of this project is to validate that hypoleptinemia drives the development of HAAF via the following specific aims:

Aim 1: Validate whether leptin supplementation can prevent the development of HAAF-like symptoms in rats.

We will expose leptin and vehicle treated animals to recurrent hypoglycemia to induce HAAF-like symptoms. Without wishing to be bound by theory, leptin treatment will prevent the development of HAAF-like symptoms.

Aim 2: Validate whether exposure to recurrent hypoglycemia decreases leptin levels in humans. We will measure leptin levels before, during, and two days following exposure to three bouts of insulin-induced hypoglycemia. Without wishing to be bound by theory, exposure to recurrent hypoglycemia will lead to a >50% reduction in leptin levels.

Aim 3: Validate whether leptin levels predict susceptibility to hypoglycemia. We will measure leptin levels just prior to exposure to insulin-induced hypoglycemia and determine how well this measurement predicts the magnitude of the neuroendocrine responses to the subsequent bout of hypoglycemia. Without wishing to be bound by theory, individuals with low leptin levels will be more susceptible to hypoglycemia, that is, as an individual's leptin levels decrease, their neuroendocrine response to hypoglycemia will also decrease.

Without wishing to be bound by theory, these studies will demonstrate that hypoleptinemia functionally drives the development of HAAF. These results will not only identify the first endocrine mechanism causal to HAAF, but they will also identify a clinically tractable treatment for the prevention or treatment of HAAF.

B. Research Plan:

B1. Significance: HAAF is highly prevalent, difficult to manage clinically, and leads to life-threatening episodes of severe hypoglycemia (1). HAAF is a significant barrier to the maintenance of healthy glucose levels in both type 1 and 2 diabetes (2, 3). It is estimated that 40% of diabetic patients are at high risk for severe hypoglycemia (4), and that 20% have HAAF (5). Severe hypoglycemia is associated with a 3.4 fold increased risk of death (6) and longitudinal studies indicate that 6-10% of individuals with type 1 diabetes die as a result of acute hypoglycemia (7-9). There is no treatment for HAAF other than stringent avoidance of hypoglycemia, which is extremely difficult to accomplish clinically in high-risk patients (10). This project will validate leptin as a composition for the treatment or prevention of HAAF. Thus, positive results would be of high clinical significance.

B2. Innovation: The innovation of this project is clearly demonstrated in three key areas: First, our focus on hypoleptinemia as an antecedent of HAAF is new. Second, our use of the hyperinsulinemic-hypoglycemic clamp procedure in humans (Aims 2 and 3) is at the forefront of neuroendocrine research. Pennington Biomedical is one of the few institutions currently utilizing this procedure to study hypoglycemic counter-regulation in human subjects. Lastly, the scientific premise of this research proposal is unique and conceptually innovative. The studies indicate that increased risk for severe hypoglycemia results from a normal physiological response to starvation that is induced by recurrent iatrogenic hypoglycemia.

HAAF as a physiological response to fasting—Although HAAF is commonly experienced by diabetic patients, diabetes is not required for its development. HAAF-like symptoms can be induced in metabolically heathy humans and rodents via recurrent exposure to insulin-induced hypoglycemia (11, 12). Thus, HAAF represents a pathophysiological adaptation brought about by repetitive exposure to hypoglycemia irrespective of disease state. Furthermore, the known antecedent of HAAF, insulin-induced hypoglycemia, is a relatively modern phenomenon which has only arisen following the adoption of exogenous insulin therapy. From an evolutionary perspective, this form of hypoglycemia would be exceedingly rare and unlikely to drive such distinct and ubiquitous physiological adaptations (13). In fact, glucose homeostasis is so well controlled in mammals that hypoglycemia is rarely experienced except during prolonged starvation (14, 15). Thus, HAAF is caused by an adaptive alteration in glucose homeostasis induced by exposure to antecedent conditions that are normally present during starvation. In support of this, the PI has shown that a 72-hour fast in humans induces changes in cerebral metabolism (16) that are strongly associated with the development of HAAF (17). In addition, Adamson et al. (18) demonstrated a significant reduction in the neuroendocrine response to insulin-induced hypoglycemia directly following a 72-hour fast in humans. We have also tested this in the mouse model by utilizing six days of 60% caloric restriction (CR) to induce a starvation response. One day following refeeding, CR mice displayed abnormal hypoglycemic counter-regulation, indicated by significantly reduced hypoglycemia-induced glucagon release, relative to ad libitum (Ad-lib) fed control mice not exposed to CR [(FIGS. 1A and 1B); (19)]. This phenomenon was preceded by a drastic reduction in leptin levels in CR mice, which began three days prior to refeeding and remained through the first day of refeeding [(see Day −3, 0, and 1 in FIG. 1C)]. More significantly, the CR-induced loss of counter-regulation was completely reversed by preventing the drop-in leptin levels via twice-daily exogenous leptin treatment (FIGS. 1D and 1E). All together, these results indicate that HAAF may be driven by physiological adaptations triggered by hypoleptinemia, which can be prevented with leptin supplementation.

B.3. Research Approach:

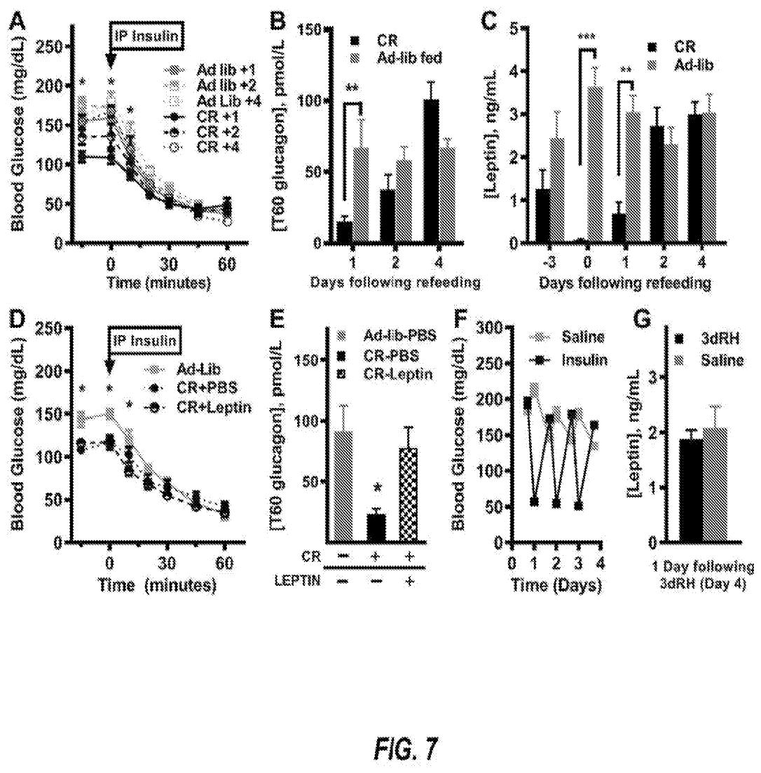

Project overview—We will use a combination of basic and clinical science approaches to validate that decreased leptin levels, induced by exposure to recurrent hypoglycemia, leads to impaired hypoglycemic counter-regulation. Aim 1 (basic) will focus on demonstrating a leptin mediated rescue of counter-regulation following CR (FIG. 7, panel E). We will utilize a similar paradigm to validate if leptin mediated rescue of counter-regulation following recurrent hypoglycemia. Aims 2 and 3 (clinical) will validate whether exposure to recurrent hypoglycemia leads to reduced leptin concentrations in human subjects and whether low leptin levels are associated with impaired counter-regulation. Positive results would establish the following data: 1) exposure to hypoglycemia leads to hypoleptinemia in humans and 2) blocking hypoglycemia-induced hypoleptinemia can prevent HAAF.

Aim 1: Validate if leptin supplementation can prevent the development of HAAF-like symptoms in rats.

Rationale—Exposure to three days of recurrent hypoglycemia (3dRH) is commonly used to generate a rat model of HAAF (11). In addition to a loss in hypoglycemic counter-regulation, 3dRH also leads to hypoleptinemia in rats (20). Although acute leptin treatment does not reverse the effects of 3dRH (20), our data in CR mice indicates that prevention of the hypoleptinemic state is necessary to prevent the induction of HAAF. Thus, this experiment will validate whether leptin supplementation during the 3dRH paradigm rescues the loss of hypoglycemic counter-regulation in rats. It should be noted that in contrast to the rat model, it is almost universally accepted that 3dRH does not produce HAAF-like symptoms in mice (11). The studies have demonstrated that 3dRH also does not lead to hypoleptinemia in mice (FIG. 7, panels F and G), which is consistent with our principle that hypoleptinemia is required for the development of HAAF.

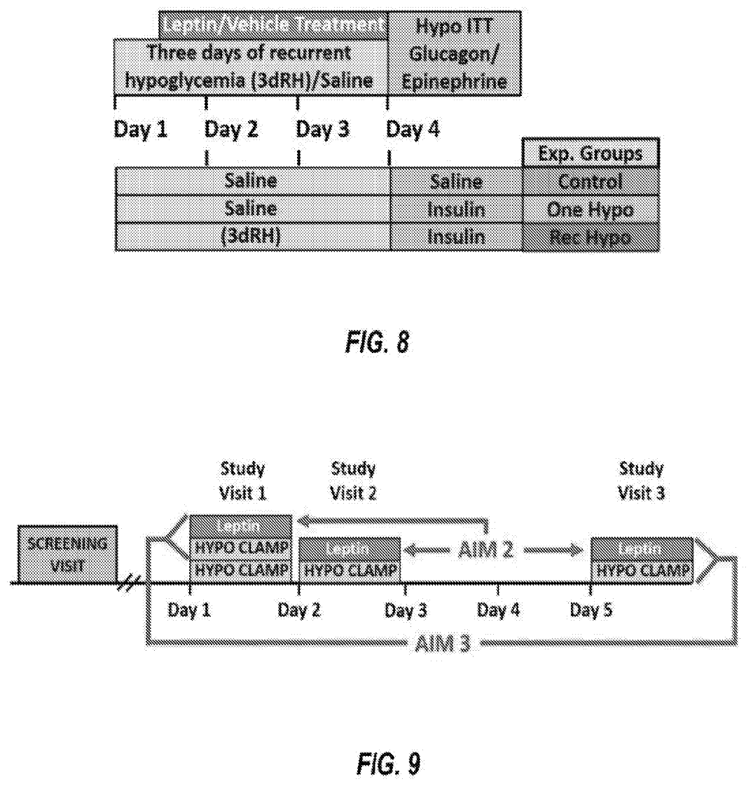

Design—Leptin or vehicle treated male and female rat will be exposed to 3dRH and the neuroendocrine response to hypoglycemia will be assessed the following day via hypoglycemic insulin tolerance test (see top of FIG. 8). Our 3dRH protocol is follows: on three consecutive days following a overnight fast, rats will either receive insulin (up to 10 U/kg IP) or saline, and blood glucose levels will be measured every 20 minutes for 140 minutes to confirm induction of hypoglycemia (e.g. FIG. 7, panel F). On the following day, hypoglycemia-induced increases in either glucagon or epinephrine will be assessed via a hypoglycemic insulin tolerance test (ITT). Our hypoglycemia ITT protocol is as follows: following an overnight fast, rats will either receive insulin or saline and then euthanized 60 minutes post-injection for collection of blood to determine levels of glucagon and epinephrine via ELISA (e.g. FIG. 7, panels B and E). Our Leptin treatment protocol is as follows: Rats will receive twice daily injections (at ˜8 am and ˜5 pm) of either recombinant rat leptin or vehicle. Treatment will begin at ˜5 pm of the first day of the 3dRH protocol and continue through the third day of the protocol. Leptin will be administered at 0.5 μg/g for the first two injections and then at 1 μg/g for the remaining three injections. This schedule and dosing is based on our prior CR experiments in mice (FIG. 7, panel E). A two-way ANOVA with two levels of treatment (leptin vs. vehicle) and three levels of hypoglycemic exposure [Control, One Hypo, and Rec Hypo (recurrent hypoglycemia); (see bottom of FIG. 2 for explanation of groups)] will be used to separately analyze the glucagon and epinephrine results. P-values <0.05 will be considered significance.

Results and Implications—Without wishing to be bound by theory, hypoglycemia induced levels of both glucagon and epinephrine will be significantly reduced in the vehicle treated Rec Hypo group relative to the One Hypo group. Without wishing to be bound by theory, leptin treatment will rescue this effect, i.e. the glucagon and epinephrine levels of leptin treated Rec Hypo rats will be similar to that of the leptin treated One Hypo rats. These results would have a significant impact on the field and provide impetus for pursuing leptin supplementation to prevent or treat HAAF in diabetic patients.

Potential problems and alternative strategies—We plan to initially confirm in a small cohort of rats that 3dRH produces significant reductions in the leptin levels and that our leptin dosing strategy is effective in reversing this effect. Based on known sex differences in hypoglycemic counter-regulation, a differential effect in male and female rats will be observed. We therefore plan to utilize a linear effects model to evaluate sex-based differences in our data.

Clinical study rationale (Aims 2 and 3)—In order to support further investigation into leptin as a treatment for HAAF, the effect of recurrent hypoglycemia on leptin levels in humans must be established. Although acute changes in leptin during exposure to hypoglycemia has been investigated with mixed results (See 22, 23 for a review of these studies), no study has investigated longer-term effects following acute or recurrent hypoglycemia.

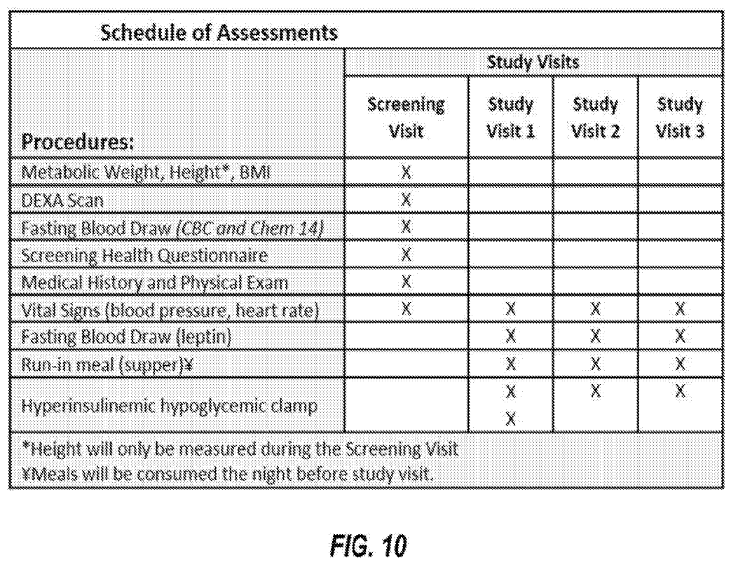

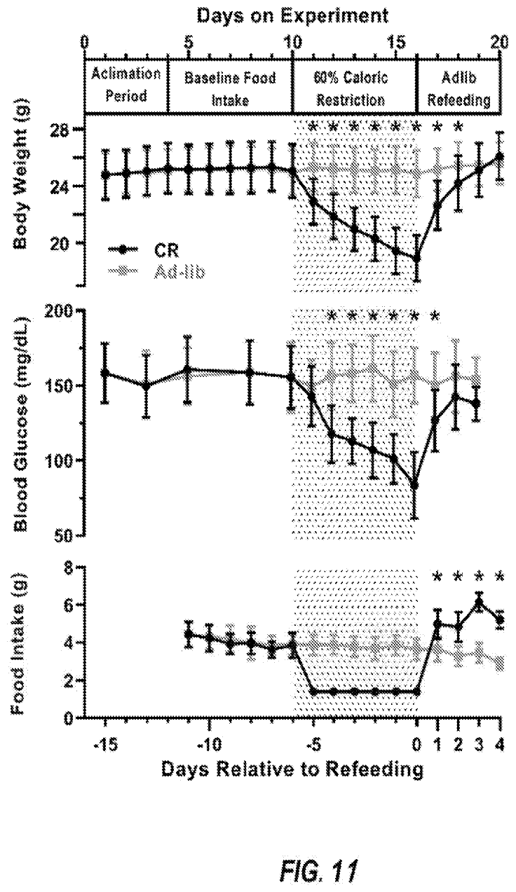

Clinical Study design—We will utilize a prospective observational study design to validate that exposure to a previously validated recurrent hypoglycemia paradigm (e.g.12) will decrease leptin levels in metabolically healthy men and women (see FIG. 3 for study design). We will measure each subject's fasting leptin levels three times: at baseline, one day later, and four days later (Aim 2). We also assess whether there is an association between leptin and the neuroendocrine response to hypoglycemia measured at baseline and following exposure to recurrent hypoglycemia (Aim 3). Subjects will complete a screening visit and three study visits. See FIG. 10 for a schedule of assessments. Primary endpoint: 50% decrease in fasting leptin levels. Secondary endpoints: Changes in hypoglycemia-induced levels of epinephrine, norepinephrine, cortisol, and glucagon.

Study Subjects—We will enroll up to 10 healthy individuals (goal n=8 completers; 4 men and 4 women). A subject's duration of participation will be approximately seven days. Inclusion criteria: Males and females from 18 to 40 years of age with a BMI between 20-27.9 kg/m2 and percent body fat by DEXA between 7-20% (males) and 10-30% (females), with a negative urine pregnancy test at screening and at Study Visit 1. Exclusion criteria: Fasting blood glucose >126 mg/dL, screening blood pressure >140/90 mmHg, use of medications affecting glucose metabolism, history of diabetes or cardiac disease, and liver insufficiency.

Subject recruitment and screening—Subjects will be recruited via IRB approved recruitment materials Screening: Eligible individuals will be scheduled for a screening visit at the PBRC outpatient clinic for final determination of eligibility. After providing written informed consent, the following procedures will be completed: anthropometrics, vital signs, DEXA, blood draw, Screening Heath Questionnaire, and medical history and physical examination. Subjects who satisfy the eligibility criteria will be invited to return for Study Visit 1.

Study Visits-Subjects are provided a run-in meal (supper) to be consumed the evening before all three study visits and each visit will require an overnight fast.
Study Visit 1: Blood is collected, and vital signs as well as anthropometrics measured. Following these, baseline neuroendocrine responses to hypoglycemia are measured via a hyperinsulinemic-hypoglycemic clamp procedure. Subjects will then consume a low carbohydrate breakfast (<10 g of carbohydrate) and blood glucose is maintained between 80-100 mg/dL via a variable intravenous infusion of 20% dextrose for two hours. Following this interval, a second hyperinsulinemic-hypoglycemic clamp is performed. After completion of the second clamp, subjects are fed lunch and discharged
Study Visit 2: Blood is collected, and vital signs as well as anthropometrics measured. Following these, a hyperinsulinemic-hypoglycemic clamp is performed. After completion of the clamp, participants are fed lunch and discharged from the Inpatient Clinic.
Study Visit 3: Blood is collected, and vital signs as well as anthropometrics measured. Following these, neuroendocrine responses to hypoglycemia are measured via a hyperinsulinemic-hypoglycemic clamp procedure. After completion of the clamp, participants are fed lunch and discharged from the Inpatient Clinic.

Clinical Procedures and Methods:

Anthropometrics: Fasting body weight is collected with subjects wearing a hospital gown and underwear.

Hyperinsulinemic-hypoglycemic clamp: After an overnight 10-hour fast, an intravenous catheter will be placed in an antecubital vein for infusion of insulin and glucose. A second catheter will be placed retrograde in a dorsal vein of the contra-lateral hand for blood withdrawal. The hand will be placed in a heating box or pad at 70° C. for arterialization of venous blood. Insulin infusion: A primed infusion of regular insulin (120 mU/min/m2) along with an infusion of potassium phosphate (4 mEq/h) will be initiated and continued for approximately 2 hours. Beginning 20 minutes prior to the start of the insulin infusion, arterialized plasma glucose will be measured at 5-minute intervals via finger stick. Following initiation of insulin infusion, blood glucose will be allowed to fall 50 mg/dL (+/−5 mg/dL) and then maintained at this level for 75 minutes using a variable infusion of 20% dextrose. Following discontinuation of the insulin and potassium infusion, blood glucose will be normalized at ˜100 mg/dl via dextrose infusion. Blood sampling protocol: In addition to the finger stick glucose measurements, blood will be collected every 15 minutes starting 15 minutes prior to insulin infusion for determination of glucose and insulin levels. Potassium levels will be measured at three of these time points: −20, 60, and 120 minutes (relative to the start of the insulin infusion). During the first clamp of Study Visit 1 and the Study Visit 3 clamp, additional blood will be collected for measurement of the counterregulatory hormones: glucagon, epinephrine, norepinephrine, and cortisol. These collections will occur every 15 minutes starting 15 minutes prior to initiation of the insulin infusion.

Vital Signs: Seated vital signs (blood pressure and heart rate) is measured after a 5 minute rest.

Safety Monitoring/Adverse Events—The study is considered greater than minimal risk due to the use of hyperinsulinemic clamp procedures in healthy subjects. The PBRC Inpatient Unit has one of highest levels of experience with hyperinsulinemic clamp procedures and has developed extensive precautions to prevent subjects' blood glucose from dropping to an unsafe level. All adverse events will be reviewed on an ongoing basis.

Statistical Analysis—A linear mixed effect model will be used to estimate partial correlations of leptin measurements over time. All subjects with baseline and at least one follow-up time will be used in the primary analysis (Aim 2). The secondary analysis will be based on completers only (aim 3). Power analysis: The estimates for our primary outcome, percent change in leptin levels, is based on prior studies. In this analysis an overall change from baseline of 50% in leptin levels with a standard deviation of 5.8% was used. With a total of 5 completers, this study would have at least 80% power to detect a 50% change in leptin levels. By enrolling 8 subjects, we ensure ample power while accounting for attrition, sex differences, and incomplete data.

Aim 2: Determine if exposure to recurrent bouts of insulin-induced hypoglycemia leads to decreased leptin levels. A mixed effect linear model will be used to model this outcome. Interactions of day will be used to test for both overall effect and linear trends over time.

Aim 3: Validate whether leptin levels are predictive of susceptibility to hypoglycemia. Linear mixed effect model will be used to estimate partial correlations between subjects' leptin levels and neuroendocrine response to hypoglycemia. More specifically, comparisons will be made between leptin and data from the 1st clamp during Study Visit 1, and Study Visit 3 leptin and clamp data, which represents measurements prior to and following recurrent hypoglycemia, respectively (see FIG. 9 for schedule of visits and their relation to the project aims). Steady-state epinephrine levels (the mean of the last three epinephrine values during the hypoglycemic clamp) will be used as the primary measurement of the neuroendocrine response to hypoglycemia.

Rigor and Reproducibility—Our project has a high degree of scientific rigor and reproducibility as demonstrated by: 1) Our interventions are well established and commonly used in other basic and clinical studies investigating the impact of recurrent hypoglycemia on glucose counter-regulation. 2) A-priori sample size calculation with a high level of scientific power (>80%) derived from the studies and existing literature. 3) Experimental endpoints are measured using established standard operating procedures and highly trained personnel. 4) Statistical analyses are conducted by an experienced biostatistician who is not involved in the collection of experimental outcomes and according to a pre-specified statistical plan.

C. Relevance: Without wishing to be bound by theory, this project will advance scientific knowledge of the mechanisms by which exposure to iatrogenic hypoglycemia leads to pathologically reduced hypoglycemic counter-regulation. The project's translational impact is clearly shown by our focus on evaluating leptin as a treatment for HAAF. Human recombinant leptin, Metreleptin, is a FDA-approved treatment for dyslipidemia, and thus could readily be repurposed to treat HAAF.

D. Experimental Plans

Positive results in either the basic or clinical experiments can be utilized as strong data supporting follow-up studies. For example, focusing on determining the site of actions, either central or peripheral, involved in hypoleptinemia-induced loss of counter-regulation as well as the mechanisms driving the effect. These experiments would utilize the numerous commercially available transgenic mouse models that target leptin expression or signaling. Clinical projects would focus on validating the efficacy of leptin treatment to prevent or reverse compromised glucose counter-regulation.

### REFERENCES

1. Cryer, P. E., Mechanisms of Hypoglycemia-Associated Autonomic Failure in Diabetes. N Engl J Med, 2013. 369(4): p. 362-72 DOI: 10.1056/NEJMra1215228.
2. Cryer, P. E., The Barrier of Hypoglycemia in Diabetes. Diabetes, 2008. 57(12): p. 3169-3176 DOI: 10.2337/db08-1084.
3. Segel, S. A., D. S. Paramore, and P. E. Cryer, Hypoglycemia-Associated Autonomic Failure in Advanced Type 2 Diabetes. Diabetes, 2002. 51(3): p. 724-733 DOI: 10.2337/diabetes.51.3.724.
4. Hopkins, D., et al., Improved biomedical and psychological outcomes 1 year after structured education in flexible insulin therapy for people with type 1 diabetes: the U.K. DAFNE experience. Diabetes care, 2012. 35(8): p. 1638-42 DOI: 10.2337/dc11-1579.
5. Geddes, J., et al., Prevalence of impaired awareness of hypoglycaemia in adults with Type 1 diabetes. Diabetic medicine: a journal of the British Diabetic Association, 2008. 25(4): p. 501-4 DOI: 10.1111/j.1464-5491.2008.02413.x.
6. McCoy, R. G., et al., Increased Mortality of Patients With Diabetes Reporting Severe Hypoglycemia. Diabetes care, 2012. 35(9): p. 1897-1901 DOI: 10.2337/dc11-2054.
7. Skrivarhaug, T., et al., Long-term mortality in a nationwide cohort of childhood-onset type 1 diabetic patients in Norway. Diabetologia, 2006. 49(2): p. 298-305 DOI: 10.1007/s00125-005-0082-6.
8. Feltbower, R. G., et al., Acute complications and drug misuse are important causes of death for children and young adults with type 1 diabetes: results from the Yorkshire Register of diabetes in children and young adults. Diabetes care, 2008. 31(5): p. 922-6 DOI: 10.2337/dc07-2029.
9. Jacobson, A. M., et al., Long-term effect of diabetes and its treatment on cognitive function. The New England journal of medicine, 2007. 356(18): p. 1842-52 DOI: 10.1056/NEJMoa066397.
10. Yeoh, E., et al., Interventions That Restore Awareness of Hypoglycemia in Adults With Type 1 Diabetes: A Systematic Review and Meta-analysis. Diabetes care, 2015. 38(8): p. 1592-609 DOI: 10.2337/dc15-0102.
11. Senthilkumaran, M., X. F. Zhou, and L. Bobrovskaya, Challenges in Modelling Hypoglycaemia-Associated Autonomic Failure: A Review of Human and Animal Studies. International journal of endocrinology, 2016. 2016: p. 9801640 DOI: 10.1155/2016/9801640.
12. Moheet, A., et al., Hypoglycemia-Associated Autonomic Failure in Healthy Humans: Comparison of Two vs Three Periods of Hypoglycemia on Hypoglycemia-Induced Counterregulatory and Symptom Response 5 Days Later. Journal of Clinical Endocrinology & Metabolism, 2014. 99(2): p. 664-670 DOI: 10.1210/jc.2013-3493.
13. Beall, C., M. L. Ashford, and R. J. McCrimmon, The physiology and pathophysiology of the neural control of the counterregulatory response. American Journal of Physiology-Regulatory Integrative and Comparative Physiology, 2012. 302(2): p. R215-R223 DOI: 10.1152/ajpregu.00531.2011.
14. Goldstein, J. L., et al., Surviving starvation: essential role of the ghrelin-growth hormone axis. Cold Spring Harbor symposia on quantitative biology, 2011. 76: p. 121-7 DOI: 10.1101/sqb.2011.76.010447.
15. Watford, M., Starvation: Metabolic Changes, in eLS. 2015, John Wiley & Sons, Ltd DOI: 10.1002/9780470015902.a0000642.pub2.
16. McDougal, D. H., et al., Glial acetate metabolism is increased following a 72-h fast in metabolically healthy men and correlates with susceptibility to hypoglycemia. Acta diabetologica, 2018. 55(10): p. 1029-1036 DOI: 10.1007/s00592-018-1180-5.
17. Gulanski, B. I., et al., Increased Brain Transport and Metabolism of Acetate in Hypoglycemia Unawareness. Journal of Clinical Endocrinology & Metabolism, 2013. 98(9): p. 3811-3820 DOI: 10.1210/jc.2013-1701.
18. Adamson, U., P. E. Lins, and V. Grill, Fasting for 72 h decreases the responses of counterregulatory hormones to insulin-induced hypoglycaemia in normal man. Scandinavian journal of clinical and laboratory investigation, 1989. 49(8): p. 751-6.
19. McDougal, D. H., Acute Caloric Restriction Leads to Loss of Hypoglycemic Counter-Regulation in Mice following Short-Term Refeeding. Diabetes, 2018. 67(Supplement 1): p. 205-OR DOI: 10.2337/db18-205-0R.
20. Reno, C. M., Y. Ding, and R. Sherwin, Leptin acts in the brain to influence hypoglycemic counterregulation: disparate effects of acute and recurrent hypoglycemia on glucagon release. American journal of physiology. Endocrinology and metabolism, 2015. 309(12): p. E960-7 DOI: 10.1152/ajpendo.00361.2015.
21. Ma, Y., et al., Recurrent hypoglycemia inhibits the counterregulatory response by suppressing adrenal activity. The Journal of clinical investigation, 2018. 128(9): p. 3866-3871 DOI: 10.1172/JCI91921.
22. Davis, S. N., et al., Leptin in Acute Stress, in Leptin: Regulation and Clinical Applications, M. D. S. Dagogo-Jack, Editor. 2015, Springer International Publishing: Cham. p. 103-115 DOI: 10.1007/978-3-319-09915-6_9.
23. Sandoval, D. A. and S. N. Davis, Leptin: metabolic control and regulation. Journal of Diabetes and Its Complications, 2003. 17(2): p. 108-13.

### Example 4

Abstract

Hypoglycemia-associated autonomic failure (HAAF) is a maladaptive failure in glucose counterregulation known to be caused by recurrent exposure to insulin-induced hypoglycemia in persons with diabetes. In this study we tested whether exposure to severe caloric restriction can cause a HAAF-like condition in mice. We also tested whether leptin treatment could prevent the development of this condition in mice and the development of HAAF in rats. Mice were placed on 60% caloric restriction (CR) for six consecutive days and then refed for up to four days (CR mice). Rats were exposed to three days of recurrent hypoglycemia (3dRH rats). A sub-set of CR mice and 3dRH rats were given leptin during the CR or 3dRH periods. Following one day of refeeding, CR mice displayed defects in hypoglycemic counterregulation, indicated by significantly lower hypoglycemia-induced glucagon levels relative to controls, 14 pmol/L (SD 11) versus 65 pmol/L (SD 45) (p=0.002). In contrast, glucagon levels in leptin treated CR mice were similar to controls at the same time point, 91.7 pmol/L (SD 20.8) versus 78.1 pmol/L (SD 16.7) (p=0.764). 3dRH rats displayed defects in hypoglycemia-induced epinephrine levels relative to controls, 110.1 ng/mL (SD 23.5) versus 23.5 ng/mL (SD 4.5) (p=0.030), while 3dRH rats treated with leptin had epinephrine levels similar to controls 87.2 ng/mL (SD 19.1) (p=0.522). These findings indicate that 60% CR is a useful model to validate the mechanisms underlying the development of HAAF, while identifying hypoleptinemia as a necessary signaling event driving the development of HAAF.

### INTRODUCTION

Patients with diabetes are vulnerable to hypoglycemia, and exposure to hypoglycemia is associated with an increased risk of all-cause mortality in these patients (22). Patients with longstanding diabetes also often experience recurrent hypoglycemic episodes due to poorly regulated hyperinsulinemia and inadequate glucagon responses. Recurrent bouts of hypoglycemia lead to the development of hypoglycemia associated autonomic failure [(HAAF) (9, 25, 42)], which is characterized by critically reduced epinephrine responses to hypoglycemia as well as hypoglycemia unawareness.

Although HAAF is commonly experienced by persons with diabetes on insulin therapy, diabetes is not required for its development. It is well established that HAAF can be induced in metabolically heathy humans and rodents via recurrent exposure to insulin-induced hypoglycemia (31, 44). Thus, HAAF represents a pathophysiological adaptation brought about by repetitive exposure to hypoglycemia irrespective of disease state. Furthermore, the known antecedent of HAAF, insulin-induced hypoglycemia, is a relatively modern phenomenon which has only arisen following the adoption of exogenous insulin therapy as a treatment for diabetes. From an evolutionary perspective, this form of hypoglycemia would be exceedingly rare and unlikely to drive such distinct and ubiquitous physiological adaptations (3). In fact, glucose homeostasis is so well controlled in mammals that hypoglycemia is rarely experienced except during prolonged starvation (14, 48).

The physiological responses to starvation are similar to those associated with HAAF and include changes in physiology and cerebral metabolism. As glucose stores are depleted during starvation, the brain transitions to relying almost exclusively on ketone bodies, monocarboxylate metabolites produced by the liver, to meet its energy requirements (34, 43). Ketogenic diets cause alterations in hypoglycemic counterregulation in both mice (32) and humans (40), and are associated with increased frequency of hypoglycemia in diabetes patients on insulin therapy (23). Both ketogenic diets and starvation also lead to upregulation of the cerebral metabolism of acetate in humans (4, 28) and rodents (30), which is strongly associated with the development of HAAF (15). In addition, starvation induces a hypoleptinemic state in rats (36) and severe hypoleptinemia has been observed in a rat model of HAAF (41).

Starvation and its physiological antecedents are also associated with a HAAF-like condition. The counterregulatory response to insulin-induced hypoglycemia is significantly blunted directly following a 72-h fast in humans (1). Prolonged caloric restriction dramatically reduces hepatic glycogen content in both humans and animal models (45, 48), and depletion of hepatic glycogen, independent of fasting, leads to deficits in hypoglycemic counterregulation in dogs (49). Thus, it is not clear whether starvation-induced deficits in hypoglycemic counterregulation are due to fasting-induced metabolic adaptations or simply driven by significant reduction of hepatic glycogen stores caused by 72 h of fasting. Furthermore, the fasted state itself induces mild activation of the counterregulatory response, e.g. hyperglucagonemia, independent of overt hypoglycemia (18, 28). This phenomenon further complicates the study of starvation-induced changes in hypoglycemic counterregulation.

In order to address this complication and to further test the association between starvation and the development of a HAAF-like condition, the current study was undertaken to determine if prolonged fasting leads to changes in hypoglycemic counterregulation following refeeding. Assessment of hypoglycemic counterregulation in the refed state eliminates the confounding influences of starvation-induced reductions in hepatic glycogen content and hyperglucagonemia. Furthermore, it is well established that the neuroendocrine signs of HAAF peak 1-2 days following recurrent exposure to insulin-induced hypoglycemia (31, 44). Thus, if starvation is a natural antecedent to HAAF, deficits in hypoglycemic counterregulation should be manifest during post-starvation refeeding.

Therefore, mice exposed to starvation may exhibit a HAAF-like condition during acute refeeding. We utilized a well-established mouse model of starvation, 60% caloric restriction (14, 26, 51), for six days and then assessed counterregulation via a hypoglycemic insulin tolerance test (ITT) on the first, second, and fourth day of refeeding. We also sought to establish a link between the physiological perturbations caused by starvation, e.g. hepatic glycogen depletion or hypoleptinemia, and any observed deficits in hypoglycemic counterregulation as well as the development of HAAF in the rat model.

Methods

Ethical Approval

All animal experiments were approved by the Institutional Animal Care and Use Committee at Pennington Biomedical Research Center [(PBRC); (Approval numbers 981P and 1058P)]. All animals were reared in an AAALAC (Assessment and Accreditation of Laboratory Animal Care) accredited animal facility in accordance with all conditions specified by the United States Department of Agriculture's Office of Laboratory Animal Welfare.

Animals

Male and female C57BL/6J wild-type mice 8-12 weeks of age (PBRC breeding colony) and male Sprague Dawley rats 8-14 weeks of age (Envigo) were used in these studies. All animals were single-housed with a 12-h light and 12-h dark cycle, with the dark cycle beginning at either 18:00 or 19:00. All animals were fed Purina 5001 Rodent Laboratory Chow. Purified tap water was provided ad libitum. For mice, following completion of experiments, animals were euthanized via hypoxia induced by a gradual increase in CO2, followed by exsanguination (adjunctive method). For hypoxia, animals were maintained in their home cages which were placed in a chamber that contained an inflow of CO2 supplied from a compressed gas cylinder at a displacement rate of 20% of the chamber volume per minute. All rats were euthanized via overdose (1.5 mL/kg) with commercial euthanasia solution (Euthasol, 390 mg/mL sodium pentobarbital+50 mg/mL sodium phenytoin; Med-Vet, Mettawa, Ill.) followed by exsanguination (adjunctive method).

Severe Caloric Restriction and Refeeding Paradigm (Experiments 1-4)

A total of 149 mice were subjected to the severe caloric restriction and refeeding paradigm across four separate experiments. The following procedures were identical across all four experiments. Animals were randomly assigned to one of two groups: Mice exposed to severe caloric restriction for six days (CR mice) or ad libitum fed mice (Ad-lib mice). Littermates were assigned in equal numbers to each group and began the paradigm concurrently. See top border of FIG. 11 for graphical representation of the experimental timeline. Following a five-day acclimation period to wire-bottomed cages (Days 0-4), food intake was monitored daily in each mouse for six days prior to initiation of caloric restriction, and each mouse's average daily food intake was determined (Days 5-10). CR mice were then fed 40% of their daily average food intake for six days (Days 11-16) while Ad-lib mice continued to have unrestricted access to food. Mice were fed at 1.5 h prior to lights off each day. Body weight and free-living blood glucose were measured daily immediately prior to feeding. During the six days of CR, mice were administered 1.0 mL of warmed saline subcutaneously to prevent dehydration immediately following body weight and blood glucose measurements. After six days of restriction, the CR mice entered a refeeding period and returned to ad libitum access to food for up to four days, Days 17-20 (FIG. 11, top x-axis), which encompassed Days 0-4 of the refeeding period (FIG. 11, bottom x-axis). During the refeeding period, food intake, free-living blood glucose, and body weight continued to be measured daily at 1.5 h before lights off in both CR and Ad-lib mice up to the terminal day of the experiments.

In addition to the above procedures, the following procedures were performed in four separate experimental cohorts. For Experiment 1, a subset of male CR (n=32) and Ad-lib (n=32) mice were euthanized for determination of blood hormones and metabolites as well as hepatic glycogen concentration on Days 0, 1, 2, and 4 of the refeeding period (n=5-8/group). For Experiment 2, a separate subset of male CR (n=24) and Ad-lib (n=28) mice underwent a hypoglycemic ITT on Days 1, 2, or 4 of the refeeding period (n=6-8/group) for determination of hypoglycemia-induced glucagon (Days 1, 2, and 4) and corticosterone secretion (Day 1 only), as well as serum leptin and insulin levels (n=5-7/group). Experiment 3, a sub-set of female CR (n=4) and Ad-lib (n=4) mice underwent a hypoglycemic ITT on Day 1 of the refeeding period for determination of hypoglycemia-induced glucagon secretion. For Experiment 4, a separate sub-set of male CR (n=19) and Ad-lib (n=6) mice were used. One group of CR mice (n=5) was euthanized on third day of the caloric restriction period (Day 13) for determination of serum leptin levels. The remaining CR and Ad-lib mice received twice daily intraperitoneal (IP) injections of either leptin or PBS during the caloric restriction period (Days 11-16). Injections were made at ≈1.5 h following lights on and 0.5 h before lights off All Ab-lib mice and one group of CR mice (CR+PBS, n=6) received PBS (10 μL/g, IP), while another group of CR mice (CR+Leptin, n=8) received IP leptin during the same time period using the following dosing schedule: 0.5 μg/gram body weight on Days 11-13, and 1 μg/gram body weight on Days 14-16. All three groups of mice were then subjected to a hypoglycemic ITT on Day 1 of the refeeding period (Day 17 relative to the start of the CR paradigm) for determination of hypoglycemia-induced glucagon and corticosterone secretion.

Three Day Recurrent Hypoglycemia Paradigm (Experiment 5)

For Experiment 5, a total of 21 rats were subjected to a three day recurrent hypoglycemia (3dRH) paradigm adapted from previous studies (44). Animals were randomly assigned to one of two groups, 3dRH or Control. 3dRH rats (n=14) received IP insulin injections on three consecutive days at a dose of 10 U/kg, 9 U/kg and 8 U/kg on the first, second, and third days, respectively, at ≈1-2 h following lights on. Control rats (n=7) received PBS instead of insulin at the same time of day during the same three consecutive days. Blood glucose was measured immediately prior to, and at 40 min intervals for 180 min following insulin or PBS injection on each day of the paradigm. Food was removed just prior to insulin or PBS injection and returned 180 min later. Insulin treated rats that did not begin eating within 20 min of being refed were administered 20% dextrose IP in order to raise blood glucose sufficiently to support feeding behavior. On each of the three days of the paradigm, all rats received an IP injection of either PBS or leptin at ≈1.5 h prior to lights off. All control rats and one group of 3dRH rats (3dRH+PBS, n=7) received PBS (1 μL/g), while another group of 3dRH rats (3dRH+Leptin, n=7) received leptin (0.5 mg/mL in PBS) during the same time period using the following dosing schedule: 0.33 mg/kg, 0.5 mg/kg, and 0.66 mg/kg on the first, second, and third days, respectively. On the fourth day, all three cohorts of rats were then subjected to a hypoglycemic ITT for determination of hypoglycemia-induced epinephrine, glucagon, and corticosterone secretion. Leptin levels were also assayed in each group at the conclusion of the ITT.

Body Composition

Body composition was measured in CR and Ad-lib mice from Experiments 1 and 2 (n=52/group) at baseline (Day 10), on the final day of caloric restriction (Day 16), and on the first, second, and fourth day of the refeeding period (Days 17, 18, and 20) via nuclear magnetic resonance spectroscopy NMR (Bruker LF110 BCA-Analyzer, Billerica, Mass.).

Hormones, Metabolites, and Hepatic Glycogen

For Experiment 1, serum hormones and metabolites, as well as hepatic glycogen content were measured following a 4-5 h fast, at ≈13:30. For all other mice and rat experiments (Experiments 2-5), hormones were assayed from blood collected 60 min following insulin administration as part of a hypoglycemic ITT. For mice, trunk blood was collected following CO2 euthanasia. For rats, blood was collected via cardiac puncture following administration of euthanasia solution. For glucagon, leptin, insulin, and β-hydroxybutyrate (BHB) analysis, trunk blood was collected into a 1.7 mL micro-centrifuge tubes and allowed to clot at room temperature for 15 min. Samples were then centrifuged at 4,400 RPM at 4° C. for 10 min. Serum was collected and stored at −80° C. until analysis. For ghrelin and epinephrine assays, blood was collected into micro-centrifuge tubes containing EDTA only for epinephrine, or EDTA plus and 2 μL of the enzyme inhibitor MAFP (Cayman Chemical Ann Arbor, Mich.) for ghrelin. Samples were centrifuged at 2,500 rpm at 4° C. for 15 min. Plasma was collected and stored at −80° C. until analysis. Serum glucagon concentrations were determined by ELISA (10-1281-01, Mercodia, Winston Salem, N.C.). Serum corticosterone concentrations were determined by ELISA (K014-H1, Arbor Assays, Ann Arbor, Mich.). Plasma epinephrine concentrations were determined by ELISA (KA1882, Abnova, Taipei, Taiwan). Plasma ghrelin, serum leptin, and insulin concentrations in mice were determined by ELISA (EZGRA-90K, EZML-82K, and EZRMI-13K, respectively, MilliporeSigma, St. Louis, Mo.). For mouse leptin, values that read as undetectable were replaced with the lowest detectable limit of the assay, 0.02 ng/mL. Rat leptin levels were determined by ELISA (EZRL-83K, MilliporeSigma, St. Louis, Mo.). Serum BHB concentration was determined by colorimetric assay (700190, Cayman Chemical, Ann Arbor, Mich.). Hepatic glycogen concentration was determined by colorimetric assay (Ab169558, Abcam, Cambridge, Mass.). Terminal, fasting, and free-living blood glucose measurements were made by hand held glucometer, Contour Next EZ (Bayer), via tail nick. All fasting and free-living glucose measurements in Experiment 1, 2, 3, and 4 were determined by the average of three concurrent tail glucose reading in each mouse. In contrast, the blood glucose measurements made during the hypoglycemic ITTs associated with Experiments 3, 4, and 5 consisted a single measurement in each animal at each time point.

Hypoglycemic ITT (Experiments 2, 3, 4, and 5)

For Experiments 2, 3 and 4, after a 4-5 h fast mice received an IP injection of insulin, (Humulin R, Lilly USA Indianapolis, Ind.) at a variable dose (1.6-2.3 U/kg) in order to induce hypoglycemia (blood glucose: 40-60 mg/dL) for 30 min. Blood glucose levels were determined via tail nick using a hand held glucometer at −15, 0, 10, 20, 30, 45, and 60 min relative to insulin administration. At the 60-minute time point all animals were euthanized by CO2 inhalation followed by rapid decapitation. Trunk blood was collected for hormone measurements as described above.

For Experiment 5, following an overnight fast (≈14 h) rats received 10 U/kg of insulin IP (Humulin R, Lilly USA Indianapolis, Ind.). Blood glucose levels were determined via tail nick using a hand-held glucometer just prior to, and 60 min following insulin administration. At the 60-minute time point all animals were euthanized with euthanasia solution, as described above, and blood was collected via cardiac puncture for hormone measurements as described above.

Data Analysis

All data were analyzed with GraphPad Prism 8 (GraphPad Software, San Diego, Calif.). Data are expressed as mean (SD). For all statistic tests, a p-value ≤0.05 was selected a priori as the threshold for statistical significance. Correlational analyses were performed using a simple linear regression model. For Experiments 1, 2, and 3, data were analyzed using two-way ANOVAs with two levels of diet, CR and ad libitum, and multiple levels of time depending on the analyses. In situations where data were not collected at multiple time points, unpaired t-tests were used (e.g. hormone measurements in Experiment 3). When two-way ANOVAs were used, differences between the CR and Ad-Lib groups at each time point were determined via Bonferroni's multiple comparison tests.

For Experiments 4 and 5, data were analyzed via two-way ANOVA (blood glucose) or one-way ANOVA (hormones) with two experimental groups (CR+PBS and CR+leptin mice or 3dRH+PBS and 3dRH+leptin in Experiments 3 and 5, respectively) and one control group (either Ad-lib mice or Control rats in Experiments 3 and 5, respectively). For blood glucose data, differences between groups were determined via Bonferroni's multiple comparison tests comparing all three groups at each time point. For hormone data, differences between groups were determined via Bonferroni's multiple comparison tests comparing the two experiments groups to their respective control group.

Results

Physiological response to severe caloric restriction (Experiments 1, 2 and 3)

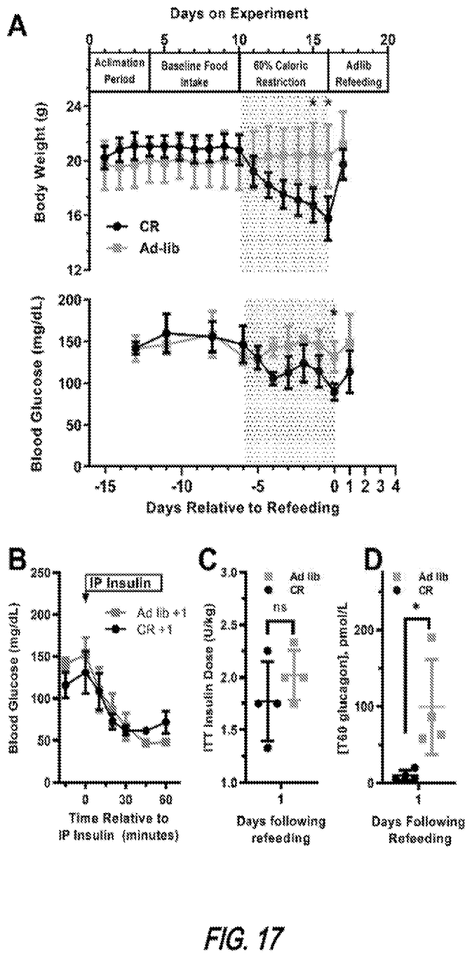

Male CR and Ad-lib mice had nearly identical body weights during the 10-day acclimation and baseline food intake portion of the experiment, with daily means ranging between 24.7-25.4 g and 24.7-25.2 g, respectively. Within one day of exposure to severe caloric restriction, male CR mice had significantly lower mean body weight compared to male Ad-lib mice, 22.9 g (SD 1.6) and 25.3 g (SD 1.7), respectively [(p<0.001); (FIG. 11, top panel)]. The mean body weights of male CR mice continued to diverge from male Ad-lib mice as the CR paradigm continued, reaching a maximum difference on the final day of restriction, 18.9 g (SD 1.6) and 24.9 g (SD 1.6), respectively (p<0.001). Body weights of female CR and Ad-lib mice showed a similar pattern during the severe caloric restriction paradigm (FIG. 17A, top panel).

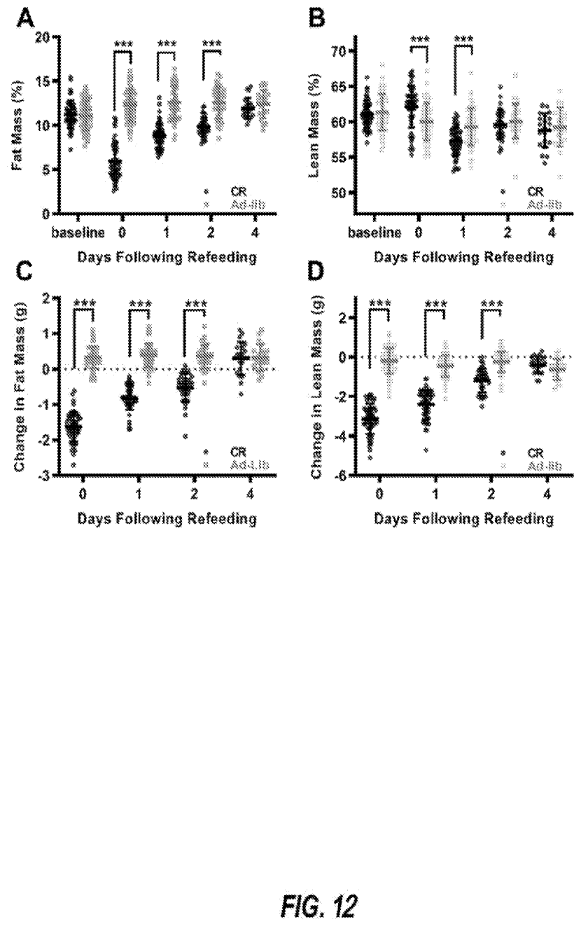

Male CR and Ad-lib mice also had nearly identical body composition at baseline with mean % fat mass of 11.3% (SD 1.6) and 11.1% (SD 1.6), respectively, and mean % lean mass of 61.1% (SD 1.9) and 61.4% (SD 2.5), respectively (FIGS. 12A and 2B). On the final day of caloric restriction male CR mice displayed an average reduction in fat mass of 1.6 g (SD 0.4) and lean mass of 3.1 g (SD 0.8), which was significantly different from male Ad-lib mice [(both p<0.001); (Day 0, FIGS. 12C and 12D)]. This represented an approximately 50% reduction in % fat mass, from 11.3% (SD 1.6) to 5.9% (SD 2.1) (FIG. 12A, Day 0). Due to this dramatic reduction in body fat, male CR mice displayed an increase in average % lean mass on the final day of caloric restriction, which was significantly higher than that of male Ad-lib mice, 62.2% (SD 2.9) versus 60.0% (SD 2.5), respectively [(p<0.001); (FIG. 12B, Day 0)].

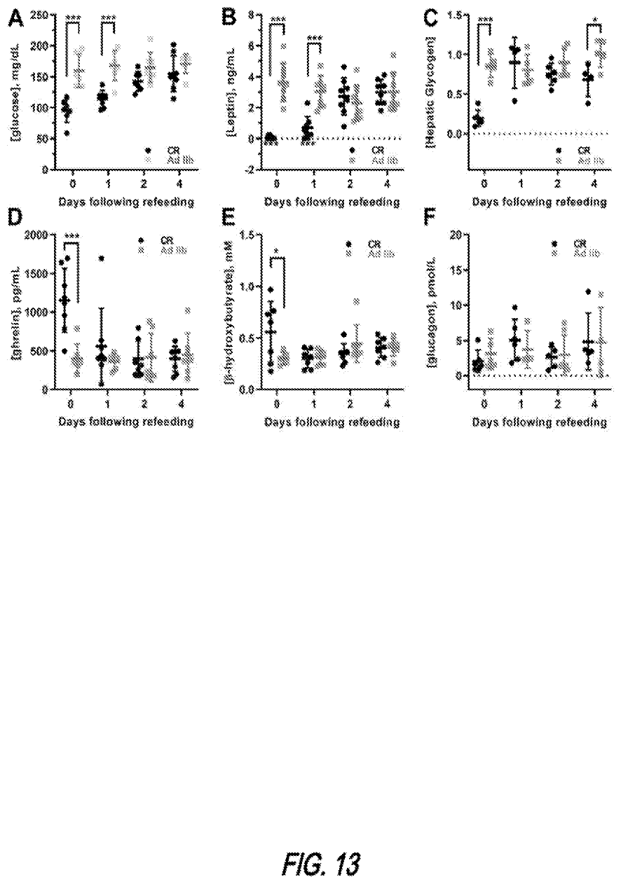

Mean free-living blood glucose values showed a similar pattern to body weight during the acclimation and baseline periods of the experiment, with male CR and Ad-lib mice having similar mean glucose values ranging from 150-161 mg/dL and 151-158 mg/dL respectively. Within two days of exposure to the severe caloric restriction, CR mice displayed lower mean free-living glucose compared to Ad-lib mice, 118 mg/dL (SD 19) versus 157 mg/dL (SD 23), respectively [(p<0.001); (FIG. 11, middle panel)]. Mean free-living glucose of male CR mice continued to diverge relative to Ad-lib mice for the remainder of the caloric restriction period, reaching a nadir on the final day, 83 mg/dL (SD 22) versus 157 mg/dL (SD 18), respectively (p<0.001). Mean fasting blood glucose was also reduced in CR mice relative to Ad-lib mice on the final day of the caloric restriction, 95 mg/dL (SD 19) versus 160 mg/dL (SD 27), respectively [(p<0.001); (FIG. 13A, Day 0)]. Mean free-living glucose of female CR mice showed a similar pattern (FIG. 17A, bottom panel), reaching a nadir on the final day of the caloric restriction period relative to female Ad-lib mice, 89 mg/dL (SD 10) versus 132 mg/dL (SD 18), respectively (p<0.026).

Fasted hepatic glycogen concentrations and serum leptin concentrations were also significantly reduced in CR mice relative to Ad-lib mice on the final day of caloric restriction, 0.20 μg/mL (SD 0.10) versus 0.86 μg/mL (SD 0.14) and 0.07 ng/mL (SD 0.09) versus 3.6 ng/mL (SD 1.2), respectively [(both p<0.001); (FIGS. 13B and 13C, Day 0)]. Mean plasma ghrelin and serum BHB levels were significantly higher in CR mice relative to Ad-lib mice on the final day of the caloric restriction, 1150 pg/mL (SD 410) versus 400 pg/mL (SD 190) and 0.56 mM (SD 0.30) versus 0.30 mM (SD 0.06), respectively [(p<0.001 and p=0.017); (FIGS. 13D and 13E, Day 0)], while mean fasting glucagon levels were not significantly different [(p>0.999); (FIG. 13F)]. CR and Ad-lib mice had nearly identical food intake during the baseline food intake period of the experiment, with daily means ranging between 4.4-3.8 g and 4.5-3.9 g, respectively (FIG. 11, bottom panel).

Physiological Responses to Refeeding Following Severe Caloric Restriction (Experiments 1, 2, and 3)

Following the sixth day of caloric restriction, male and female CR mice were given free access to food for up to four days (refeeding period). Mean body weight of male CR mice remained significantly lower than male Ad-lib mice during the first two days of the refeeding period (p<0.001 and p=0.037, respectively), before normalizing on the third and fourth day of refeeding (FIG. 11, top panel). Although mean body weight of female CR mice was numerically lower that female Ad-lib mice on the first day of refeeding, this difference was not statistically significant (FIG. 17A, top panel).

Relative to male Ad-lib mice, male CR mice had significant reductions in average fat mass and % fat mass on the first two days of refeeding (p<0.001 for all 4 comparisons). These parameters normalized on the fourth day of refeeding (FIGS. 12A and 12C). Similarly, male CR mice had significant reductions in average lean mass on the first two days of refeeding relative to male Ad-lib mice (both p<0.001), before normalizing on the fourth day of refeeding (FIG. 12D). The average % lean mass of male CR mice was also significantly different than male Ad-lib mice on the first day of refeeding (p<0.001), before normalizing on the second and fourth days (FIG. 12B). This restoration of body weight and body composition was accompanied by a significant-increases in mean daily food intake in male CR mice relative to male Ad-lib mice on each of the four days of the refeeding period [(p<0.001 for all 4 comparisons); (FIG. 11, lower panel)].

Mean free-living and mean fasting blood glucose levels of CR mice were both significantly lower than Ad-lib mice on the first day of the refeeding period [(both p<0.001); (FIG. 11, middle panel, FIG. 13A)]. In contrast, average free-living glucose of female CR mice was not significantly different from female Ad-lib mice on the first day of refeeding [(p=0.121); (FIG. 17A, bottom panel)]. Although average free-living and fasting blood glucose levels of male CR mice remained numerically lower than Ad-lib mice for the remaining 3 days of the refeeding period, none of these comparisons reached statistical significance (FIG. 11, middle panel; FIG. 13A).

Average fasting leptin levels continued to be significantly lower in male CR mice relative to male Ad-lib mice on the first day of refeeding, 0.70 ng/mL (SD 0.73) versus 3.0 ng/mL (SD 1.0), respectively (p<0.001), but were not significantly different on the remaining days of refeeding (FIG. 13B). Mean fasting hepatic glycogen concentrations were similar between male CR and male Ad-lib mice during the refeeding period, although CR mice did have significantly lower levels on the fourth day of the refeeding period [(p=0.044); (FIG. 13C)]. Mean fasting serum ghrelin, glucagon, and BHB concentrations were not significantly different between CR and Ad-lib mice during any of the days of the refeeding period (FIGS. 13D, 13E, and 13F).

Hypoglycemia-Induced Glucagon Secretion Following Exposure to Caloric Restriction (Experiments 2 and 3)

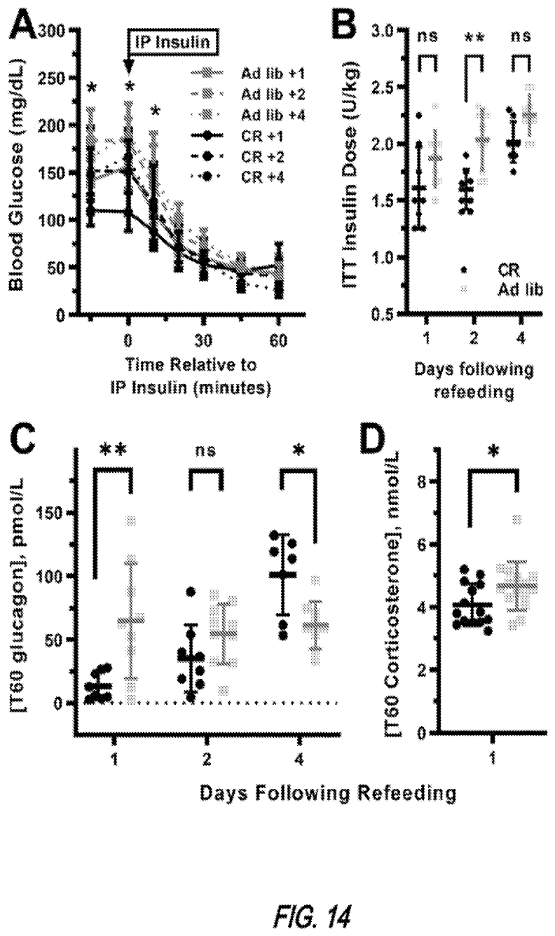

Hypoglycemic counterregulation was assessed via a hypoglycemic ITT in eight groups of mice: one group of male CR mice and one group of male Ad-lib mice on the first, second, and fourth day of the refeeding period (Experiment 2), as well as one group of female CR and Ad-lib mice on the first day of refeeding (Experiment 3). Due to differences in baseline blood glucose levels and insulin sensitivity, both between and within the groups, a variable dose of insulin was administered to achieve similar levels of hypoglycemia during the ITT procedure (FIG. 14A, FIG. 17B). Mean insulin doses in male CR mice were 1.6 U/kg (SD 0.4), 1.6 U/kg (SD 0.2), and 2.0 U/kg (SD 0.2) on the first, second, and fourth day of refeeding, respectively, while mean insulin doses in male Ad-lib mice were 1.9 U/kg (SD 0.3), 2.0 U/kg (SD 0.3), and 2.3 U/kg (SD 0.2) on the first, second, and fourth day of refeeding, respectively. There was no difference in mean insulin dose in male CR and Ad-lib mice on the first and fourth day of refeeding, but insulin dose was reduced in male CR mice (p=0.001) on the second day of refeeding (FIG. 14B). There was no difference in mean insulin dose in female CR and Ad-lib mice (p=0.304, FIG. 17C).

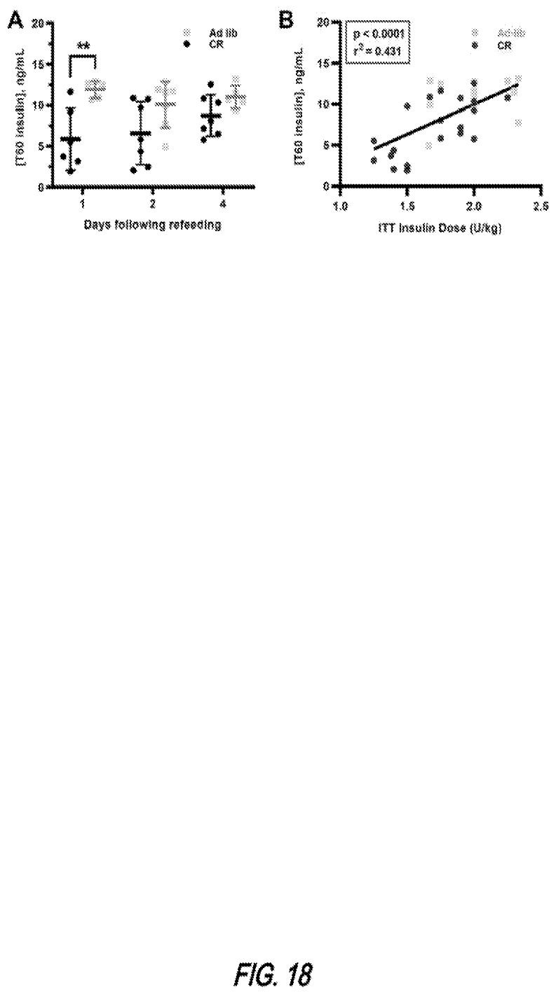

To determine the effect of insulin dose on serum insulin levels, insulin levels were assayed 60 min following insulin administration in a subset of male CR and Ab-lib mice that underwent an ITT (n=37). Mean serum insulin levels in male CR mice were 5.9 ng/mL (SD 3.8), 6.6 ng/mL (SD 3.8), and 8.7 ng/mL (SD 2.5) on the first, second, and fourth day of refeeding, respectively, while serum insulin levels in male Ad-lib mice were 11.9 ng/mL (SD 1.0), 10.1 ng/mL (SD 2.8), and 11.0 ng/mL (SD 1.4) on the first, second, and fourth day of refeeding, respectively. Mean serum insulin levels were significantly reduced in male CR mice relative to male Ad-lib mice on the first day of the refeeding period (p=0.007), but not on the second (p=0.091) and fourth days (p=0.562; FIG. 18A). There was a significant correlation between Insulin dose and serum insulin (p<0.001) with insulin dose displaying a high predictive association with serum insulin levels (r²=0.431; FIG. 18B).

Mean blood glucose at 20, 30, 45, or 60 min post insulin injection was not significantly different between any of the six groups of male mice (FIG. 14A) or the two groups of female mice at any time point (FIG. 17B). The mean blood glucose across all six groups of male mice at 30, 45, and 60 min post insulin injection was 66 mg/dL (SD 9), 47 mg/dL (SD 5), and 41 mg/dL (SD 1), respectively. Trunk blood was collected 60 min post insulin injection and serum levels of glucagon, corticosterone, and leptin were measured. On the first day of the refeeding period male CR mice had significantly reduced hypoglycemia-induced glucagon levels compared to Ad-lib mice, 14 pmol/L (SD 11) versus 65 pmol/L (SD 45), respectively (p=0.002). Female mice showed a similar pattern on the first day of refeeding, with female CR mice having significantly reduced glucagon levels compared to female Ad-lib mice, 10 pmol/L (SD 7) versus 99 pmol/L (SD 62), respectively (p=0.028); (FIG. 17D). There were no differences in glucagon levels in male mice on the second day, but male CR mice had significantly increased hypoglycemia-induced glucagon levels on the fourth day compared to Ad-lib mice, 101 pmol/L (SD 31) versus 61 pmol/L (SD 19), respectively [(p=0.023); (FIG. 14C)].

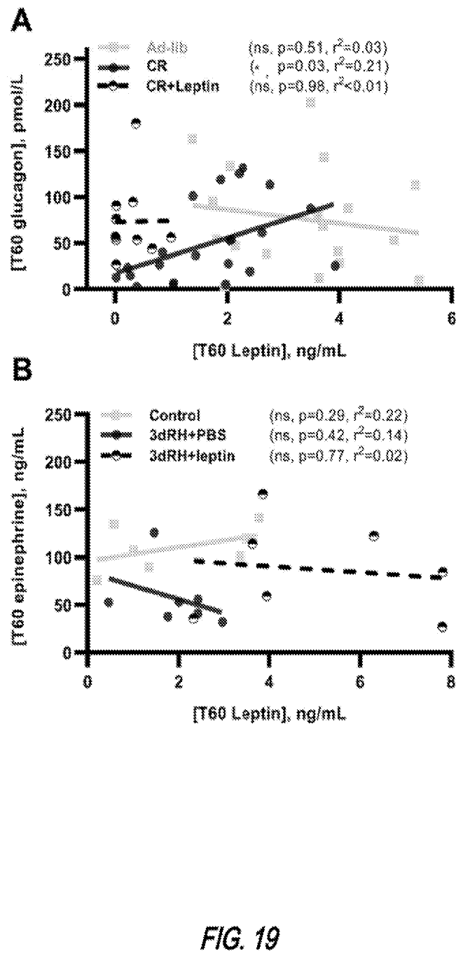

Serum leptin levels were also significantly reduced at the 60-minute time point of the ITT in male CR mice relative to male Ad-lib mice on the first and second day of the refeeding period, 0.9 ng/mL (SD 0.8) versus 3.8 ng/mL (SD 1.1) and 1.9 ng/mL (SD 1.3) versus 4.1 ng/mL (SD 2.0), respectively (p<0.001 and p=0.015, respectively). These reduced leptin levels were significantly correlated with reduced hypoglycemia-induced glucagon release in individual male CR mice [(p=0.031, r²=0.212); (FIG. 19A, CR group)]. Leptin levels were not affected by exposure to the hypoglycemic ITT, as there was no significant within-group differences between mean fasting leptin levels, from Experiment 1 (FIG. 13B), and mean leptin levels measured at the end of the ITT, from Experiment 3, in any of the four groups of mice (CR and Ad-lib mice on Days 1 and 2 of the refeeding period).

Hypoglycemia-Induced Corticosterone Secretion Following Exposure to Caloric Restriction (Experiments 2 and 4)

In order to further quantify starvation-induced effects on hypoglycemic counterregulation, hypoglycemia-induced changes in corticosterone levels were assessed in male mice on the first day of the refeeding period. Given that our method of euthanasia is associated with increased corticosterone release (39), we chose to pool comparable data across Experiments 2 and 4 to increase our statistical power (n=14-15/group). Mice exposed to six days of caloric restriction had significantly reduced corticosterone levels on the first day of refeeding relative to ad libitum fed mice, 4.1 nmol/L (SD 0.7) versus 4.7 pmol/L (SD 0.8), respectively [(p=0.034); (FIG. 14D)].

The Effect of Leptin Treatment on Hypoglycemic Counterregulation Following Exposure to Caloric Restriction (Experiments 4)

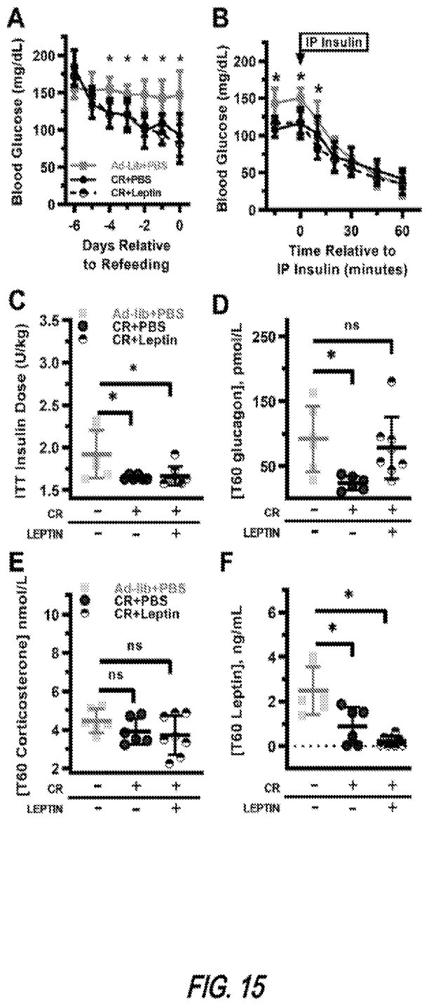

In order to further evaluate the relationship between hypoleptinemia and starvation-induced loss of glucose counterregulation, we tested whether leptin supplementation during the caloric restriction period would reverse the effect. To inform our leptin dosing schedule, serum leptin levels were measured in five CR mice on the third day of the caloric restriction period (Day 13). Mean leptin levels on Day 13 were 1.3 ng/mL (SD 1.0), thus falling half-way between levels measured in CR and Ad-lib mice on the final day of caloric restriction. Blood glucose and hypoglycemic counterregulation were then assessed in three addition groups of male mice: CR+PBS, CR+Leptin, and Ad-lib+PBS. Free-living blood glucose levels during the period of caloric restriction were similar in CR+PBS and CR+Leptin mice, but reduced in both groups relative to Ad-lib mice on the last five days of CR (FIG. 15A). Hormonal responses to hypoglycemia were then assessed on the first day of the refeeding period via a hypoglycemic ITT. Similar to Experiment 2, the level of hypoglycemic exposure during the ITT was equivalent in the three groups (FIG. 15B), but the dose of insulin administered was lower in CR+PBS and CR+Leptin mice relative to Ad-lib+PBS mice [p=0.039 and p=0.040, respectively); (FIG. 15C)]. In contrast, hypoglycemia-induced glucagon levels were similar in Ad-lib+PBS and CR+Leptin mice, 91.7 pmol/L (SD 20.8) and 78.1 pmol/L (SD 16.7), respectively (p=0.764), but significantly reduced to 23.5 pmol/L (SD 4.5) in CR+PBS mice relative to Ad-lib-PBS controls [(p=0.020); (FIG. 15D)]. Hypoglycemia-induced corticosterone levels in both CR groups were not significantly different from controls (FIG. 15E), while serum leptin levels at the conclusion of the ITT were lower in both CR+PBS and CR+Leptin mice relative to Ad-lib+PBS mice [p=0.004 and p<0.001, respectively); (FIG. 15F)]. Similar to Ad-lib mice, there was no correlation between serum leptin and glucagon levels following the ITT in CR+Leptin mice (FIG. 19A).

The Effect of Leptin Treatment on Hypoglycemic Counterregulation Following Exposure to Recurrent Hypoglycemia (Experiments 5)

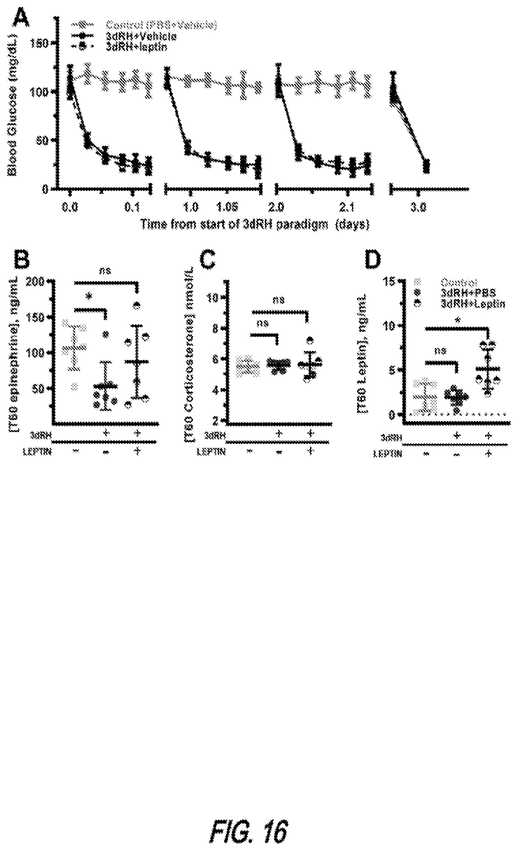

In order to test whether leptin supplementation might prevent the development of HAAF, we employed our leptin dosing strategy in a well-established animal model of HAAF, 3 days of recurrent hypoglycemia [(3dRH); (44)]. Analogous to Experiment 4, three groups were used: 3dRH+PBS, 3dRH+Leptin, and Control rats. Both 3dRH groups experienced nearly identical exposure to hypoglycemia during the 3dRH paradigm, and there was no difference in mean glucose levels at the 60 min time point of the hypoglycemic ITT in all three groups (FIG. 16A). Hypoglycemia-induced epinephrine levels were similar in Control and 3dRH+Leptin rats, 110.1 ng/mL (SD 23.5) and 87.2 ng/mL (SD 19.1), respectively (p=0.522), but significantly reduced to 23.5 ng/mL (SD 4.5) in 3dRH+PBS rats relative to controls [(p=0.030); (FIG. 16B)]. Hypoglycemia-induced corticosterone levels in both 3dRH groups were not significantly different from controls (FIG. 16C). Similarly, hypoglycemia-induced glucagon levels were not significantly different form controls, 34 pmol/L (SD 12), 37 pmol/L (SD 11), and 34 pmol/L (SD 9) in Control, 3dRH+PBS, and 3dRH+Leptin, respectively (p>0.999 for both groups). Serum leptin levels at the conclusion of the ITT were similar in Control and 3dRH+PBS rats, 2.0 ng/mL (SD 1.5) and 1.9 ng/mL (SD 0.8), respectively (p=0.999), but significantly increased to 5.1 ng/mL (SD 2.2) in 3dRH+Leptin rats relative to controls [p=0.004); (FIG. 16D)]. There was no correlation between serum leptin and epinephrine levels following the ITT in any of the three groups (FIG. 19B).

Discussion

(1, 26, 27, 36, 41). In this study we demonstrate that both male and female mice exposed to starvation, via six days of 60% CR, exhibit deficits in their counterregulatory response to insulin-induced hypoglycemia following refeeding, and that the effect is associated with hypoleptinemia. More importantly, we established that normal hypoglycemic counterregulation was rescued by antecedent leptin supplementation in both our CR paradigm and a rat model of HAAF. Our data also adds to the body of knowledge regarding the physiological response to refeeding following exposure to starvation. These findings demonstrate that 60% CR is a useful model to explore the mechanisms underlying the development of HAAF. Furthermore, we identify hypoleptinemia as a necessary signaling event driving the development of HAAF.

Severe caloric restriction paradigms have been routinely used to investigate the physiological response to starvation in both mice (24, 26, 47, 51) and rats (6, 36) via 50-60% CR or a 48-h fast, respectively. The metabolic parameters measured on the final day of our severe caloric restriction are consistent with these prior studies. Yet, little data has been reported on the restoration of pre-starvation physiology during refeeding in rodent models of severe CR, thus our study makes significant contributions to this body of knowledge. Several of the physiological responses to starvation were reversed to control levels within 24 h. These included elevations in ghrelin and BHB, as well as the depletion of hepatic glycogen content. These results are consistent with the known biological regulation of circulating ghrelin (27, 47) and ketone levels (34, 43, 48) and previous measurements of hepatic glycogen following refeeding in 48 h fasted rats (6). In contrast, leptin levels remained lower than controls on the first day of refeeding and this coincided with alterations in glucose homeostasis including reductions in free-living glucose, fasting glucose, as well as a profound reduction in hypoglycemia-induced glucagon secretion, with milder reductions in hypoglycemia-induced corticosterone levels. In addition, individual CR mice which experienced more pronounced hypoleptinemia during the refeeding period displayed more extreme reductions in hypoglycemia-induced glucagon levels during a hypoglycemic challenge, and leptin treatment during caloric restriction restored the deficit in glucagon release. Taken together, these observations indicate a role for hypoleptinemia as a signaling event driving impaired hypoglycemic counterregulation following starvation in mice.

Leptin is a known regulator of energy homeostasis and neuroendocrine function (21) and more recently has gained recognition as a potent regulator of glucose homeostasis (29). Although much of this work has focused on the glucose lowering effect of leptin (7, 37, 50), acute leptin treatment has been shown to influence counterregulation in response to both fasting- and insulin-induced hypoglycemia in rats (36, 41). Perry et al. (36) found that the physiological processes that maintain glucose levels during starvation (48-h fast) in rats, were elicited by hypoleptinemia along with insulinopenia, within 16 h of fasting. These studies also demonstrated that acute leptin infusion during the starvation state had significant effects on blood glucose, with physiological doses reducing blood glucose and supraphysiological doses increasing blood glucose. In contrast, our data indicates that leptin administration during starvation in mice has less profound effects on chronic blood glucose levels, as free-living glucose levels in CR+PBS and CR+Leptin mice were nearly identical during the caloric restriction period. This discrepancy could be due to either species-specific differences or methodological differences between the experimental paradigms. Although it is worth noting that our leptin treatment in rats also had no effect on free-living, fasting, or ITT glucose levels (3dRH+PBS versus 3dRH+Leptin groups), yet this could be explained by the longer interval, 12-16 h, between leptin treatment and any of our blood glucose measurements relative to the work of Perry et al. (36).

The work of Reno et al. (41) which evaluated the effect of acute leptin treatment on counterregulatory responses to concurrent insulin-induced hypoglycemia is also highly relevant to our work. These experiments demonstrated that acute intracerebroventricular (ICV) leptin infusion had mixed effects on the counterregulatory responses of rats exposed to a single bout of hypoglycemia, with glucagon levels being decreased by 50% and epinephrine levels increases by 19%. In addition, acute ICV leptin infusion concurrent with a 90 min hypoglycemic challenge in 3dRH rats increased both glucagon and epinephrine levels, 45% and 25% respectively, during the first 40 min of hypoglycemia relative to 3dRH rats that were not treated with leptin. Yet, epinephrine levels in leptin treated 3dRH rats remained 60% lower than untreated rats exposed to a single bout of hypoglycemia, thus leptin infusion did not correct the deficit in epinephrine release caused by exposure to 3dRH. In contrast, our leptin treatment paradigm, which occurred concurrent with the exposure to antecedent hypoglycemia, as opposed to during the hypoglycemic challenge itself, increased epinephrine levels by 50% relative to untreated 3dRH rats, while also normalizing epinephrine release relative to rats exposed to a single bout of hypoglycemia. Hypoglycemia-induced glucagon levels were not affected by exposure to 3dRH or leptin. Although our glucagon results are not consistent with Reno et al. (41), this can be explained by the known inconsistences of the 3dRH paradigm in reliably producing reductions in glucagon levels (see 44 for discussion of this phenomenon).

Reno et al. (41) also reported that rats exposed to 3dRH had nearly undetectable fasting leptin levels one day following the completion of the 3dRH paradigm, thus demonstrating an association between hypoleptinemia and impaired counterregulation. Although the results of our CR experiments in mice support this observation, we cannot confirm this finding in our rat model because our leptin measurements occurred at the conclusion of the ITT, thus not in a typical fasted state. Insulin administration at the dose used in our ITT has been shown to raise leptin levels in rats (16), which could explain why our leptin levels in control rats were 2-fold higher (˜2 ng/mL) than that commonly reported in fasted Sprague Dawley rats (˜1 ng/mL); (e.g. 2, 36). Also, given the increase in leptin levels in 3dRH+Leptin rats at the conclusion of the ITT, we also cannot exclude that the rescue of their epinephrine responses was driven by increased serum leptin levels as opposed to prior exposure to leptin during the 3dRH paradigm. Although this seems highly unlikely, as the leptin levels of the 3dRH+Leptin rats were comparable to levels in lean fed rats (5), and thus do not represent a supraphysiological elevation of leptin.

Altogether, our findings in the CR and 3dRH models contributes to the understanding of the restoration of normal physiology following starvation and builds on the body of work of Perry et al. (36) and Reno et al. (41) linking leptin and hypoglycemic counterregulation. Our work also significantly moves the field forward by demonstrating a causative link between antecedent exposure to hypoleptinemia and the development of impaired hypoglycemic counterregulation, especially in the context of the development of HAAF. This could have profound implications for the clinical management of patients at risk for HAAF.

Clinical Implications

HAAF is highly prevalent in diabetic patients, dangerous, and difficult to manage clinically. Patients with longstanding diabetes are vulnerable to exposure to recurrent, treatment-induced hypoglycemic episodes, which lead to the development of HAAF and further risk of hypoglycemic complications. It is estimated that 40% of diabetic patients are at high risk for hypoglycemic complications (19), and that 20% have HAAF (13). Various mechanisms underlying the development of HAAF have been proposed, including altered CNS glucose utilization, modified CNS glycogen storage, and alterations in neurotransmission and/or neuromodulation, yet the data supporting each of these mechanisms are largely inconsistent (3, 9, 25). The results of the present study along with Reno et al. (41) indicate that HAAF is caused by antecedent conditions which may also be present during starvation, most notably hypoleptinemia.

The fact that recurrent exposure to hypoglycemia is the primary antecedent of HAAF is well established. Reno et al. (41) showed that exposure to recurrent hypoglycemia in rats causes hypoleptinemia. Our study demonstrates that leptin treatment during recurrent hypoglycemia, directed at preventing hypoleptinemia, blocks the development of HAAF in rats. These results indicate that leptin may have therapeutic value for the treatment or prevention of HAAF in patients with diabetes. Human recombinant leptin, metreleptin, is an FDA-approved therapy for the treatment of generalized dyslipidemia (8, 33), and therefore could readily be repurposed for the treatment or prevention of HAAF.

Before this can occur, two key phenomena must be established in clinical studies. One, the effect of recurrent exposure to hypoglycemia on leptin levels in humans must be studied. Although acute changes in leptin during exposure to hypoglycemia has been investigated with mixed results (see 12 for details), to the best of our knowledge no study has investigated longer-term effects on leptin levels following acute or recurrent hypoglycemia in humans. Secondly, a clinical trial investigating whether leptin treatment during exposure to recurrent hypoglycemia prevents the development of HAAF in non-diabetic humans must be conducted. These studies would lay the groundwork for clinical trials testing the efficacy of leptin treatment in prevention of HAAF and hypoglycemia unawareness in patients with diabetes.

### References Cited in this Example

1. Adamson U, Lins P E, and Grill V. Fasting for 72 h decreases the responses of counterregulatory hormones to insulin-induced hypoglycaemia in normal man. Scand J Clin Lab Invest 49: 751-756, 1989.
2. Bagnasco M, Kalra P S, and Kalra S P. Ghrelin and Leptin Pulse Discharge in Fed and Fasted Rats. Endocrinology 143: 726-729, 2002.
3. Beall C, Ashford M L, and McCrimmon R J. The physiology and pathophysiology of the neural control of the counterregulatory response. Am J Physiol-Regul Integr Comp Physiol 302: R215-R223, 2012.
4. Bluml S, Shic F, Lai C L, Yahya K, Lin A, and Ross B. Glutamate—glutamine cycling in epileptic patients on ketogenic diets. In Proceedings, 10th International Society of Magnetic Resonance in Medicine 1: 417, 2002.
5. Bodosi B, Gardi J, Hajdu I, Szentirmai E, Obal F, Jr., and Krueger J M. Rhythms of ghrelin, leptin, and sleep in rats: effects of the normal diurnal cycle, restricted feeding, and sleep deprivation. American journal of physiology Regulatory, integrative and comparative physiology 287: R1071-1079, 2004.
6. Bois-Joyeux B, Chanez M, and Peret J. Age-dependent glycolysis and gluconeogenesis enzyme activities in starved-refed rats. Diabete Metab 16: 504-512, 1990.
7. Chinookoswong N, Wang J L, and Shi Z Q. Leptin restores euglycemia and normalizes glucose turnover in insulin-deficient diabetes in the rat. Diabetes 48: 1487-1492, 1999.
8. Chou K, and Perry C M. Metreleptin: first global approval. Drugs 73: 989-997, 2013.
9. Cryer P E. Mechanisms of Hypoglycemia-Associated Autonomic Failure in Diabetes. N Engl J Med 369: 362-372, 2013.
10. Davis S N, Dobbins R, Tarumi C, Colburn C, Neal D, and Cherrington A D. Effects of differing insulin levels on response to equivalent hypoglycemia in conscious dogs. The American journal of physiology 263: E688-695, 1992.
11. Davis S N, Goldstein R E, Jacobs J, Price L, Wolfe R, and Cherrington A D. The effects of differing insulin levels on the hormonal and metabolic response to equivalent hypoglycemia in normal humans. Diabetes 42: 263-272, 1993.
12. Davis S N, Lamos E M, Loper H, and Younk L M. Leptin in Acute Stress. In: Leptin: Regulation and Clinical Applications, edited by Dagogo-Jack M D S. Cham: Springer International Publishing, 2015, p. 103-115.
13. Geddes J, Schopman J E, Zammitt N N, and Frier B M. Prevalence of impaired awareness of hypoglycaemia in adults with Type 1 diabetes. Diabet Med 25: 501-504, 2008.
14. Goldstein J L, Zhao T J, Li R L, Sherbet D P, Liang G, and Brown M S. Surviving starvation: essential role of the ghrelin-growth hormone axis. Cold Spring Harb Symp Quant Biol 76: 121-127, 2011.
15. Gulanski B I, De Feyter H M, Page K A, Belfort-DeAguiar R, Mason G F, Rothman D L, and Sherwin R S. Increased Brain Transport and Metabolism of Acetate in Hypoglycemia Unawareness. J Clin Endocrinol Metab 98: 3811-3820, 2013.
16. Hardie L J, Rayner D V, Holmes S, and Trayhurn P. Circulating Leptin Levels Are Modulated by Fasting, Cold Exposure and Insulin Administration in Lean but Not Zucker (fa/fa) Rats as Measured by ELISA. Biochemical and Biophysical Research Communications 223: 660-665, 1996.
17. Heller S R, and Cryer P E. Reduced neuroendocrine and symptomatic responses to subsequent hypoglycemia after 1 episode of hypoglycemia in nondiabetic humans. Diabetes 40: 223-226, 1991.
18. Hojlund K, Wildner-Christensen M, Eshoj O, Skjaerbaek C, Holst J J, Koldkjaer O, Moller Jensen D, and Beck-Nielsen H. Reference intervals for glucose, beta-cell polypeptides, and counterregulatory factors during prolonged fasting. Am J Physiol Endocrinol Metab 280: E50-E58, 2001.
19. Hopkins D, Lawrence I, Mansell P, Thompson G, Amiel S, Campbell M, and Heller S. Improved biomedical and psychological outcomes 1 year after structured education in flexible insulin therapy for people with type 1 diabetes: the U.K. DAFNE experience. Diabetes Care 35: 1638-1642, 2012.
20. Jacobson L, Ansari T, and McGuinness O P. Counterregulatory deficits occur within 24 h of a single hypoglycemic episode in conscious, unrestrained, chronically cannulated mice. Am J Physiol Endocrinol Metab 290: E678-E684, 2006.
21. Kelesidis T, Kelesidis I, Chou S, and Mantzoros C S. Narrative Review: The Role of Leptin in Human Physiology: Emerging Clinical Applications. Annals of Internal Medicine 152: 93-+, 2010.
22. Khunti K, Davies M, Majeed A, Thorsted B L, Wolden M L, and Paul S K. Hypoglycemia and Risk of Cardiovascular Disease and All-Cause Mortality in Insulin-Treated People With Type 1 and Type 2 Diabetes: A Cohort Study. Diabetes Care 2014.
23. Leow Z Z X, Guelfi K J, Davis E A, Jones T W, and Fournier P A. The glycaemic benefits of a very-low-carbohydrate ketogenic diet in adults with Type 1 diabetes mellitus may be opposed by increased hypoglycaemia risk and dyslipidaemia. Diabet Med 2018.
24. Li R L, Sherbet D P, Elsbernd B L, Goldstein J L, Brown M S, and Zhao T J.
Profound hypoglycemia in starved, ghrelin-deficient mice is caused by decreased gluconeogenesis and reversed by lactate or fatty acids. The Journal of biological chemistry 287: 17942-17950, 2012.
25. Lontchi-Yimagou E, You J Y, Carey M, Gabriely I, Shamoon H, and Hawkins M. Potential approaches to prevent hypoglycemia-associated autonomic failure. Journal of Investigative Medicine 66: 641-647, 2018.
26. Mani B K, Osborne-Lawrence S, Vijayaraghavan P, Hepler C, and Zigman J M. betal-Adrenergic receptor deficiency in ghrelin-expressing cells causes hypoglycemia in susceptible individuals. The Journal of clinical investigation 126: 3467-3478, 2016.
27. Mani B K, and Zigman J M. Ghrelin as a Survival Hormone. Trends Endocrinol Metab 28: 843-854, 2017.
28. McDougal D H, Darpolor M M, DuVall M A, Sutton E F, Morrison C D, Gadde K M, Redman L M, and Carmichael O T. Glial acetate metabolism is increased following a 72-h fast in metabolically healthy men and correlates with susceptibility to hypoglycemia. Acta diabetologica 55: 1029-1036, 2018.
29. Meek T H, and Morton G J. The role of leptin in diabetes: metabolic effects. Diabetologia 59: 928-932, 2016.
30. Melo T M, Nehlig A, and Sonnewald U. Neuronal—glial interactions in rats fed a ketogenic diet. Neurochemistry International 48: 498-507, 2006.
31. Moheet A, Kumar A, Eberly L E, Kim J, Roberts R, and Seaquist E R. Hypoglycemia-Associated Autonomic Failure in Healthy Humans: Comparison of Two vs Three Periods of Hypoglycemia on Hypoglycemia-Induced Counterregulatory and Symptom Response 5 Days Later. J Clin Endocrinol Metab 99: 664-670, 2014.
32. Morrison C D, Hill C M, DuVall M A, Coulter C E, Gosey J L, Herrera M J, Maisano L E, Sikaffy H X, and McDougal D H. Consuming a ketogenic diet leads to altered hypoglycemiccounter-regulation in mice. Journal of Diabetes and Its Complications 34: 107557, 2020.
33. Oral E A, Gorden P, Cochran E, Araujo-Vilar D, Savage D B, Long A, Fine G, Salinardi T, and Brown R J. Long-term effectiveness and safety of metreleptin in the treatment of patients with partial lipodystrophy. Endocrine 2019.
34. Owen O E. Ketone bodies as a fuel for the brain during starvation. Biochemistry and Molecular Biology Education 33: 246-251, 2005.
35. Oz G, DiNuzzo M, Kumar A, Moheet A, Khowaja A, Kubisiak K, Eberly L E, and Seaquist E R. Cerebral glycogen in humans following acute and recurrent hypoglycemia: Implications on a role in hypoglycemia unawareness. Journal of cerebral blood flow and metabolism: official journal of the International Society of Cerebral Blood Flow and Metabolism 37: 2883-2893, 2017.
36. Perry R J, Wang Y, Cline G W, Rabin-Court A, Song J D, Dufour S, Zhang X M, Petersen K F, and Shulman G I. Leptin Mediates a Glucose-Fatty Acid Cycle to Maintain Glucose Homeostasis in Starvation. Cell 172: 234-248 e217, 2018.
37. Perry R J, Zhang X M, Zhang D, Kumashiro N, Camporez J P, Cline G W, Rothman D L, and Shulman G I. Leptin reverses diabetes by suppression of the hypothalamic-pituitary-adrenal axis. Nature medicine 20: 759-763, 2014.
38. Poplawski M M, Mastaitis J W, and Mobbs C V. Naloxone, but not valsartan, preserves responses to hypoglycemia after antecedent hypoglycemia: role of metabolic reprogramming in counterregulatory failure. Diabetes 60: 39-46, 2011.
39. Powell K, Ethun K, and Taylor D K The effect of light level, CO2 flow rate, and anesthesia on the stress response of mice during CO2 euthanasia. Lab Animal 45: 386-395, 2016.
40. Ranjan A, Schmidt S, Damm-Frydenberg C, Steineck I, Clausen T R, Holst J J, Madsbad S, and Norgaard K. Low-Carbohydrate Diet Impairs the Effect of Glucagon in the Treatment of Insulin-Induced Mild Hypoglycemia: A Randomized Crossover Study. Diabetes Care 40: 132-135, 2017.
41. Reno C M, Ding Y, and Sherwin R. Leptin acts in the brain to influence hypoglycemic counterregulation: disparate effects of acute and recurrent hypoglycemia on glucagon release. American journal of physiology Endocrinology and metabolism 309: E960-967, 2015.
42. Rogers R C, McDougal D H, and Hermann G E. Hindbrain Astrocyte Glucodetectors and Counterregulation. In: Appetite and Food Intake: Central Control, edited by Harris RBS. Boca Raton (Fla.): CRC Press/Taylor & Francis, 2017, p. 205-228.
43. Rojas-Morales P, Tapia E, and Pedraza-Chaverri J. beta-Hydroxybutyrate: A signaling metabolite in starvation response? Cell Signal 28: 917-923, 2016.
44. Senthilkumaran M, Zhou X F, and Bobrovskaya L. Challenges in Modelling Hypoglycaemia-Associated Autonomic Failure: A Review of Human and Animal Studies. Int J Endocrinol 2016: 9801640, 2016.
45. Soeters M R, Soeters P B, Schooneman M G, Houten S M, and Romijn J A. Adaptive reciprocity of lipid and glucose metabolism in human short-term starvation. Am J Physiol Endocrinol Metab 303: E1397-E1407, 2012.
46. Sprague J E, and Arbelaez A M. Glucose counterregulatory responses to hypoglycemia. Pediatr Endocrinol Rev 9: 463-473; quiz 474-465, 2011.
47. Sun Y, Butte N F, Garcia J M, and Smith R G. Characterization of adult ghrelin and ghrelin receptor knockout mice under positive and negative energy balance. Endocrinology 149: 843-850, 2008.
48. Watford M. Starvation: Metabolic Changes. In: eLSJohn Wiley & Sons, Ltd, 2015.
49. Winnick J J, Kraft G, Gregory J M, Edgerton D S, Williams P, Hajizadeh I A, Kamal M Z, Smith M, Farmer B, Scott M, Neal D, Donahue E P, Allen E, and Cherrington A D. Hepatic glycogen can regulate hypoglycemic counterregulation via a liver-brain axis. The Journal of Clinical Investigation 126: 2236-2248, 2016.
50. Yu X, Park B H, Wang M Y, Wang Z V, and Unger R H. Making insulin-deficient type 1 diabetic rodents thrive without insulin. Proceedings of the National Academy of Sciences of the United States of America 105: 14070-14075, 2008.
51. Zhao T J, Liang G, Li R L, Xie X, Sleeman M W, Murphy A J, Valenzuela D M, Yancopoulos G D, Goldstein J L, and Brown M S. Ghrelin O-acyltransferase (GOAT) is essential for growth hormone-mediated survival of calorie-restricted mice. Proceedings of the National Academy of Sciences of the United States of America 107: 7467-7472, 2010.

### Example 5

### BACKGROUND

Patients with diabetes are vulnerable to hypoglycemia, and exposure to hypoglycemia is associated with an increased risk of all-cause mortality in these patients. Patients with longstanding diabetes also often experience recurrent hypoglycemic episodes due to poorly regulated hyperinsulinemia and inadequate glucagon responses. Recurrent bouts of hypoglycemia lead to the development of hypoglycemia associated autonomic failure (HAAF), which is characterized by critically reduced neuroendocrine responses to hypoglycemia as well as hypoglycemia unawareness. HAAF leaves persons with diabetes vulnerable to life-threatening episodes of severe hypoglycemia and is a significant barrier to the maintenance of healthy plasma glucose levels in both type 1 and 2 diabetes.

HAAF is thought to be caused by maladaptive changes in the central nervous system which are instigated by exposure to recurrent hypoglycemia, however its precise cause is unknown. There are three key observations that demonstrate a link between hypoleptinemia and the development of HAAF. These include: 1) exposure to recurrent hypoglycemia, a known antecedent of HAAF, causes hypoleptinemia in rodents 2) exposure to short-term starvation, a known antecedent of hypoleptinemia, causes HAAF-like symptoms in both mice and humans, 3) the induction of HAAF-like symptoms in mice via starvation can be prevented with acute leptin supplementation during starvation. Thus, without wishing to be bound by theory, acute leptin supplementation can prevent the development of HAAF. This will be validated in the following specific aims:

Aim 1: Determine if exposure to recurrent hypoglycemia decreases leptin levels. We will measure leptin levels at baseline and then again two days following exposure to three bouts of insulin-induced hypoglycemia. Without wishing to be bound by theory, exposure to recurrent hypoglycemia will lead to a >50% reduction in leptin levels.

Aim 2: Establish whether leptin levels predicts susceptibility to hypoglycemia. We will measure leptin levels just prior to exposure to insulin-induced hypoglycemia and determine how well this measurement predicts the magnitude of the counterregulatory responses to the subsequent bout of hypoglycemia. Without wishing to be bound by theory, individuals will low leptin levels will be more susceptible to hypoglycemia, that is, as leptin levels decrease, the neuroendocrine response will also decrease.

Objectives

We will conduct a prospective observational study in healthy men and women to validate that exposure to recurrent hypoglycemia leads to hypoleptinemia. We will employ a recurrent hypoglycemia paradigm that has been previously validated to induce HAAF in healthy adults. We will measure subjects' leptin levels at baseline and throughout the recurrent hypoglycemia paradigm. Without wishing to be bound by theory, exposure to recurrent hypoglycemia will significantly reduce leptin levels.

Study Subjects

We will enroll up to 10 healthy men or women (goal n=8 completers) in this study.

Inclusion and Exclusion Criteria

Inclusion Criteria:

Ages 18-40 years

BMI between 20 kg/m² and 27.9 kg/m² (±0.5 kg/m² will be accepted)

Medically cleared for participation in the study

Willing to participate in continuous glucose monitoring (CGM)

### Exclusion Criteria:

History of clinically diagnosed diabetes or a fasting blood glucose >126 mg/dL

Average screening blood pressure >140/90 mmHg

History of cardiovascular disease

Use of medications affecting glucose metabolism, e.g., benzodiazepines, thiazide diuretics, cortisone, and prednisone.

Use of beta-adrenergic antagonists.

Pregnant, planning to become pregnant, or breastfeeding

Based on the investigative team's clinical judgement, a subject may not be appropriate for participation in the study.

If eligible, individuals will be enrolled and undergo all testing procedures.

Recruitment Methods

Subjects will be recruited through PBRC via IRB approved recruitment materials (e.g., landing page). Individuals can either complete the webscreening form directly from the PBRC landing page, call PBRC directly, or e-mail the Recruitment Core. Eligible subjects will then undergo a phone screen to answer a series of yes or no questions regarding eligibility. Eligible individuals will be scheduled for a screening visit at PBRC.

Study Timelines

A subject's duration of study participation will be approximately 8 days. The estimated duration to enroll all study subjects is anticipated to be 6 months. The estimated duration to complete the study is 9 months.

### Study Endpoints

Primary endpoints:
50% reduction in fasting leptin levels.
   Secondary endpoints:
Reduction in hypoglycemia-induced levels of serum epinephrine, norepinephrine, glucagon, and cortisol.
Change in CGM glucose

Procedures

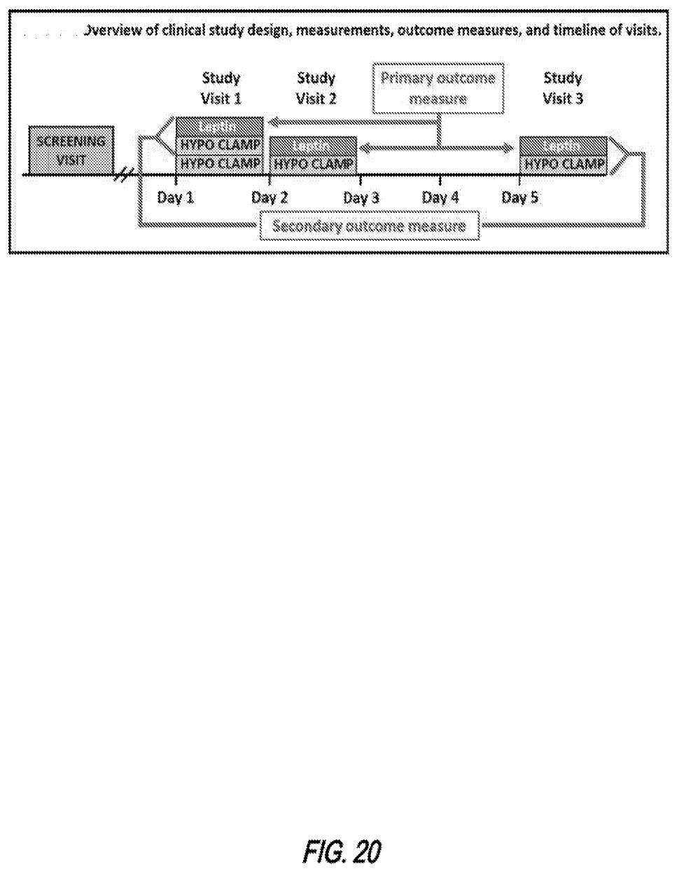

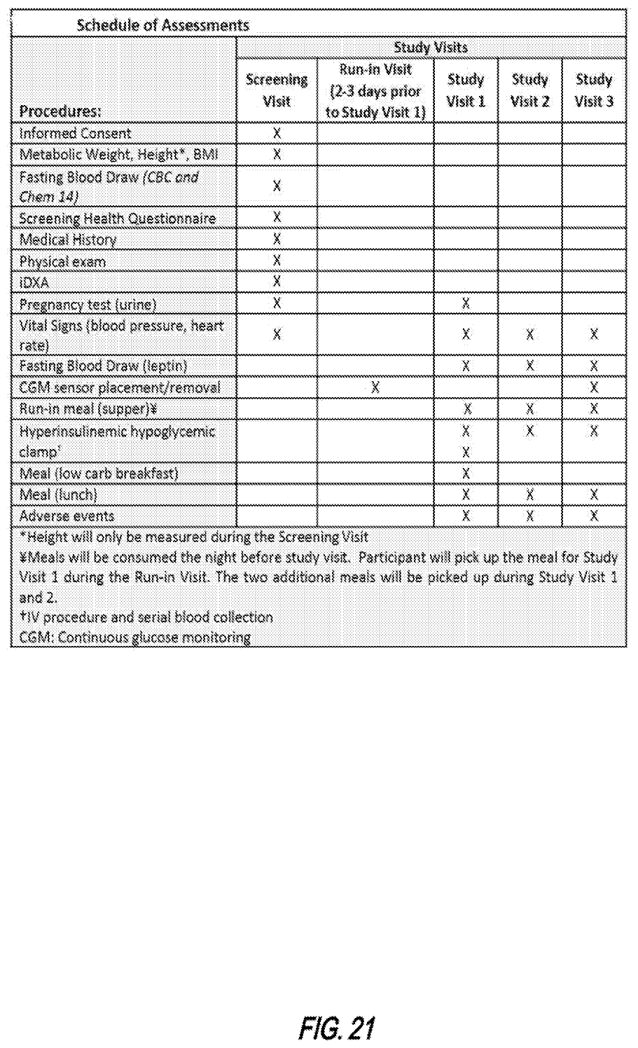

Subjects will complete a screening visit, a run-in visit, and three study visits conducted in the PBRC Inpatient Unit. See FIG. 20 for study design and FIG. 21 for schedule of assessments.

### Screening Visit:

Subjects will complete a screening visit at the Pennington Biomedical Outpatient Clinic to assess eligibility. Subjects will arrive in the morning to the PBRC Outpatient Unit, and after providing written informed consent, the following procedures will be completed: anthropometrics, vital signs, body composition (iDXA), fasting blood draw (CBC and Chem 14), screening heath questionnaire, and medical history and physical examination by one of the medical staff members. The study MI, Dr. Hsia, will conduct a final review of the subjects' charts to determine eligibility.

Study Visits:

Eligible subjects will be scheduled for the four study visits, which will occur over an approximately five-day period. Study Visit 1 and Study Visit 2 will be separated by 24 hours, while Study Visit 2 and Study Visit 3 will be separated by 72 hours.

Run-in Visit:

2-3 days prior to the Study Visit 1, subjects will be scheduled for a run-in visit to pick up their meal for Study Visit 1 and a continuous glucose monitor (CGM) sensor will be placed on their abdomen.

Study Visit 1:

Subjects will be provided a run-in meal (supper) which will be consumed the evening before their visit. Subjects will be instructed to 1) eat nothing but the meal provided

Subjects will arrive at the inpatient unit in the morning after at least a 10 hour overnight fast. Following anthropometrics, and pregnancy test, and blood draws, subjects will undergo a 2-hour hyperinsulinemic-hypoglycemic clamp (see below for details). Following completion of the clamp procedure, subjects will be fed a low carbohydrate breakfast (<10 g of carbohydrate) and blood glucose will be maintained within the range of 80-100 mg/dL for two hours via a 20% dextrose IV for 2 hours. Following this 2-hour interval, subjects will undergo a second 3-hour hyperinsulinemic-hypoglycemic clamp procedure. Following completion of the 2nd clamp, subjects will be fed lunch (Standard American Diet) and then released from the inpatient unit. Subjects will pick up their run-in meal for Study Visit 2 at this visit.

Study Visit 2 (1 day following Study Visit 1):

Subjects will be provided a run-in meal (supper) which will be consumed the evening before their visit. Subjects will be instructed to 1) eat nothing but the meal provided after 3 PM, 2) eat the entire meal before 9 PM, and 3) drink only water after 3 PM.

Following anthropometrics and fasting blood draw, participants will undergo a 3-hour hypoglycemic clamp. Lunch will be provided at the conclusion of the clamp and the participant will be discharged from the Inpatient Clinic. Subjects will pick up their run-in meal for Study Visit 3 at this visit.

Study Visit 3 (3 days after Study Visit 2):

Subjects will be provided a run-in meal (supper) which will be consumed the evening before their visit. Subjects will be instructed to 1) eat nothing but the meal provided after 3 PM, 2) eat the entire meal before 9 PM, and 3) drink only water after 3 PM.

Following anthropometrics and blood draws, participants will undergo a two-hour euglycemic clamp. Following completion of the clamp procedure, participants will be provided breakfast. The participant's CGM sensor will be removed following lunch and participants will be discharged.

Study Procedure Descriptions

Anthropometrics: Fasting body metabolic weight will be collected with subjects wearing a hospital gown and underwear. Height will be collected once at screening.

Blood collection (study use): Approximately 10 mL of whole blood will be collected during the screening visit. Approximately 390 mL of whole blood will be collected during the four hypoglycemic clamp procedures for measurement of glucose, potassium, and counter-regulatory hormones. The total volume of blood that will be collected during the study is approximately 400 mL.

Continuous glucose monitoring: Blood glucose will be assessed using continuous glucose monitoring (CGM). Briefly, the abdominal area will be disinfected, and then trained staff from the Inpatient Unit will insert a glucose sensor under the skin in the abdominal area. The sensor has a small needle-like probe that inserts into the subcutaneous fat of the abdomen and measures blood glucose levels without removing blood from the body. The sensor will then be attached to the recording unit, and the set-up will be secured with adhesive to the subject's body. After an initial period of equilibration with interstitial glucose, the sensor will be calibrated via the blood glucose measurement obtained during the hypoglycemic clamp procedure. The CGM device records interstitial glucose every 5 minutes and will allow a more complete profile of blood glucose changes during the study visit.

Hyperinsulinemic-hypoglycemic clamp: An intravenous catheter will be placed in an antecubital vein for infusion of insulin and glucose. A second catheter will be placed retrograde in a dorsal vein of the contra-lateral hand for blood withdrawal. The hand will be placed in a heating box or pad at 70° C. for arterialization of venous blood. A primed infusion of regular insulin (120 mU/min/m²) will be initiated and continued for approximately 2 hours. Beginning 20 minutes prior to the start of the insulin infusion, arterialized venous blood glucose will be measured at 5-minute intervals via a Hemocue or YSI analyzer. Following initiation of insulin infusion, blood glucose will be allowed to fall to 50 mg/dL and then maintained at this level using a variable infusion of exogenous dextrose (20% solution). Our goal is to achieve steady-state (blood glucose stabilized at 50+/−5 mg/dL) within the first 45 minutes following the start of insulin infusion, thus subjects blood glucose will be maintained at this level for approximately 75 minutes.

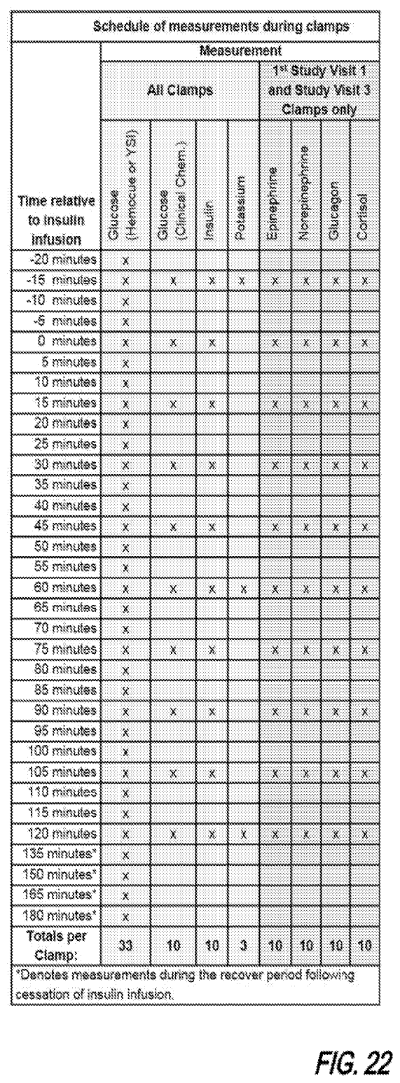

Potassium levels will be measured at three of these time points: −15, 60, and 120 minutes (relative to the start of the insulin infusion). See FIG. 22 for an overview of the timing of blood draws and measurements.

Following discontinuation of the insulin, blood glucose will be normalized to baseline levels with the 20% dextrose infusion. The subject's blood glucose will continue to be monitored every 15 minutes via Hemocue or YSI analyzer for approximately 60 minutes after discontinuation of insulin infusion to ensure baseline levels of blood glucose are achieved and maintained.

If potassium levels are below 3.8 mmol/1 at any of the three measurements (−15, 60, or 120 minutes) a potassium infusion of 10 mEq/hr will be initiated and continued for the remainder of the procedure. In the event that a potassium infusion is initiated, potassium levels may be measured again following discontinuation of insulin infusion, i.e. during the 60-minute recovery period when blood glucose levels will be restored and maintained at baseline levels (120-180 minutes relative to the start of the insulin infusion).

During the first clamp of Study Visit 1 and the Study Visit 3 clamp, additional blood will be collected for measurement of the counterregulatory hormones: glucagon, epinephrine, norepinephrine, and cortisol. These collections will occur every 15 minutes starting 15 minutes prior to initiation of the insulin infusion. See FIG. 22 for an overview of the timing of blood draws and measurements.

Dual-Energy X-ray Absorptiometry (DXA):

The DXA scans will be performed at the Imaging Facility of PBRC at Visit 1. Total adiposity and regional fat mass will be assessed with DXA using a whole-body scanner (Lunar iDXA; General Electric, Milwaukee, Wis.). The DXA protocol requires that subjects lie on a table wearing a hospital gown and with no metal objects on them while both legs will be placed together using two Velcro straps. The scanner emitting low energy X-rays, and a detector passes along the body. The scan takes ˜10 minutes and the radiation dose is less than 1 mrem, equal to about 12-h of background radiation. The scans will be analyzed with the latest software. The software version is enCORE 13.4. We will run quality control scans on a daily basis, and GE has indicated that accuracy of the data is confirmed with these daily QC scans. A pregnancy test (urine) will be given before DXA scans to confirm absence of pregnancy.

Questionnaire: Subjects will complete a Screening Health Questionnaire (to assess general health) at the screening visit.

Vital Signs: Vital signs will be collected according to PBRC standard operating procedures. Seated vital signs (blood pressure and heart rate) will be measured after a 5-minute rest.

Setting

This study will be conducted at Pennington Biomedical Research Center in the Outpatient Unit and Inpatient Unit.

Compensation

Subjects will receive up to $450 upon completion of the study. If a participant does not complete the entire study, they will be compensated $200 for Visit 1, $125 for Visit 2, and $125 for Visit 3. No compensation will be provided for the screening or run-in visit.

Data and Specimen Management

Study data collected and entered into the Pennington Biomedical Database is handled only by individuals with appropriate HIPAA compliance and Good Clinical Practice training. Subject charts and hard copy data are stored in locked offices with restricted access. Electronic data has exclusive restricted access granted by the Research Computing Group and/or the PI. For quality control, data and charts will be audited for completeness and accuracy.

Provisions to Monitor the Data to Ensure the Safety of Subjects

We will use the definitions of Adverse Events, Serious Adverse Events, and Unanticipated Problems Involving Risks to Subjects or Others below. Events will be recorded from the subject during their inpatient stay by experienced staff trained in the ascertainment of adverse events from research subjects. For each sign, symptom or adverse event, the following information will be recorded:

A brief descriptor of the adverse event

Date of onset and date of resolution

Frequency (single/intermittent)

Maximum intensity (mild/moderate/severe)

Outcome (resolved/resolved with sequelae/not resolved)

Action taken with respect to study drug/intervention (none/dose reduced/temporarily interrupted/permanently discontinued/intervention).

Withdrawal (yes/no)

Relationship to study drug/intervention

Whether the AE was “serious” or not (as defined below)

The Pennington Biomedical Research Center's Human Research Protections Program's definitions for adverse event, serious adverse event, and unanticipated problem involving risks to subjects or others (Policy 8) will be applied in this study and are as follows:

An adverse event can refer to any untoward physical or psychological occurrence in a human subject participating in research, including any abnormal sign (e.g., abnormal physical exam or laboratory finding, symptoms or disease associated with the research or the use of a medical investigational test article), symptom, or disease, temporally associated with the subject's participation in the research. An adverse event does not necessarily have to have a causal relationship with the research, or any risk associated with the research or the research intervention, or the assessment.

A serious adverse event can be defined as an adverse event that is fatal or life-threatening, permanently disabling, requires or prolongs hospitalization or results in significant disability, congenital anomaly or birth defect.

An unanticipated problem involving risks to subjects or others can be defined as any incident, experience, outcome or new information where all three elements exist:
Is unexpected;
Is related to participation in the research, and
Indicates that subjects or others are at a greater risk of harm (including physical, psychological, economic, or social harm) than was previously known or recognized.
   While federal guidelines do not require the reporting of adverse events to the IRB, unanticipated problems involving risks to subjects or others will be reported to the IRB within 10 working days of ascertainment of the event per HRPP guidelines.

Upon completion of each subject, subject's anthropometric and adverse event data will be reviewed by the Medical Investigator with the Principal Investigator to evaluate the data collected regarding both harms and benefits to determine whether subjects remain safe. When incidental findings on imaging studies or out of range values on lab tests are obtained by study personnel, the subject will be notified and a copy of the report sent to his physician. For lab tests, this pertains only to those tests for which results are obtained in real-time.

### Example 6

Leptin treatment prevents impaired hypoglycemic counter-regulation induced by exposure to recurrent hypoglycemia or caloric restriction.

Background: Hypoglycemia-associated autonomic failure (HAAF) is a significant barrier to achieving optimal glucose levels in persons with diabetes. It has been demonstrated that low leptin levels are concurrent with impaired hypoglycemic counter-regulation in two separate animal models of HAAF: six days of 60% caloric restriction (CR) in mice and 3 days of recurrent hypoglycemia (3dRH) in rats. In this study, we tested whether leptin treatment during CR or 3dRH could prevent the development of HAAF.

Methods: In experiment one, 3 groups of mice (two CR groups and one ad-lib fed control group) received either twice daily leptin (0.5-1 μg/g, IP) or PBS. In experiment two, 3 groups of rats (two 3dRH and one control) received daily leptin or PBS injections (0.3-0.7 mg/kg, IP). One day following the CR or 3dRH paradigms, all animals underwent a hypoglycemic ITT and levels of glucagon, leptin, epinephrine (rats), and corticosterone were assessed 60 min following insulin treatment.

Results: PBS treated CR mice and 3dRH rats had significantly reduced glucagon levels and epinephrine levels relative to controls (24±5 vs. 95±20 pmol/L and 57±12 vs 110±9 ng/mL, respectively). In contrast, there was no differences in the glucagon levels of leptin treated CR mice, and epinephrine levels of leptin treated 3dRH rats relative to their respective control groups. Leptin levels in both CR groups were reduced relative to controls (0.9±0.3 and 0.2±0.1 vs. 3.0±0.5 ng/mL, respectively), while corticosterone was unchanged. Glucagon, corticosterone, and leptin levels were similar across groups in the 3dRH paradigm.

Discussion: Utilizing two separate animal models of HAAF, we established that leptin treatment can prevent the loss of hypoglycemic counter-regulation. Future studies investigating whether combining leptin treatment with insulin therapy could significantly reduce hypoglycemic complications in diabetes patients are warranted.

### Example 7

Leptin supplementation prevents the loss of hypoglycemia-induced glucagon release following exposure to six days of severe caloric restriction in mice.

Objectives/Goals:

We have recently shown that mice exposed to six days of 60% caloric restriction acutely display reduced hypoglycemia-induced glucagon release following refeeding, and that this effect is concurrent with low leptin levels. The current study was conducted to ascertain if leptin treatment during caloric restriction would reverse this effect.

Methods:

Three groups of mice were used, an ad libitum (Ad-lib) fed group and two caloric restriction (CR) groups, one of which received twice daily leptin injection (0.5-1 μg/g; IP) and the other vehicle (saline) during their caloric restriction. CR mice were placed on 60% caloric restriction for 6 consecutive days. Ad lib mice were housed in an identical manner but fed ad libitum during this same period. Following 6 days of restriction, CR mice were given ad lib access to food for 16 h. After the 16 h period of refeeding, both CR and ad lib mice began a 6 h fast which was immediately followed by a hypoglycemic insulin tolerance test (ITT). ITTs consisted of a variable dose of insulin intended to achieve a blood glucose of ˜45 mg/dL within 60 minutes, at which time blood was collected for glucagon and corticosterone assays.

Results:

The mean blood glucose levels during the ITT at 45- and 60-minutes post injection across all three groups were 46.8±3.1 and 37.0±2.4, respectively. There were no significant differences in glucose levels between the three groups at these two time points. Saline treated CR mice displayed significantly reduced serum glucagon levels in response to the ITT relative to Ad-lib mice (23.5±10.9 vs. 91.7±20.8 pg/mL, p=0.009). In contrast, leptin-treated CR mice maintained their hypoglycemia-induced glucagon response to the ITT (78.0±16.8 pg/mL, p>0.99 vs. Ad-lib group). In addition, although corticosterone levels in saline treated CR mice were numerically lower than in Ad-lib mice, this difference was not statistically significance (3928±277 vs. 4571±178 pg/mL, p=0.179).

Discussion/Significance of Impact:

Diabetes patients on insulin therapy often develop impaired hypoglycemic counter-regulation which can lead to life-threatening hypoglycemic complications. Without wishing to be bound by theory, our results indicate that leptin can be a therapeutic intervention for the prevention of impaired hypoglycemic counter-regulation in persons with diabetes.

### Example 8

Clinical Significance

Although diabetes is thought of primarily as a disease of hyperglycemia, treatments often lead to severe hypoglycemia, which makes management of proper glucose homeostasis extremely problematic. Diabetes is commonly thought of as a disease of high blood glucose or hyperglycemia. Yet the treatment of diabetes often leads to severe hypoglycemia or low blood glucose, and these hypoglycemic crises are a major impediment to the maintenance of healthy glucose levels in diabetes patients.

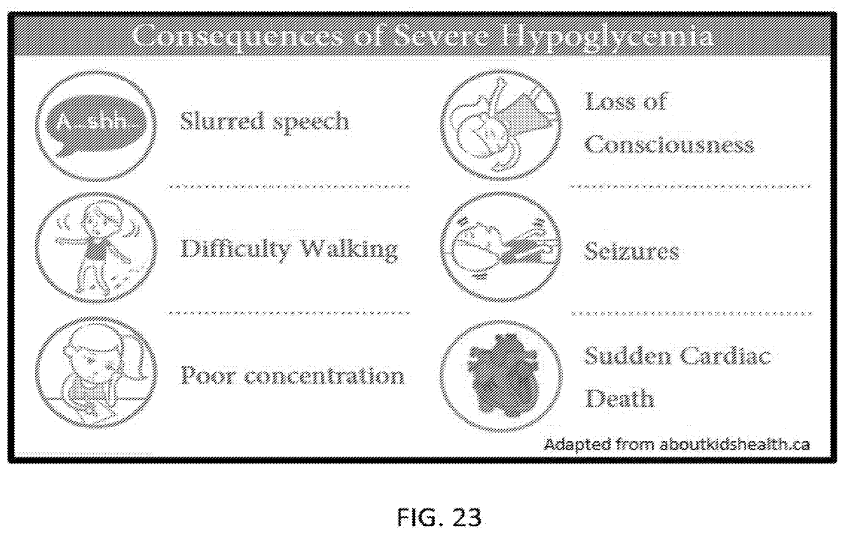

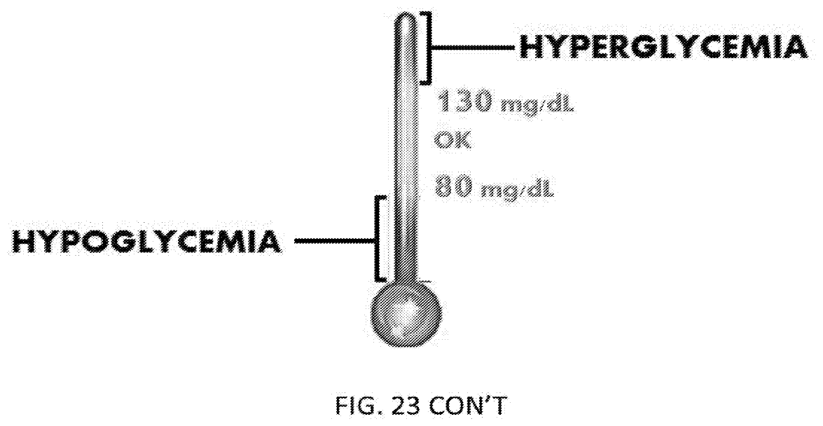

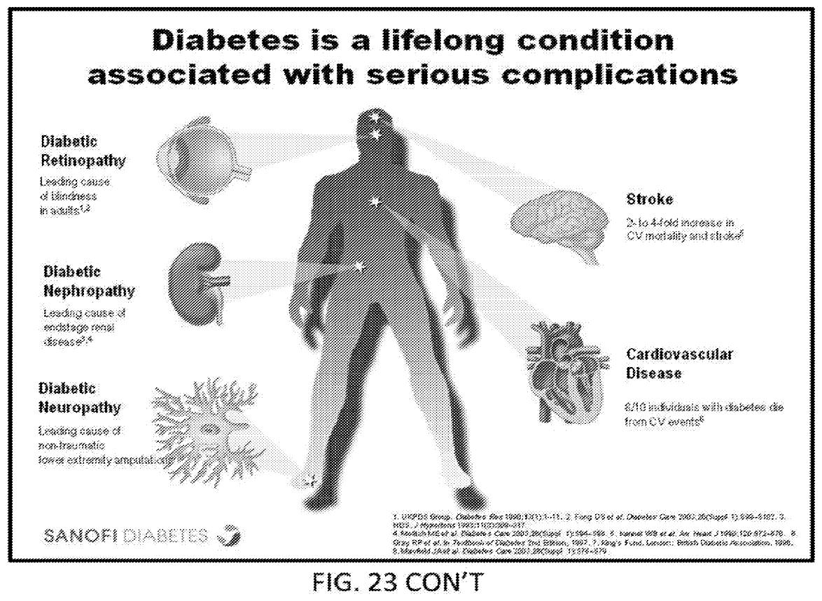

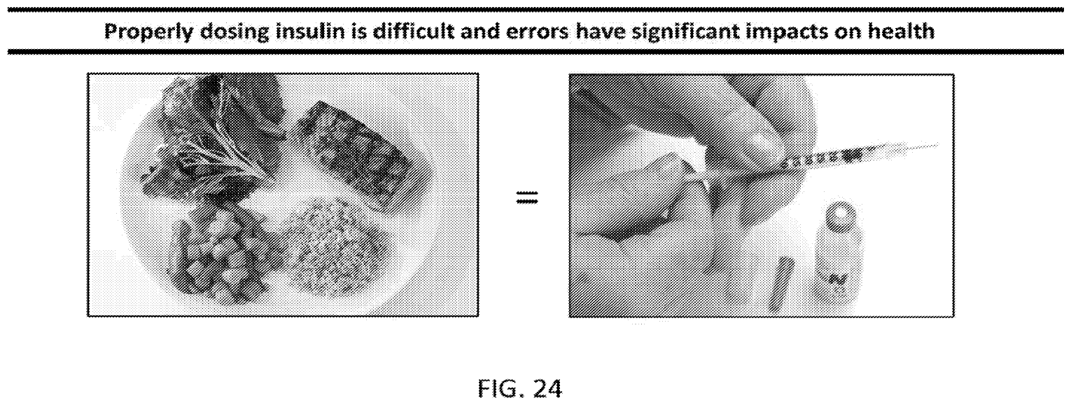

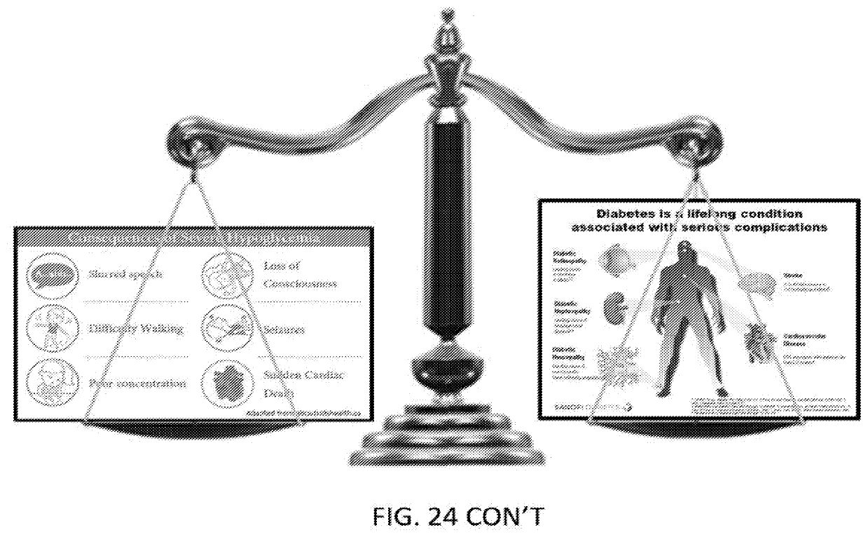

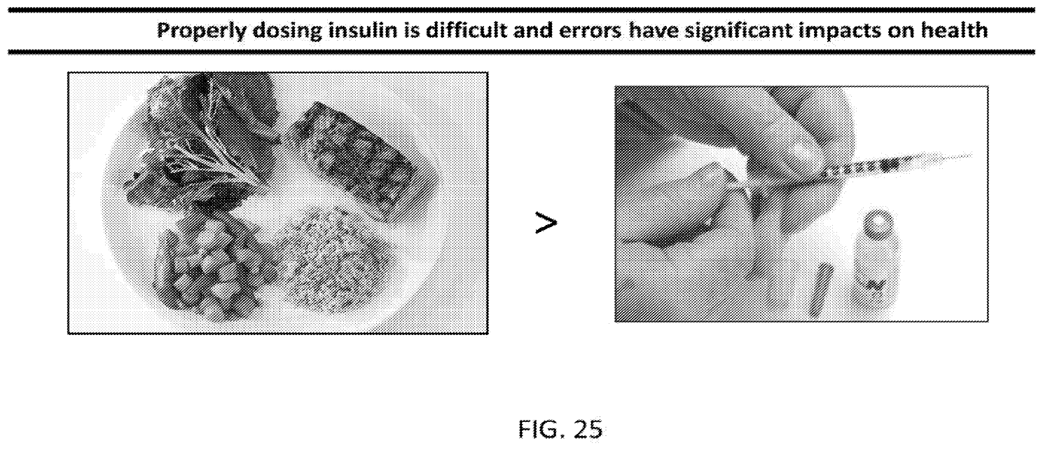

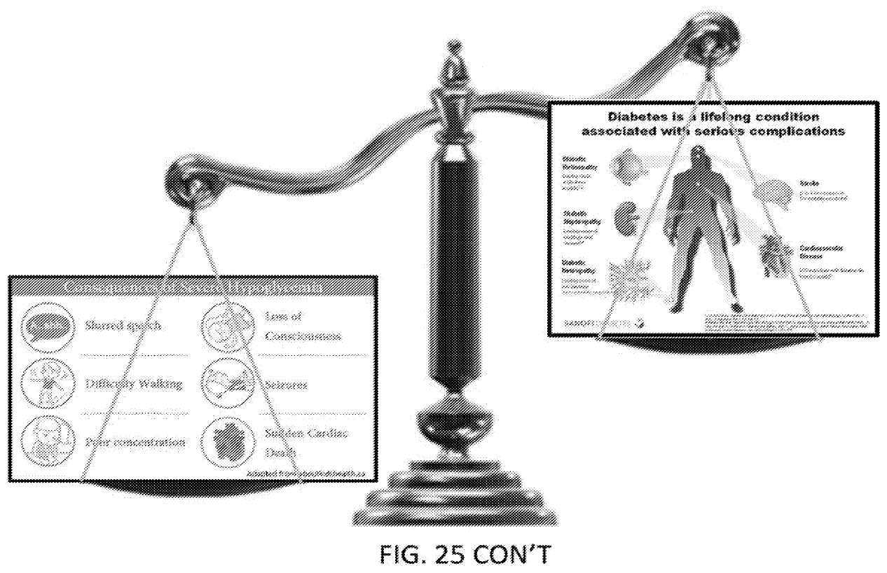

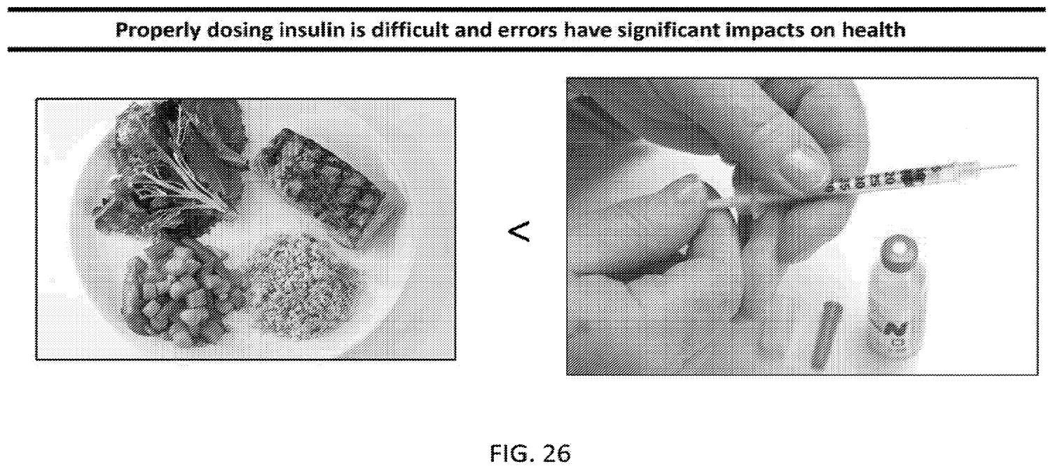

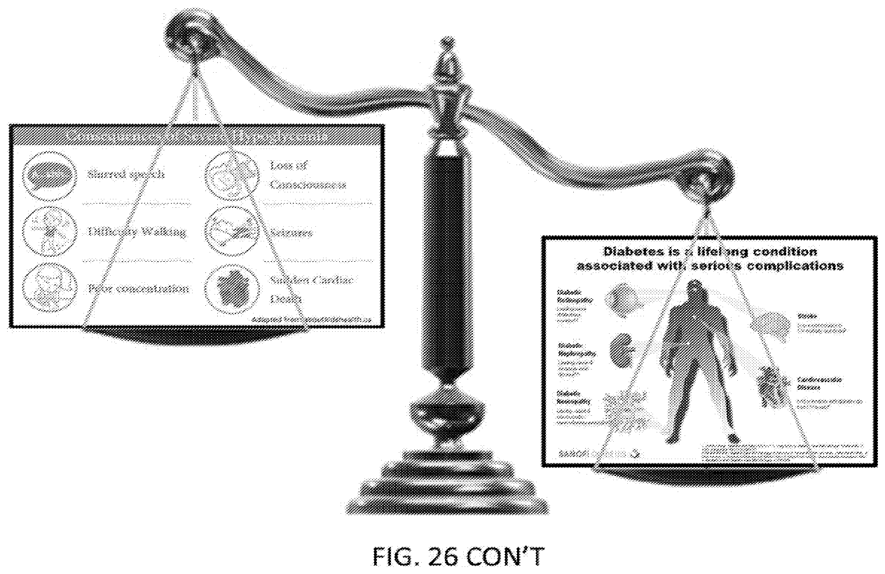

Current clinical recommendations indicate that diabetes patients maintain their blood glucose between 80 and 130 milligrams per deciliter (FIG. 23). Properly dosing insulin is difficult and errors have significant impacts on health. Insulin therapy is standard treatment for individuals with type 1 diabetes and is often required in patients with longstanding type 2 diabetes. Yet, properly dosing insulin is difficult and errors have significant impact on patient health. Therefore, individuals with diabetes face a tremendous treatment burden where they are required to balance the relative risks of hypoglycemia versus hyperglycemia multiples times per day. When they calculate their insulin dose perfectly, based on the nutrient content of their meals as well as their activity level, these risks are perfectly balanced (FIG. 24). If they administer too little insulin, their risk for long term complications such as blindness and kidney failure go up (FIG. 25). If they administer too much insulin, their risk for short term complications such as loss of consciousness and cardiac arrest goes up (FIG. 26). An aspect of the invention is to help relieve this treatment burden by working to find new treatment options which independently reduce the risk for hypoglycemia without raising the risks for the chronic complications associated with hyperglycemia.

Clinical Significance HAAF

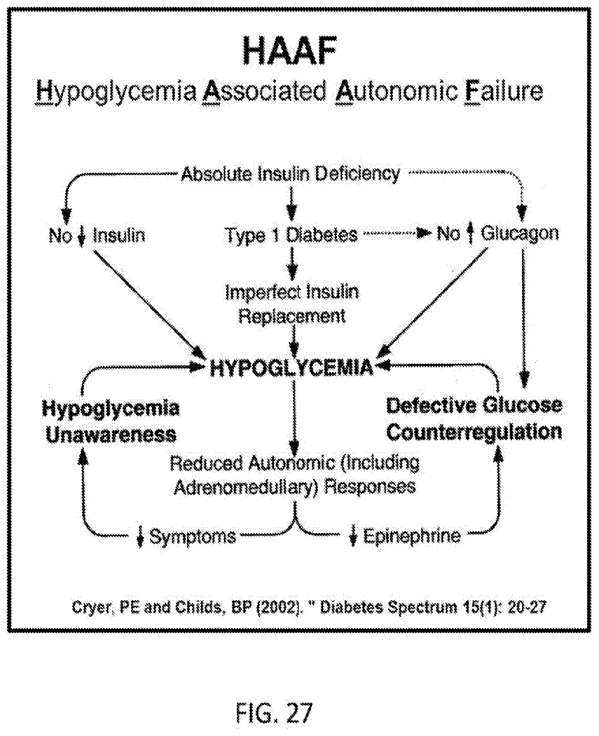

Recurrent bouts of insulin-induced hypoglycemia can lead to a serious clinical syndrome called HAAF (Hypoglycemia Associated Autonomic Failure) (FIG. 27). This condition is characterized by a complete lack of glucose counter-regulation which causes diabetic patients to be extremely vulnerable to life-threatening bouts of severe hypoglycemia. The hallmark of hypoglycemic complications is a condition called HAAF, or hypoglycemia associated failure.

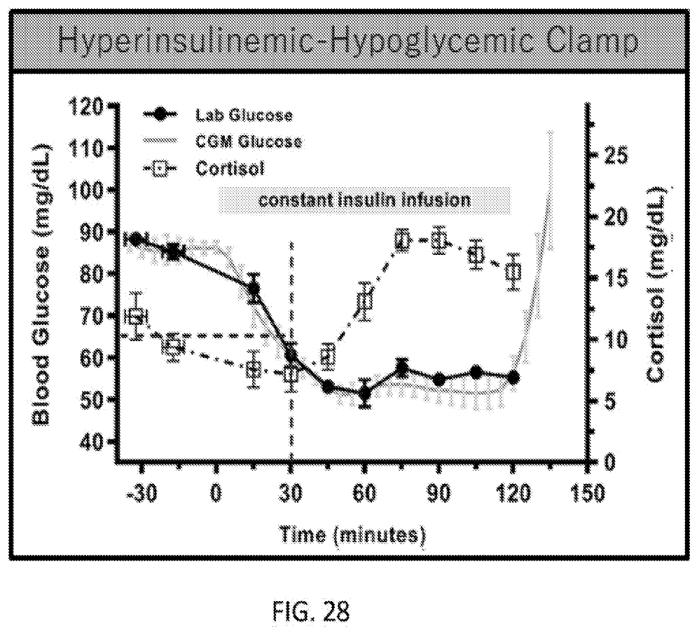

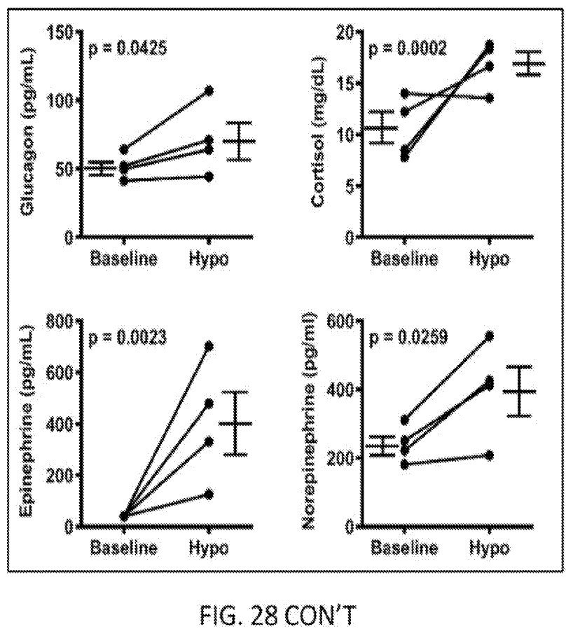

We can measure glucose counter-regulation with the hyperinsulinemic-hypoglycemic clamp (FIG. 28). This technique uses a paired infusion of glucose and insulin, to carefully control a subject's blood glucose. Once the infusions are started, blood glucose is allowed to fall to ˜50 mg/dL and maintained at this level for ˜2 hours, and then is rapidly returned to normal levels. This technique not only allows you to safely expose subjects to hypoglycemia, you can also quantify the individuals's physiological response to hypoglycemia. When blood glucose falls below around ˜65 mg/dL, a counter-regulatory response is engaged, which leads to the release of hormones, such as epinephrine, norepinephrine, cortisol, and glycagon, which act to counteract hypoglycemia. By measuring the levels of these hormones during the clamp, you get an indication of how robustly an individual respond to hypoglycemia.

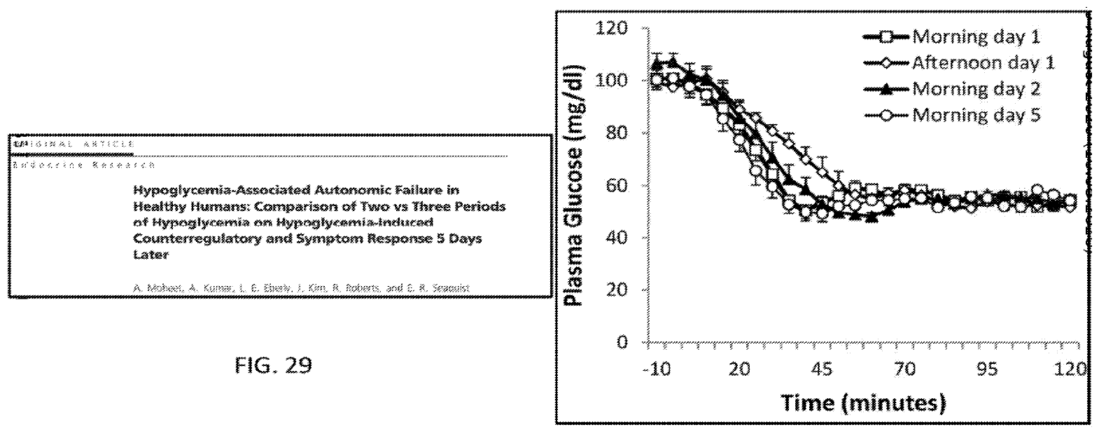

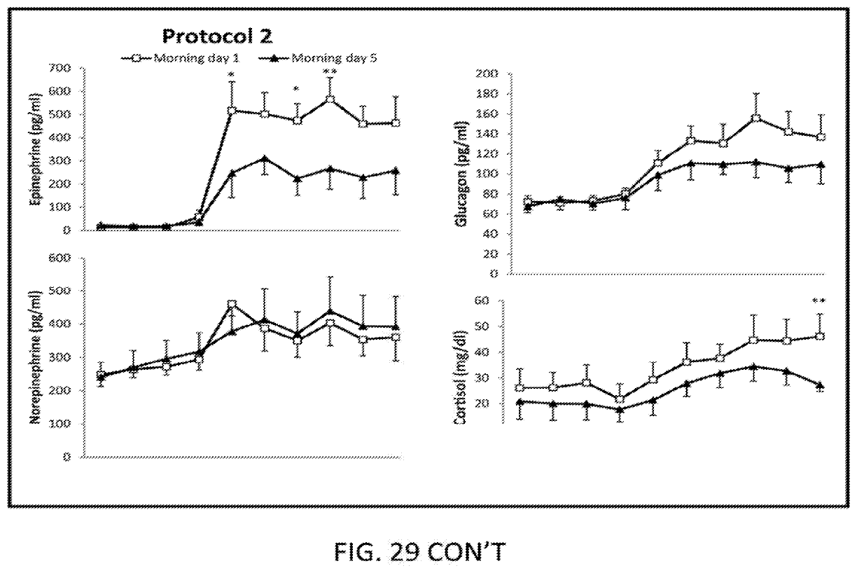

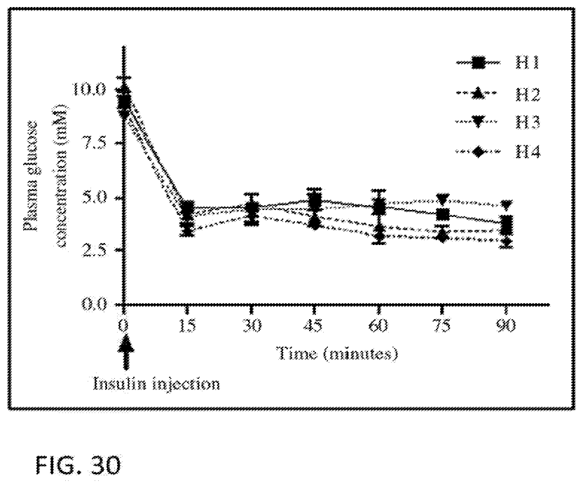

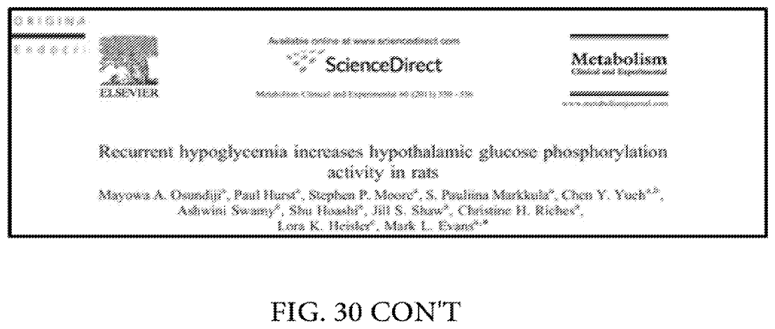

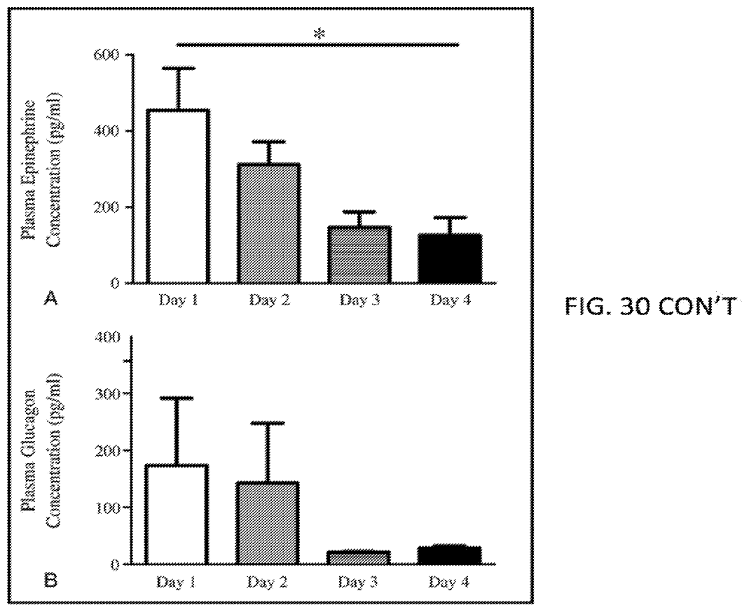

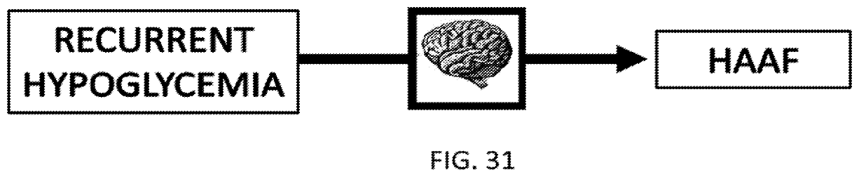

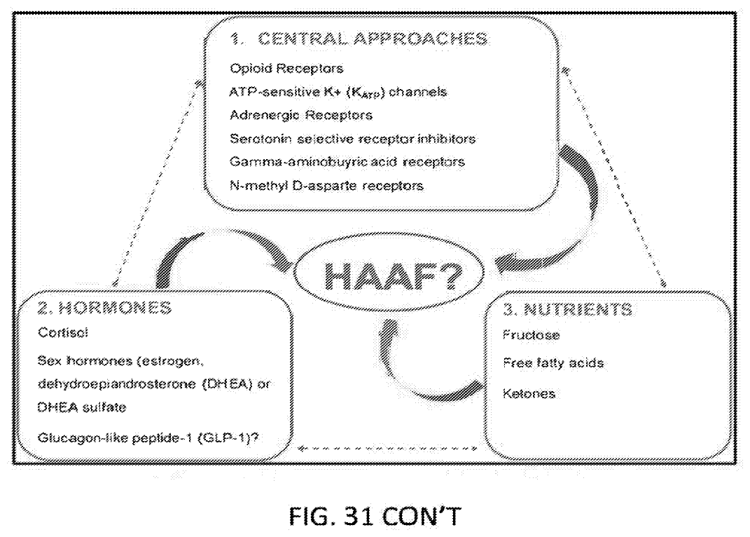

An interesting feature of HAAF, is that it's not dependent on the diabetes disease state, in other words a non-diabetic individual exposed to repeated bouts of hypoglycemia will develop HAAF. See, for example, FIG. 29. In this experiment, participants were exposed to 4 bouts of hypoglycemia over 5 days using the hypoglycemic clamp technique. This technique allows blood glucose to be safely lower to hypoglycemic levels while assessing the physiological response to the hypoglycemic challenges.

Glial Cells and HAAR Basic Science Approach

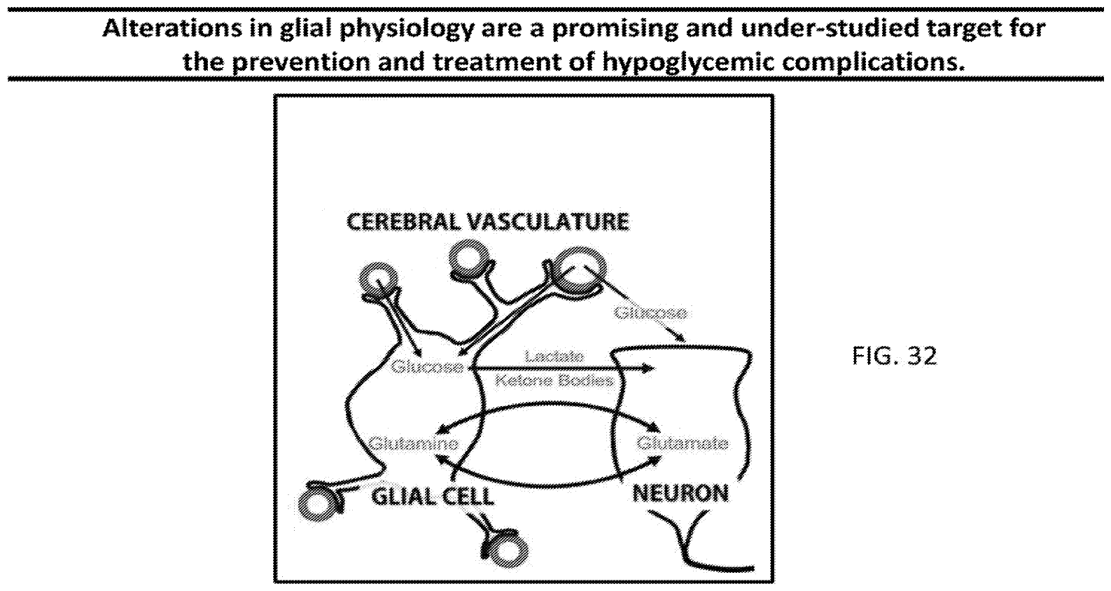

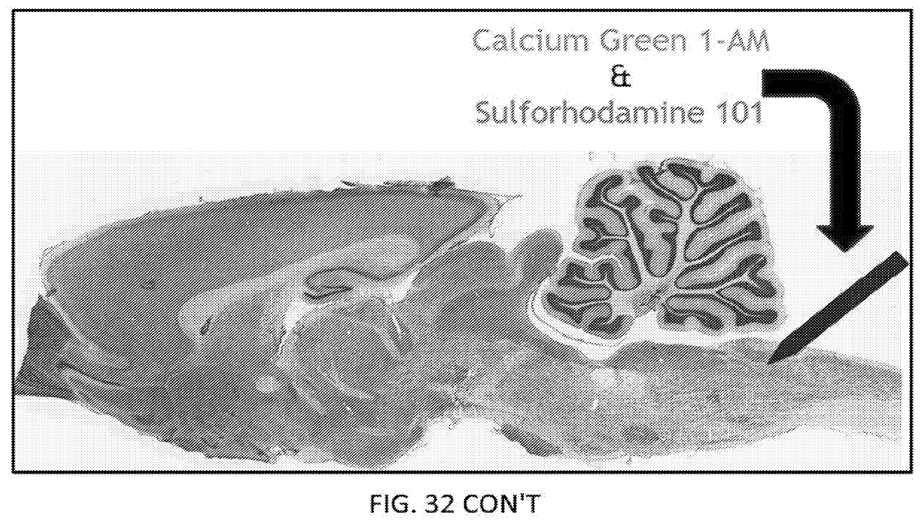

Alterations in glial physiology are a promising and under-studied target for ther prevention and treatment of hypoglycemic complications (FIG. 32). Cerebral metabolism is influenced by interactions between glial cells, neurons, and the cerebral vasculature.

Background

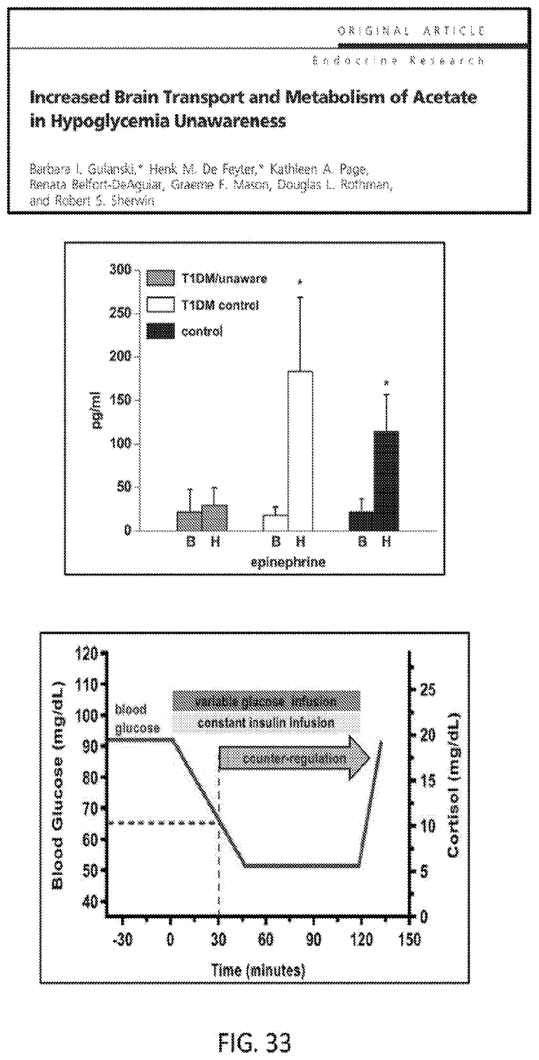

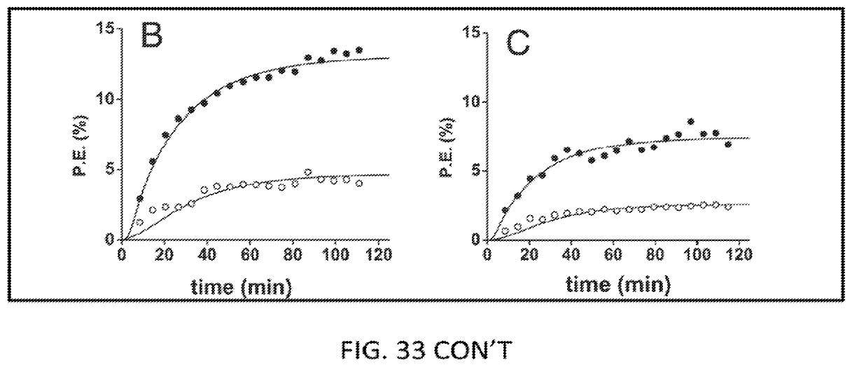

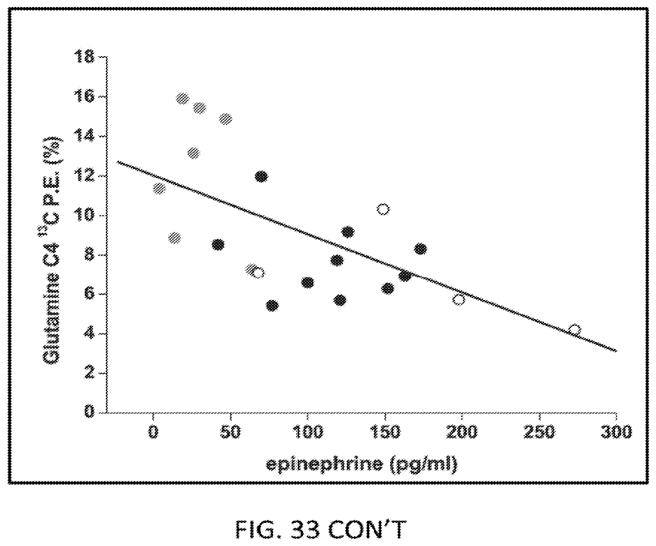

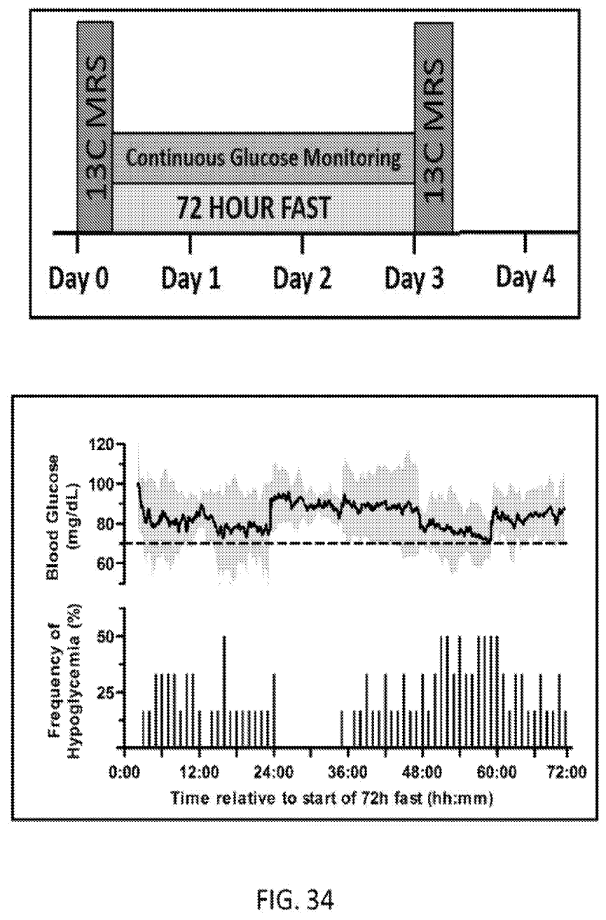

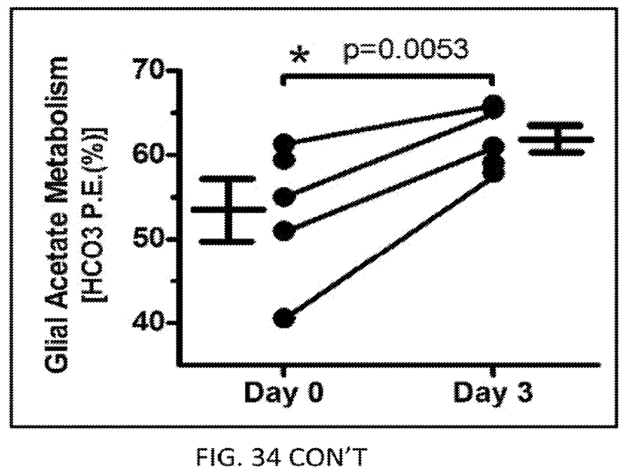

Cross-sectional evidence that glial acetate metabolism and the neuroendocrine response to hypoglycemia are closely associated when the two are measured simultaneously (FIG. 33). This study was the first interventional study to demonstrate that exposure to hypoglycemia leads to increases in glial acetate metabolism. The major findings of the study were that 72 hours of fasting leads to multiple transient bouts of hypoglycemia (FIG. 34) (McDougal et al. Acta diabetologica).

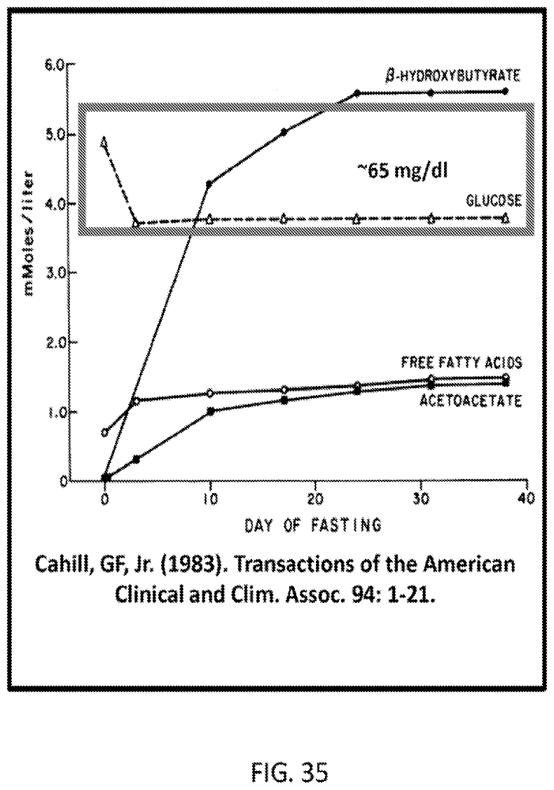

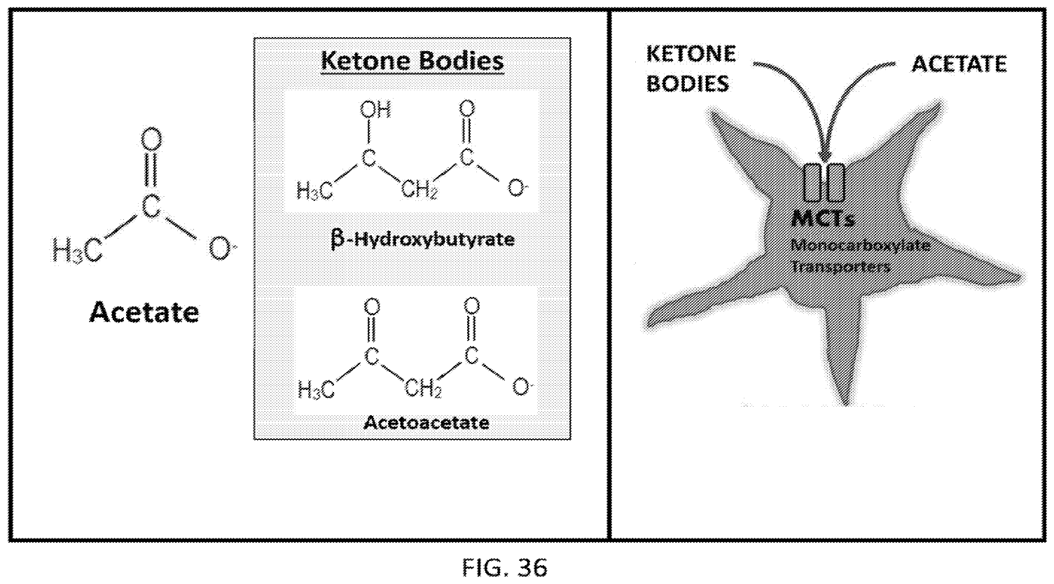

HAAF and Starvation
Starvation is associated with moderate to severe hypoglycemia. During starvation, blood glucose falls to ˜65 mg/dl within 2-3 days and then stabilizes (FIG. 35) (Cahill, G F, Jr. (1983) Transactions of the American Clinical and Climatalogical Association). Concurrently, blood ketones levels rise dramatically.
Starvation is associated with increased serum levels of ketone bodies, which are chemically similar to acetate (FIG. 36).

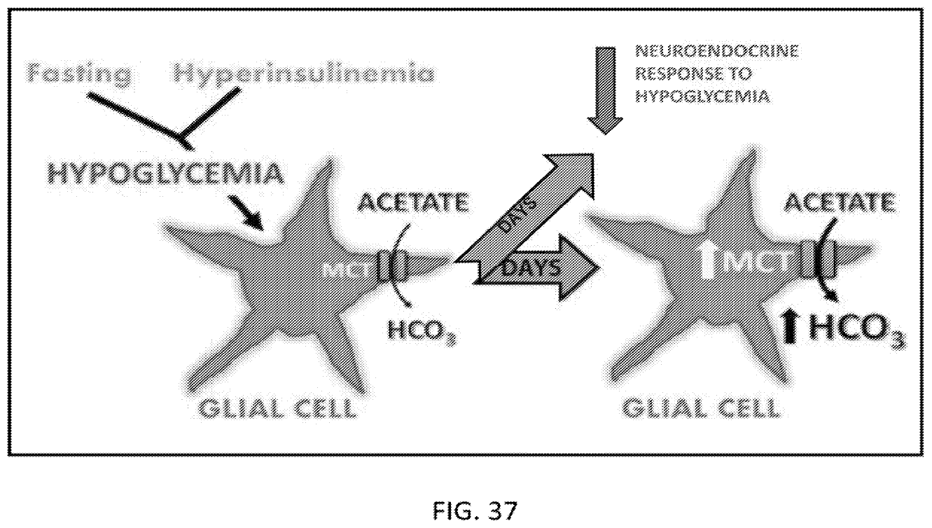

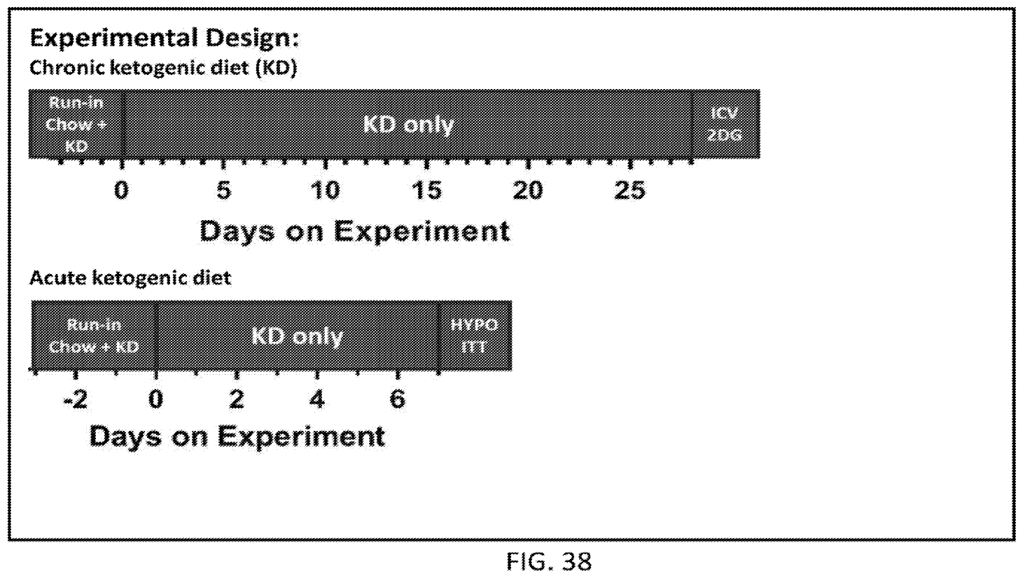

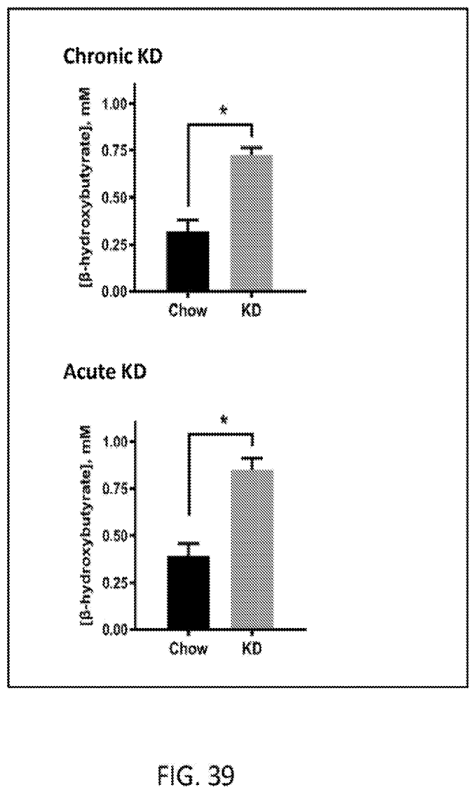

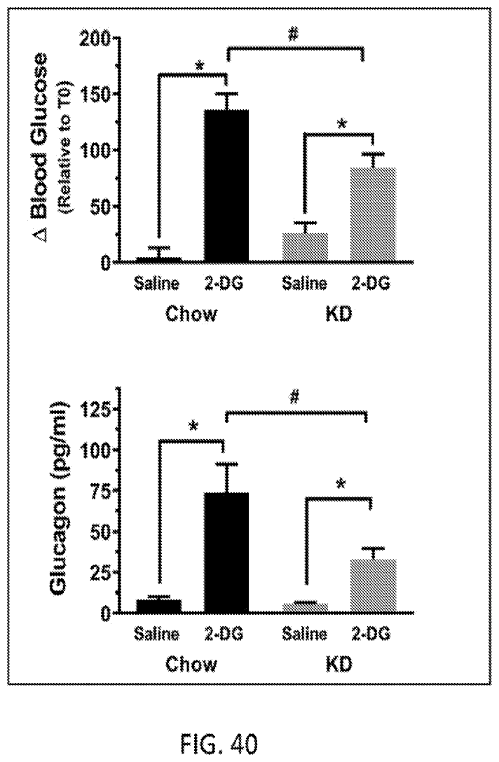

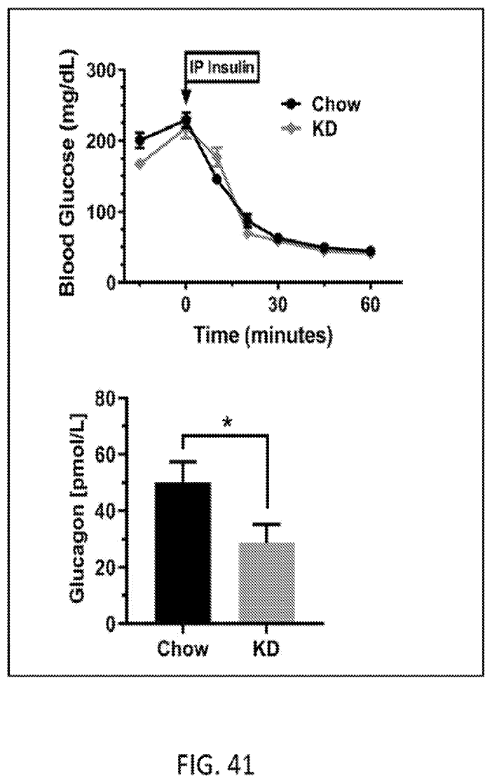

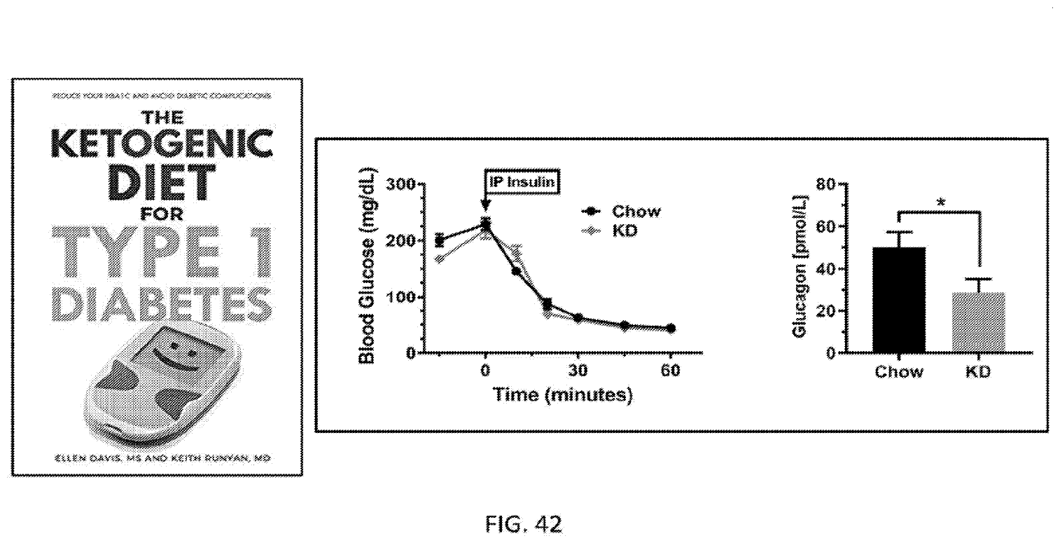

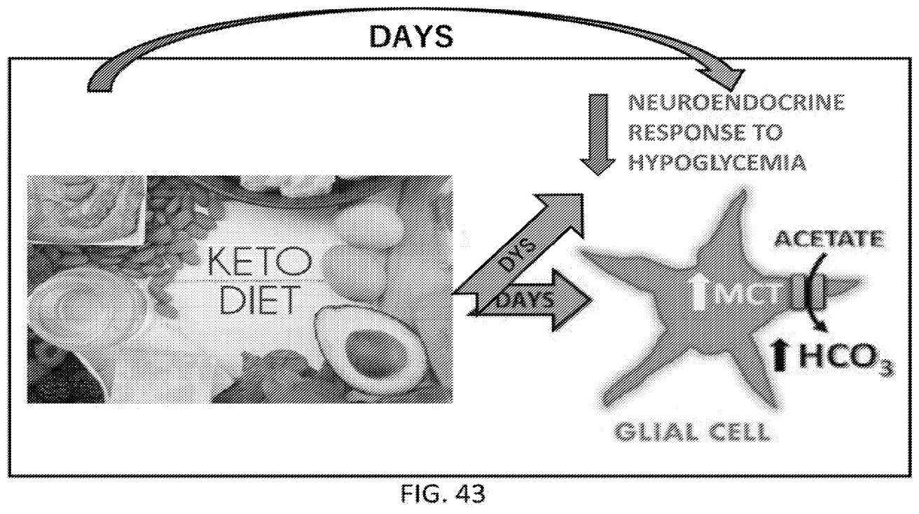

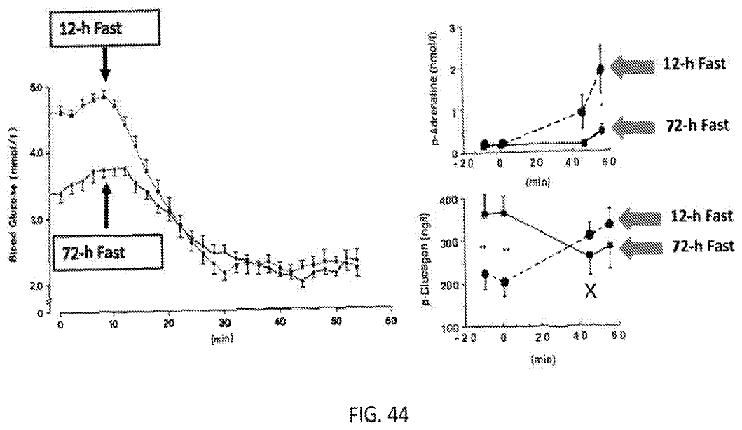

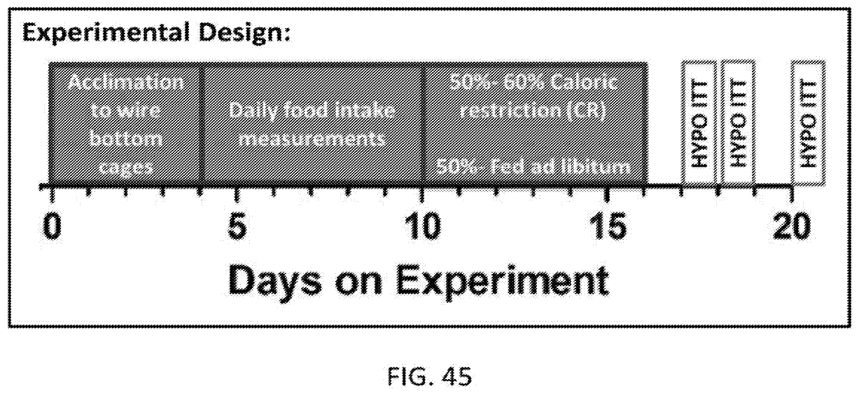

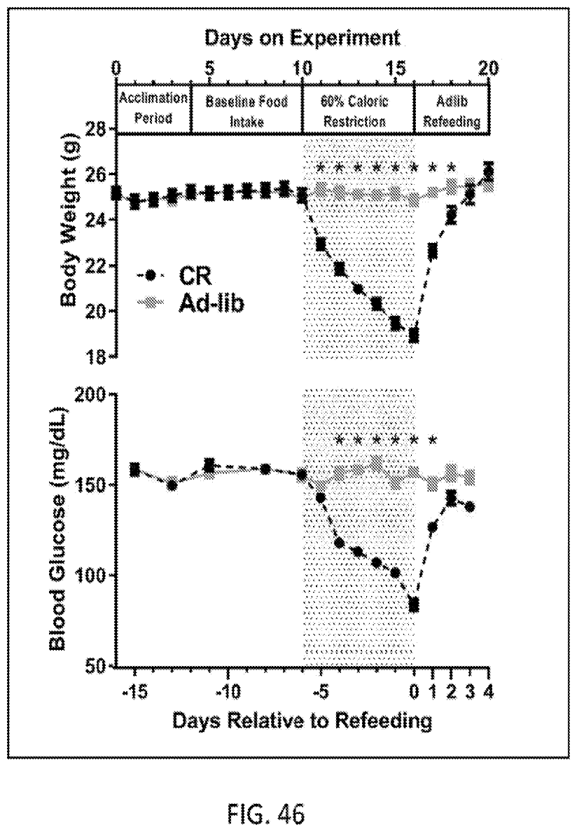

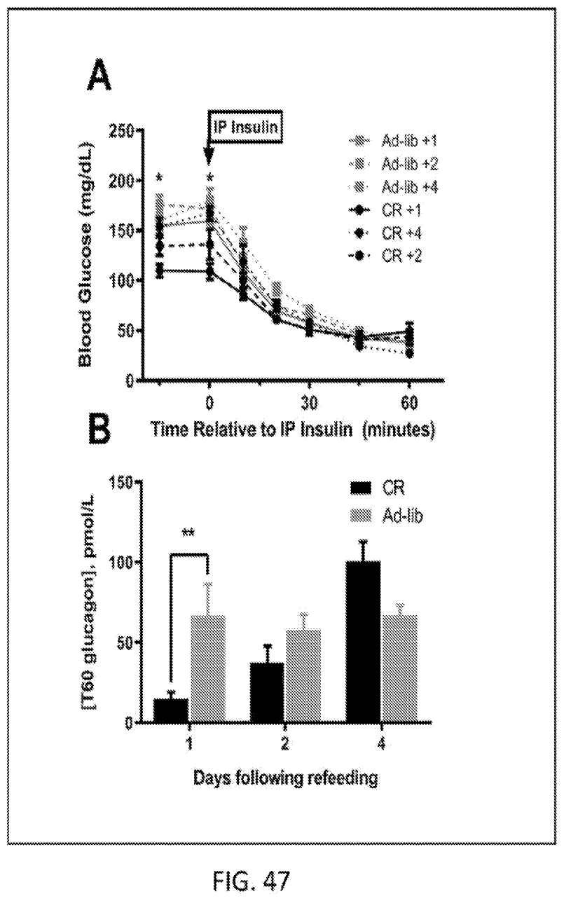

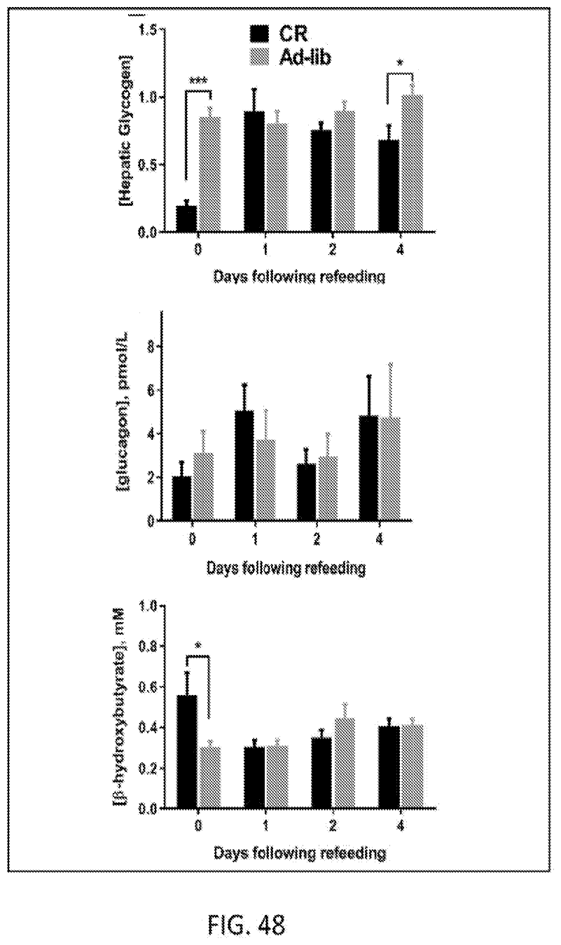

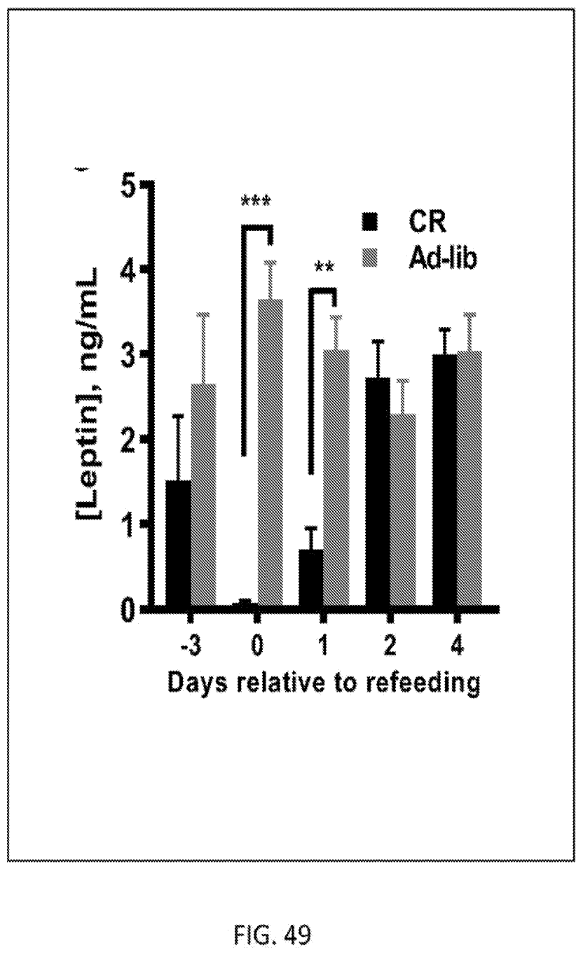

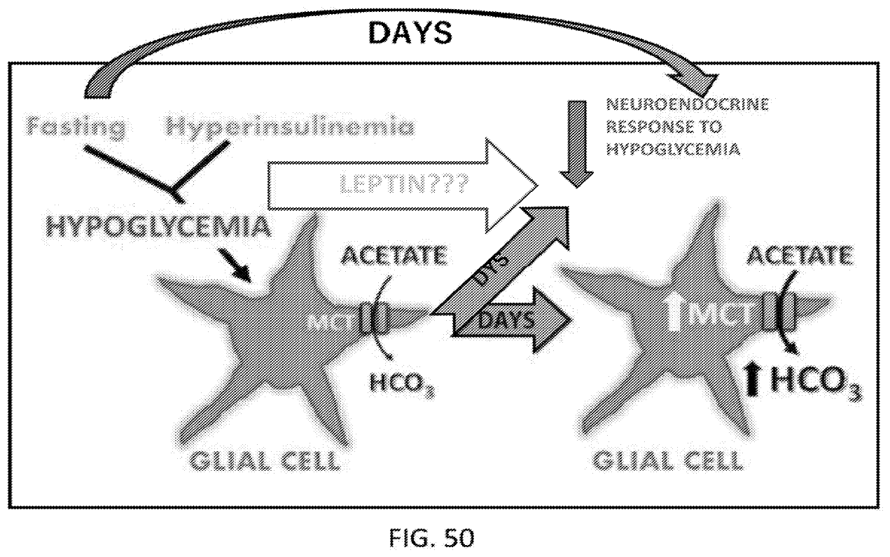

Without wishing to be bound by theory, HAAF develops as a consequence of metabolic adaptations to starvation which are pathophysiologically initiated due to exposure to insulin induced hypoglycemia (FIG. 37).
We want to know if exposure to a ketogenic diet leads to HAAF-like symptoms in mice (FIG. 38).
Mice fed a KD, either chronically or acutely, experienced ketosis. Mice exposed to either chronic or acute KD had significantly increased serum ketone concentrations (FIG. 39).
Chronic exposure to KD led to a blunting of the response to ICV 2-DG. Mice exposed to a chronic KD had significantly reduced responses to neuroglucopenia, indicated a reduction in 2-DF-induced elevations in glucose and glucagon (FIG. 40).
Mice acutely exposed to KD had impaired responses to hypoglycemia. Mice exposed to an acute KD experienced a significant reduction in glucagon release induced by insulin-induced hypoglycemia (FIG. 41).
Ketogenic diets are becoming increasingly popular as a secondary treatment for diabetes, including type 1 diabetes (FIG. 42).
Without wishing to be bound by theory, we explore if exposure to prolonged fasting leads to HAAF-like symptoms (FIG. 43).
72 hours of fasting leads to HAAF-like symptoms in metabolically healthy men (FIG. 44) (Adamson, U et al. Scandinavian journal of clinical and laboratory investigation 1989).
We want to know if prolonged exposure to starvation in mice via 60% caloric restriction, leads to HAAF-like symptoms in mice following refeeding (FIG. 45).
We found that 60% caloric restriction leads to reductions in body weight and blood glucose. Mice exposed to six days of 60% CR experienced a ˜24% drop in body weight and ˜45% drop in blood glucose (FIG. 46) (McDougal et al. Experimental Physiology).
Hypoglycemia-induced glucagon release was significantly impaired one day following refeeding (FIG. 47) (McDougal et al. Experimental Physiology).
Serum glucagon and ketones, as well as hepatic glycogen content rapidly normalized following refeeding (FIG. 48) (McDougal et al. Experimental Physiology).
Leptin levels did not normalize until two days following refeeding (FIG. 49) (McDougal, et al. Experimental Physiology).
Leptin signaling may be involved in the development of HAAF (FIG. 50).

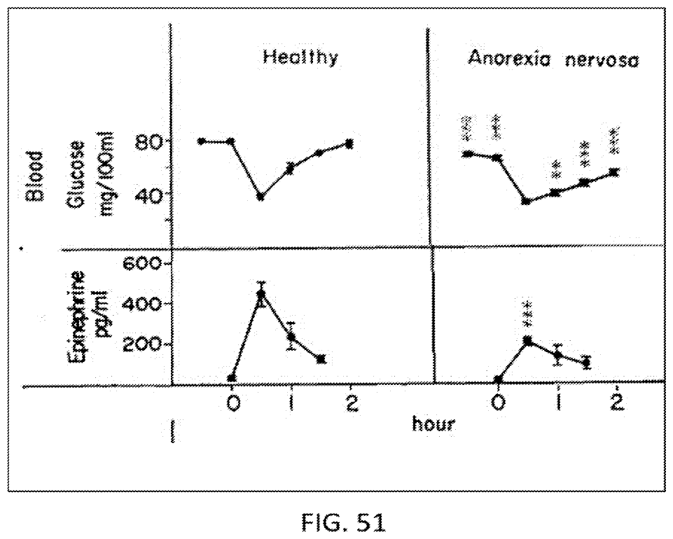

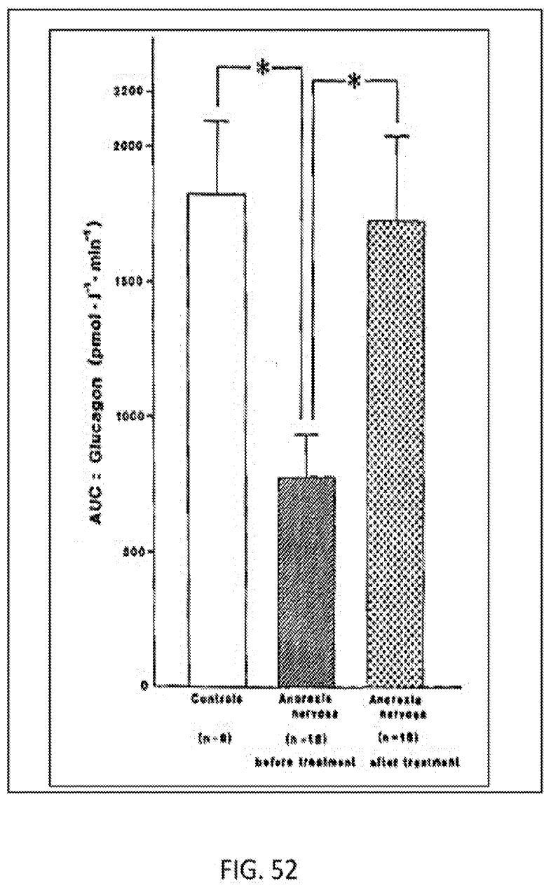

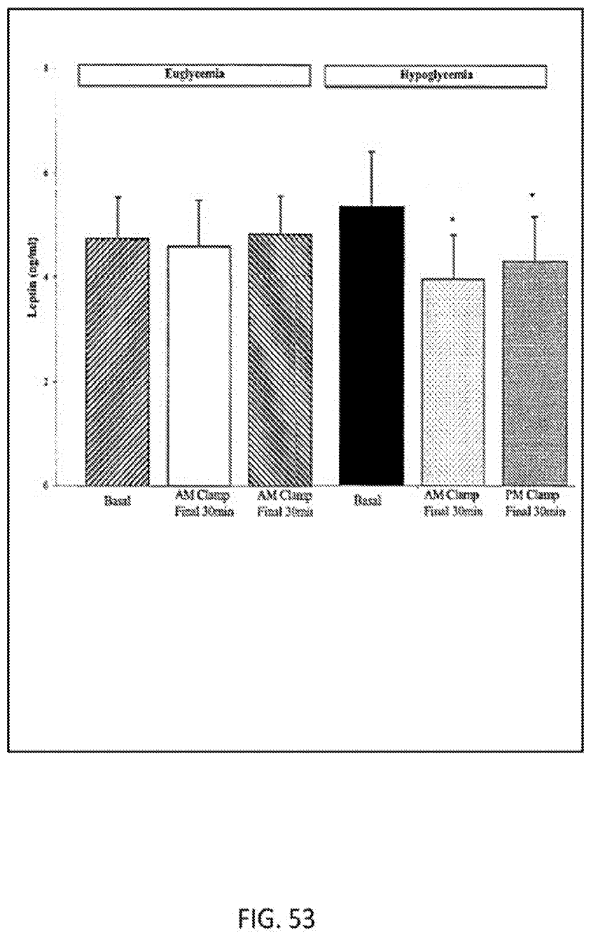

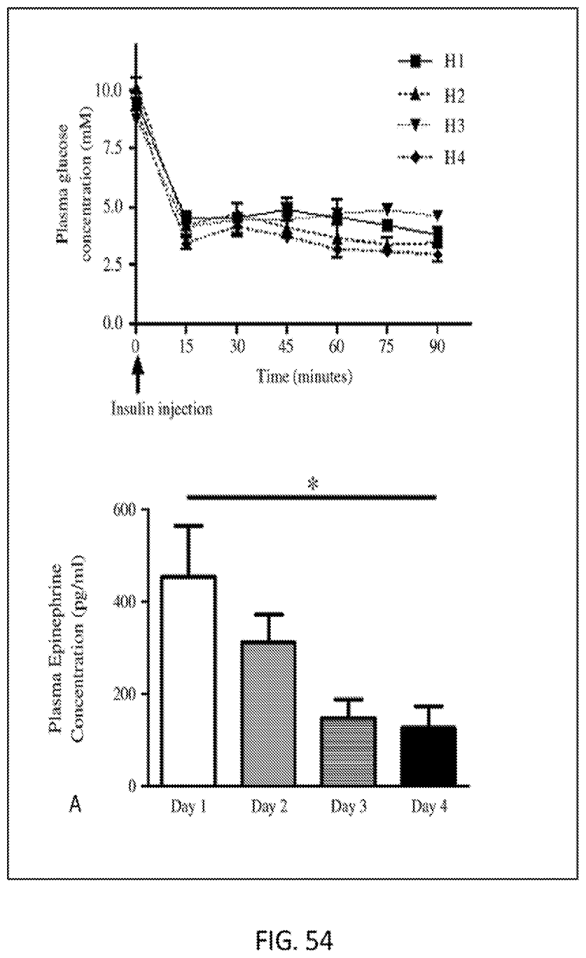

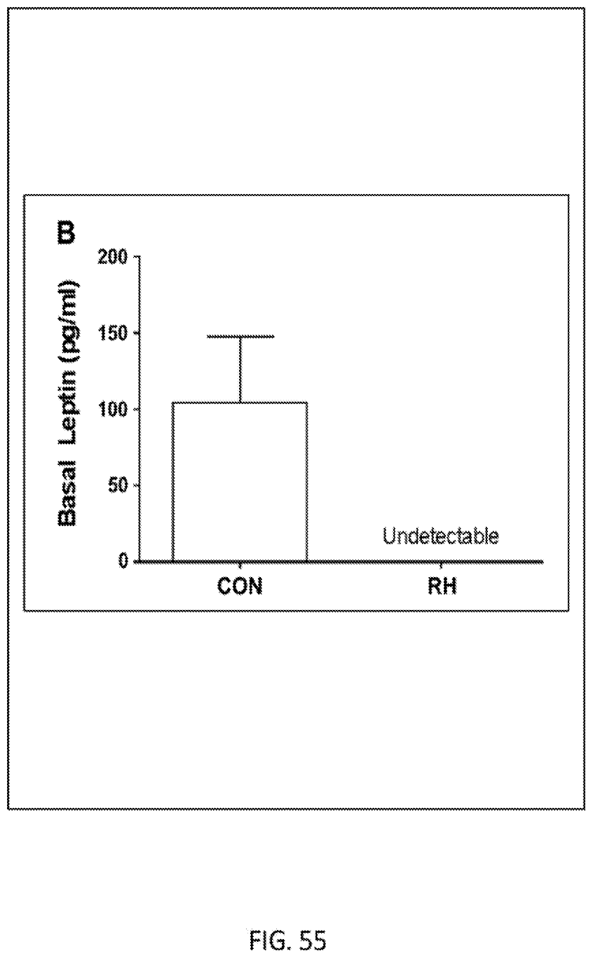

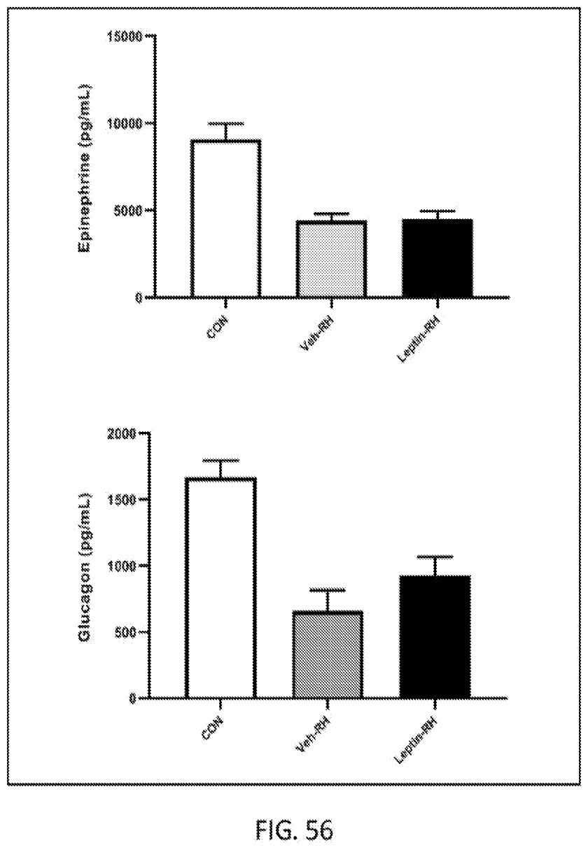

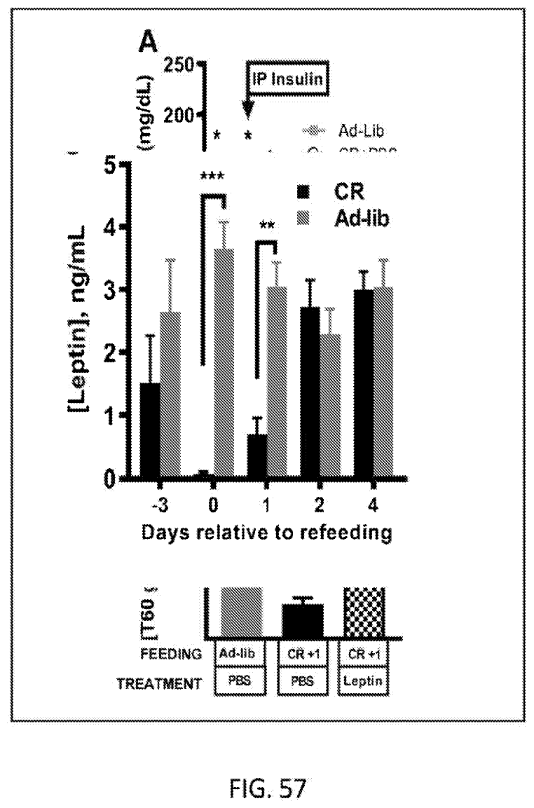

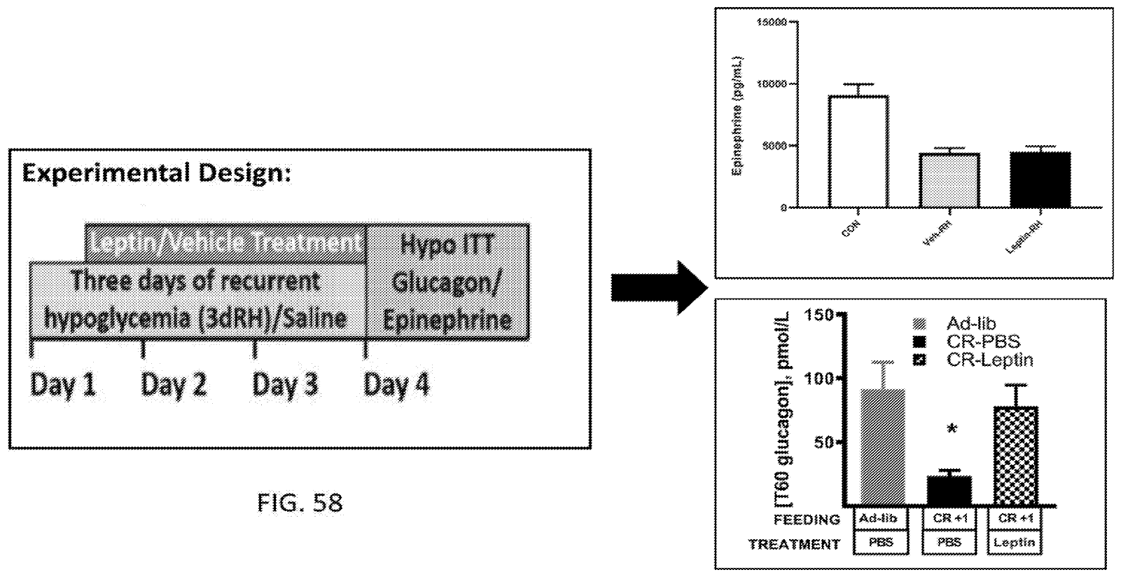

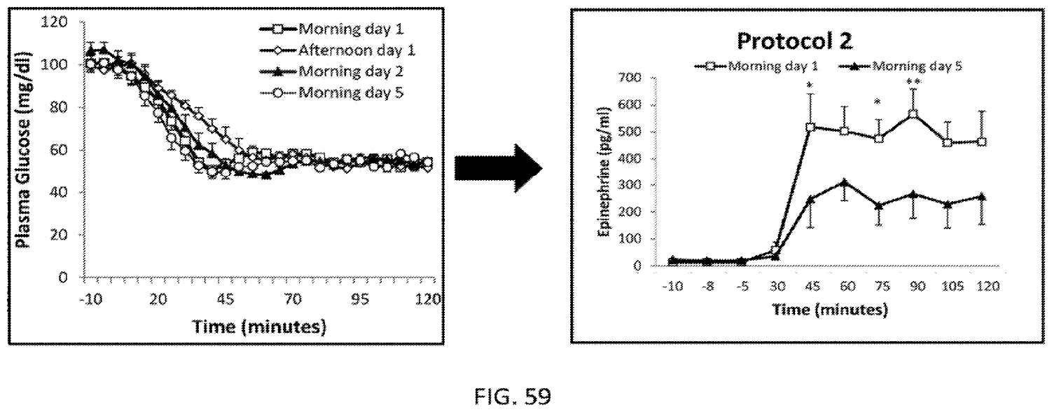

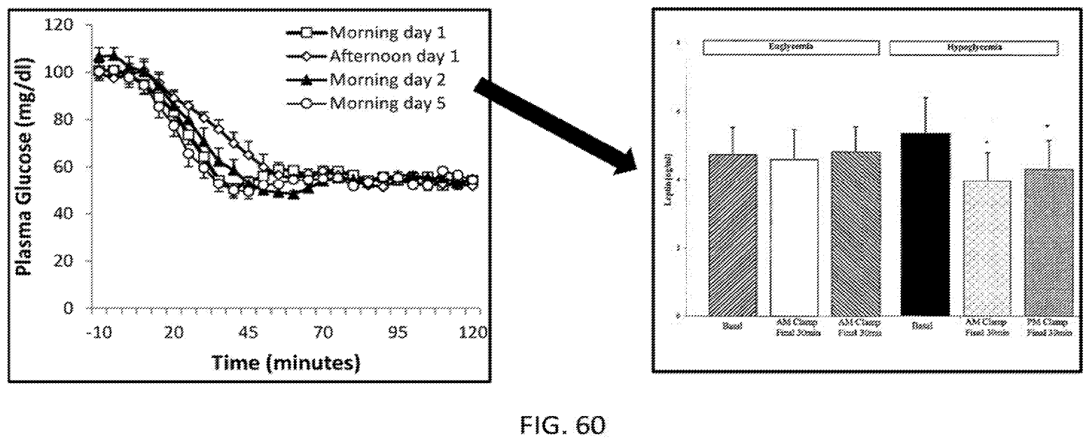

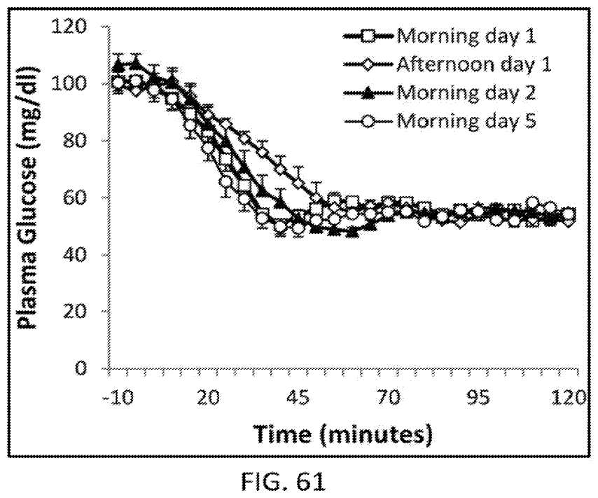

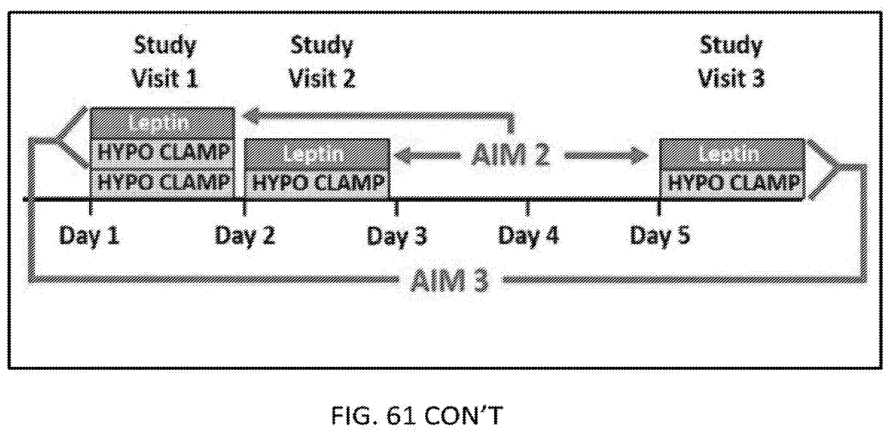

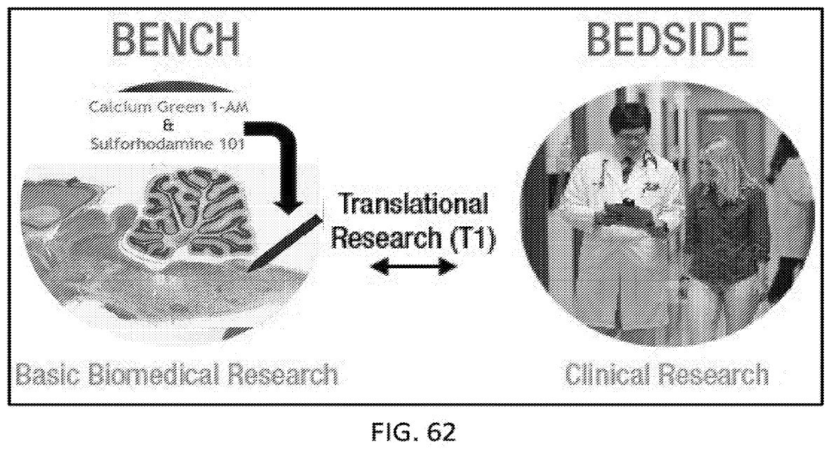

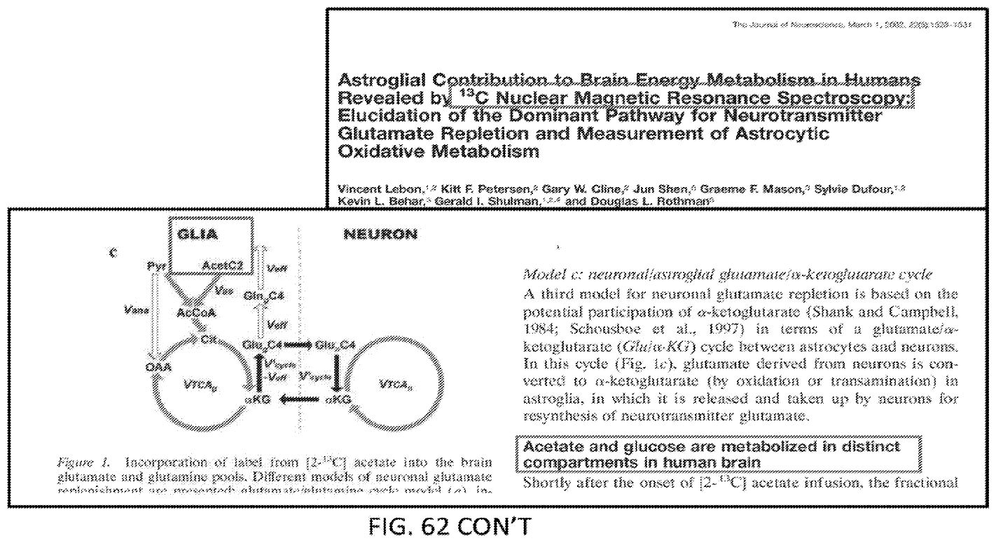

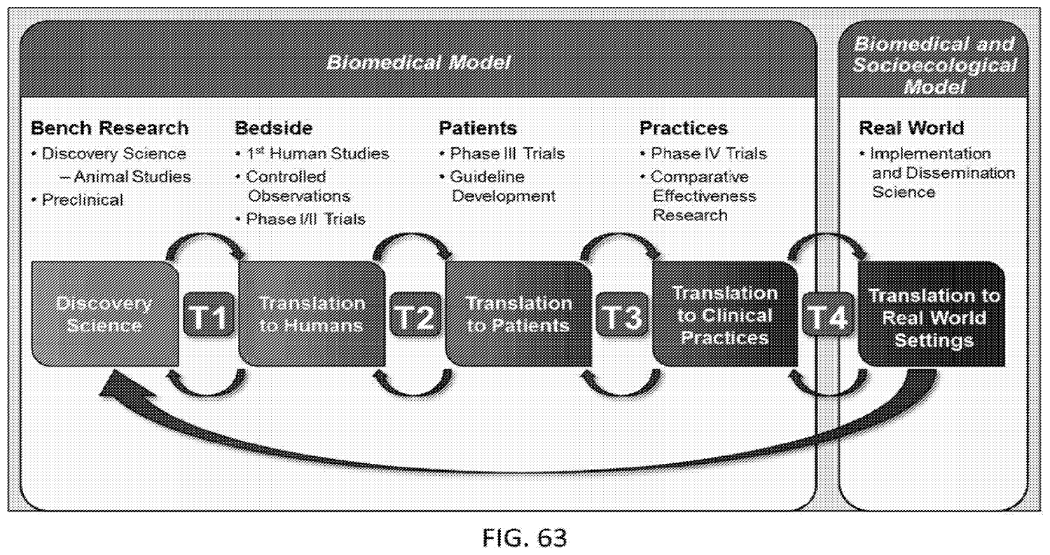

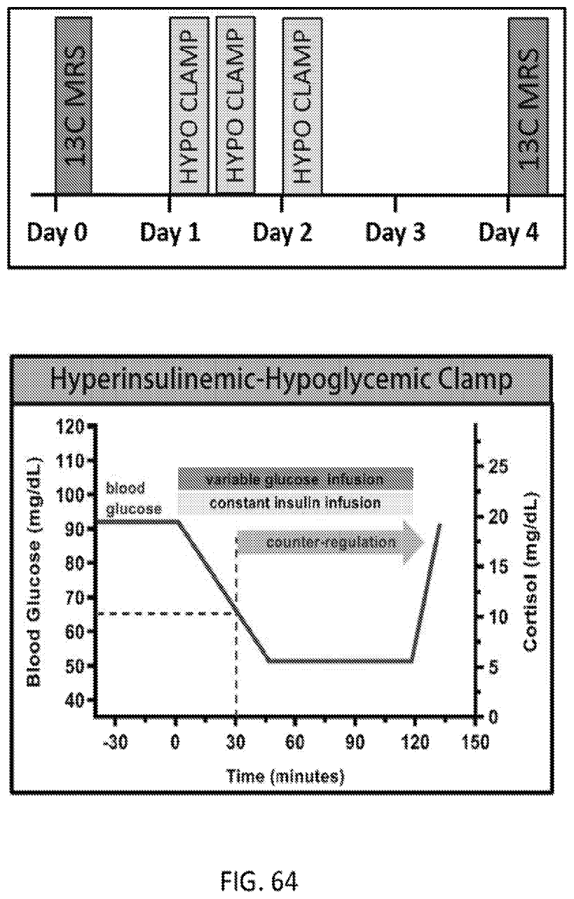

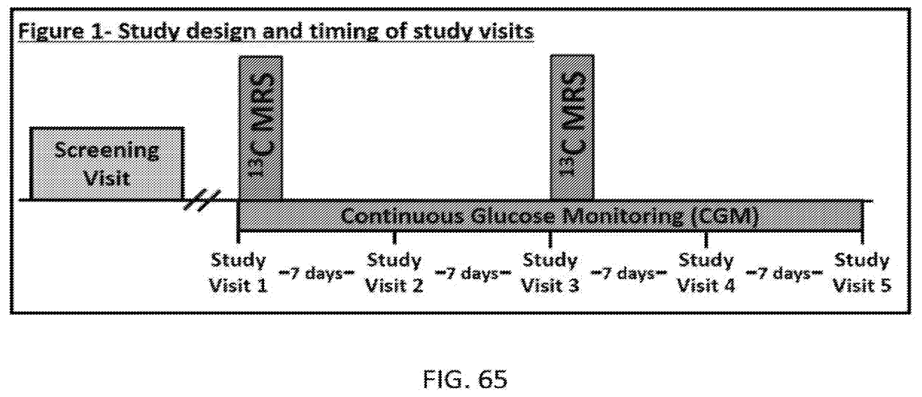

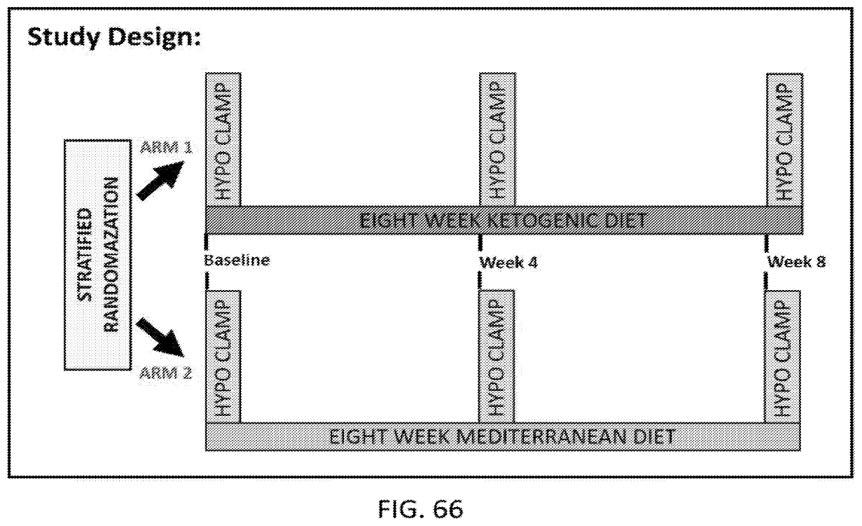

HAAF and Leptin
There is experimental evidence that leptin signaling is associated with HAAF: women with anorexia, and thus who have hypoleptinemia, display HAAF-like symptoms (FIG. 51) (Nakagawa, et al., (1985) Endocrinologia japonica) and (FIG. 52) (Fujii et al. (1989) Acta endocrinologica).
Exposure to a single bout of hypoglycemia in humans leads to reductions in serum leptin levels (FIG. 53) (Sandoval, et al. (2003) Journal of diabetes and Its Complications 17(6): 301-306) and (FIG. 54) (Osundiji, et al., (2011) Metabolism: clinical and experimental 60(4): 550-556.
Exposure to daily bouts of hypoglycemia for three days straight in rats leads to extreme hypoletinemia (FIG. 55) (Reno et al. (2015) American Journal of Physiology, Endocrinology, and Metabolism).
Acute leptin supplementation during final day of hypoglycemia does not rescue the effect (FIG. 56) (Reno et al. (2015) American Journal of Physiology).
Leptin supplementation during caloric restriction in mice resuces the HAAR-like symptoms induced by starvation (FIG. 57).
Experimental Aim 1: Validate whether leptin supplementation during recurrent hypoglycemia in rats reverse HAAF-like symptoms (FIG. 58).
Experimental Aim 2: Validate whether recurrent exposure to hypoglycemia leads to reduced leptin levels in humans (FIGS. 59, 60 and 61).

### Example 9

Not wishing to be bound by theory, the following are examples of research studies:

Clinical Studies:

1. Validate whether leptin treatment prevents the development of HAAF in non-diabetic subjects using the previously validated five-day recurrent hypoglycemia paradigm. There will be two arms: one receiving leptin and the other receiving a placebo. One endpoint will be epinephrine levels during the clamp procedure on day 5.

2. Clinical observational study in type 1 diabetes patients treated with insulin and leptin (Arm 1) or insulin alone (Arm 2). It will contain two phases: Phase A is a 12 week run-in to achieve stable insulin dosing in both arms, and Phase B is a 12 week observational period to determine differences in hypoglycemic events between the two arms. One endpoint will be % times with glucose ≤50 mg/dL measured via continuous glucose monitoring.

Animal Studies:

1. Determine the kinetics of the fall in serum leptin levels in rat exposed to 3 days of recurrent hypoglycemia (animal model of HAAF) and how fast it recovers in the 3 days afterwards.

2. Determine if leptin acts centrally or peripherally to prevent HAAF in rats. Two groups of experimental rats, one gets leptin via intra peritoneal injection and another get leptin directly in the brain, intracerebroventricular injection, during exposure to recurrent hypoglycemia.

3. Determine if other stimuli that produce hypoleptinemia, e.g. chronic cold exposure, leads to the loss of hypoglycemic counterregulation in mice.

4. Determine the effect of 60% caloric restriction in mice on hypoglycemia-induced epinephrine levels using the hyperinsulinemic hypoglycemic clamp technique.

5. Determine the neural circuitry involved in mediating the effect of hypoleptinemia on hypoglycemic counterregulation in mice. This would involve various mechanisms for inactivating specific populations of leptin neurons in the brain, i.e. various strains of transgenic mice and virus injections, and then measuring the downstream effects on counterregulation.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of preventing a hypoglycemia-associated complication, the method comprising administering to a subject in need thereof a therapeutically effective amount of a composition comprising leptin, a fragment thereof, and/or a leptin receptor agonist.

2. A method of treating a hypoglycemia-associated complication, the method comprising administering to a subject in need thereof a therapeutically effective amount of a composition comprising leptin, a fragment thereof, and/or a leptin receptor agonist.

3. The method of claim 1 or 2, wherein the hypoglycemia-associated complication is HAAF.

4. The method of claim 1 or claim 2, wherein the composition is administered prior to the onset of severe hypoglycemia.

5. The method of claim 1 or claim 2, wherein the composition is administered at about the same time as the onset of severe hypoglycemia.

6. The method of claim 1 or claim 2, wherein the leptin comprises human recombinant leptin.

7. The method of claim 1 or claim 2, wherein the method comprises administering a short course of leptin.

8. The method of claim 1 or claim 2, wherein the method comprises chronic administration of leptin.

9. The method of claim 1 or claim 2, wherein the subject in need thereof is afflicted with diabetes.

10. The method of claim 1 or claim 2, wherein the composition further comprises insulin.

11. A therapeutic combination composition comprising leptin, a fragment thereof, or a leptin receptor agonist, and at least one additional active agent.

12. The therapeutic combination composition of claim 11, wherein the composition further comprises a pharmaceutically acceptable carrier, diluent, or excipient.

13. The therapeutic combination composition of claim 11, wherein the at least one additional active agent comprises insulin or an insulin secretagogue.

14. The therapeutic combination composition of claim 11, wherein the at least one additional active agent comprises insulin.

15. The therapeutic combination composition of claim 13, wherein the insulin secretagogue comprises sulfonylurea or glinides.
